(19) United States
(12) Patent Application Publication
(10) Pub. No.: US 2024/0050950 A1
(43) Pub. Date: Feb. 15, 2024

(54) GRADIENT-BASED MICROFLUIDIC CIRCUIT, DEVICE, AND METHOD FOR PERFORMING AN ASSAY

(71) Applicant: CORNELL UNIVERSITY, Ithaca, NY (US)

(72) Inventors: Alireza ABBASPOURRAD, Ithaca, NY (US); Morteza AZIZI, Ithaca, NY (US)

(21) Appl. No.: 18/259,101

(22) PCT Filed: Dec. 22, 2021
(86) PCT No.: PCT/US2021/064928

§ 371 Date: Jun. 23, 2023

Related U.S. Application Data
(60) Provisional application No. 63/130,172, filed on Dec. 23, 2020.

(51) Int. Cl. B01L 3/00

(52) U.S. Cl. CPC B01L 3/502761; B01L 2200/0694; B01L 2300/0883; B01L 2300/0864

(57) ABSTRACT

The present disclosure relates to a microfluidic circuit comprising an inlet port; an outlet port; a main channel fluidically connecting the inlet port and the outlet port; and a series of dead-end microchambers of differing volumes, where each microchamber is individually fluidically connected to the main channel via a side channel. The present disclosure also relates to a microfluidic device comprising a support layer; a substrate layer disposed on the support layer; and one or more microfluidic circuits of the present disclosure, where the one or more circuits are disposed within the substrate layer. Also disclosed is a method for performing an assay.

This application claims the benefit of U.S. Provisional Patent Application Ser. No. 63/130,172, filed Dec. 23, 2020, which is hereby incorporated by reference in its entirety.

### FIELD

This disclosure relates to gradient-based microfluidic circuits, devices, and methods for performing an assay.

### BACKGROUND

To decipher the functions and side-effects between an organic small-molecule (approximately <900 Daltons, but even larger molecules may be used) and a biological species (e.g., live eukaryotic and prokaryotic cells), conventional biological assays often use 96 well-plates, in which each well is used to test a different small-molecule concentration (Alsenaid et al., “Biologics and Small Molecules in Patients with Scalp Psoriasis: A Systematic Review,” J. Dermat. Treatment: 1-10 (2020), Mosquera et al., “Cellular Uptake of Nanoparticles Versus Small Molecules: A Matter of Size,” Accounts of Chemical Research 51:2305-2313 (2018); Sarzi-Puttini et al., “Systemic Rheumatic Diseases: From Biological Agents to Small Molecules,” Autoimmunity reviews 18(6):583-592 (2019)). However, sample preparation is time-consuming, costly, and labor-intensive, often requiring large reagent volumes (Lamb et al., “The Connectivity Map: Using Gene-Expression Signatures to Connect Small Molecules, Genes, and Disease,” Science 313:1929-1935 (2006); Stockwell, “Exploring Biology with Small Organic Molecules,” Nature 432:846-854 (2004)). To address these issues, robotics can shorten assay times (Reddy et al., “Point-of-Care Sensors for the Management of Sepsis,” Nature Biomed Eng. 2:640-648 (2018); Wootton et al., “Analog-to-Digital Drug Screening,” Nature 483:43-44 (2012)). However, such systems are generally bulky and expensive, which can impede their use in biological applications, particularly in resource-limited settings (Huang et al., “Smartphone-Based Analytical Biosensors,” Analyst 143(22):5339-5351 (2018); Liu et al., “Point-of-Care Testing Based on Smartphone: The Current State-of-the-Art (2017-2018),” Biosensors and Bioelectronics 132:17-37 (2019); Xu et al., “Discovery and Functional Characterization of a Yeast Sugar Alcohol Phosphatase,” ACS Chem. Biology 13:3011-3020 (2018)).

As an alternative, miniaturization of biological assays using microfluidics may be an ideal solution for improving throughput and lowering costs (Avesar et al., “Rapid Phenotypic Antimicrobial Susceptibility Testing Using Nanoliter Arrays,” Proc. Natl. Acad. Sci. 114(29):E5787-E5795 (2017); Azizi et al., “Nanoliter-Sized Microchamber/Microarray Microfluidic Platform for Antibiotic Susceptibility Testing,” Analytical Chemistry 90(24):14137-14144 (2018); Baltekin et al., “Antibiotic Susceptibility Testing in Less Than 30 Min Using Direct Single-Cell Imaging,” Proc. Natl. Acad. Sci. 114(34):9170-9175 (2017); Campbell et al., “Microfluidic Advances In Phenotypic Antibiotic Susceptibility Testing,” Biomedical Microdevices 18(6):103 (2016); Hong et al., “Antibiotic Susceptibility Determination Within One Cell Cycle at Single-Bacterium Level by Stimulated Raman Metabolic Imaging,” Analytical Chemistry 90(6):3737-3743 (2018); Kao et al., “Gravity-Driven Microfluidic Assay for Digital Enumeration of Bacteria and for Antibiotic Susceptibility Testing,” Lab on a Chip 20(1):54-63 (2020); Kim et al., “Recent Developments of Chip-Based Phenotypic Antibiotic Susceptibility Testing,” BioChip Journal 13(1):43-52 (2019); Li et al., “Adaptable Microfluidic System for Single-Cell Pathogen Classification and Antimicrobial Susceptibility Testing,” Proc. Natl. Acad. Sci. 116(21):10270-10279 (2019); Mohan et al., “A Multiplexed Microfluidic Platform for Rapid Antibiotic Susceptibility Testing,” Biosensors and Bioelectronics 49:118-125 (2013); Yang et al., “All-Electrical Monitoring of Bacterial Antibiotic Susceptibility in a Microfluidic Device,” Proc. Natl. Acad. Sci. 117(20):10639-10644 (2020)). Small-molecule concentration-based biological assays can be performed in microfluidic systems with significantly improved precision (Leonard et al., “Unraveling Antimicrobial Susceptibility of Bacterial Networks on Micropillar Architectures Using Intrinsic Phase-Shift Spectroscopy,” ACS Nano 11:6167-6177 (2017); Li et al., “Adaptable Microfluidic System for Single-Cell Pathogen Classification and Antimicrobial Susceptibility Testing,” Proc. Natl. Acad. Sci. 116:10270-10279 (2019); Syal et al., “Antimicrobial Susceptibility Test with Plasmonic Imaging and Tracking of Single Bacterial Motions on Nanometer Scale,” ACS Nano 10:845-852 (2016)). For example, one of the most studied platforms for this application uses a well-known “Christmas tree” technique (Jang et al., “An Integrated Microfluidic Device for Two-Dimensional Combinatorial Dilution,” Lab on a Chip 11(19):3277-3286 (2011); Kim et al., “A Programmable Microfluidic Cell Array for Combinatorial Drug Screening,” Lab on a Chip 12(10):1813-1822 (2012); Lim et al., “A Microfluidic Spheroid Culture Device with a Concentration Gradient Generator for High-Throughput Screening of Drug Efficacy,” Molecules 23(12):3355 (2018)). This technique produces precise concentrations of a drug in a microfluidic chip, but it needs precision instruments (such as syringe pumps) to robustly control the flowrates of two loading fluids. Moreover, such designs require time-consuming sample-loading protocols that cannot be easily automated, and more importantly, lack enough throughput to enable simultaneous testing of negative controls and a wide concentration range of positive samples in a single test.

The present disclosure is directed to overcoming these and other deficiencies in the art.

### SUMMARY

One aspect of the present disclosure relates to a microfluidic circuit comprising an inlet port; an outlet port; a main channel fluidically connecting the inlet port and the outlet port; and a series of dead-end microchambers of differing volumes, where each microchamber is individually fluidically connected to the main channel via a side channel.

Another aspect of the present disclosure relates to a microfluidic device comprising a support layer; a substrate layer disposed on the support layer; and one or more microfluidic circuits of the present disclosure, where the one or more circuits are disposed within the substrate layer.

A further aspect of the present disclosure relates to a method for performing an assay. This method involves loading a first reagent solution into the inlet port of a microfluidic device of the present disclosure. A second reagent solution is loaded into the inlet port, and then an isolating solution is loaded into the inlet port. The method further involves detecting an interaction between the first reagent solution and the second reagent solution in one or more of the microchambers.

The present disclosure relates to a novel multi-volume microchamber-based microfluidic (“MVM²”) platform that is designed to produce a spontaneous and broad gradient of small-molecule concentrations within a single test. Antibiotic susceptibility testing and sugar phosphate toxicity (for bacteria and yeast cells, respectively) (Gibney et al., “Common and Divergent Features of Galactose-1-Phosphate and Fructose-1-Phosphate Toxicity In Yeast,” Molecular Biology Cell 29(8):897-910 (2018); Johnston et al., “Nitrate and Phosphate Transporters Rescue Fluoride Toxicity in Yeast,” Chemical Research Toxicology 32(11):2305-2319 (2019); Machado et al., “The Galactose-Induced Decrease in Phosphate Levels Leads to Toxicity in Yeast Models of Galactosemia,” Biochimica et Biophysica Acta (BBA)-Molecular Basis of Disease 1863(6):1403-1409 (2017); Xu et al., “Discovery and Functional Characterization of a Yeast Sugar Alcohol Phosphatase,” ACS chemical biology 13:3011-3020 (2018); which are hereby incorporated by reference in their entirety) were studied as two clinical models to demonstrate the versatility of the MVM² platform. The loading time, which is the only parameter needed to be controlled by an operator for running the MVM² platform, was obtained for a wide-range of commercial biological small-molecules in the market including anticancer drugs, antibiotics, and antifungals. Overall, with the MVM² design, it is possible to rapidly determine precise effects of small-molecules in a broad concentration range with high throughput and low cost, and in a manner that is readily adaptable for automation.

Compared with previous milestones in developing microfluidic platforms for biological assays, the microfluidic circuit and microfluidic device of the present disclosure is, to the best of Applicant's knowledge, the first microfluidics device that is able to: (i) test small-molecules on both eukaryotic and prokaryotic cells; (ii) work in a high-throughput mode with an extended range of small-molecule concentrations (e.g., three orders of magnitude), while also including negative controls; (iii) exploit a low-cost microfluidics chip (˜$1 each) using a facile operation protocol; and (iv) prepare the desired sample concentrations precisely using fluid dynamics with minimal human intervention. Moreover, future integration of this platform with other technologies, such as complementary metal oxide semiconductor imaging, or electrochemical responses, could employ this platform for numerous biological assays, such as cancer cell biology, cell signaling, protein/small-molecule interactions, pesticide analysis, etc. Building from this MVM² concept, further advanced platforms made possible by the easily modified MVM² features are envisioned, which could be applied to even broader future biological and non-biological analytical applications.

### BRIEF DESCRIPTION OF THE DRAWINGS

FIG. 1 is a top view of one embodiment of a microfluidic circuit of the present disclosure comprising an inlet port; an outlet port; a main channel fluidically connecting the inlet port and the outlet port; and a series of dead-end microchambers of differing volumes (forming a gradient from smallest to largest going in the direction of the inlet to the outlet. Each of the microchambers is individually fluidically connected to the main channel via a side channel.

FIG. 2 is a perspective, partial cut-away view of one embodiment of a microfluidic device of the present disclosure comprising a microfluidic circuit of the present disclosure disposed within the substrate layer of the microfluidic device.

FIG. 3 is an amplified view of section 3 of the microfluidic circuit shown in the microfluidic device of FIG. 2.

FIG. 4 is a perspective view of one embodiment of a microfluidic device of the present disclosure with the top portion of the substrate completely cut away to show the underlying microfluidic circuits formed in the substrate. The microfluidic device shown comprises four microfluidic circuits connected via connecting channel.

FIG. 5 is a top view of one embodiment of a microfluidic device of the present disclosure with the top portion of the substrate completely cut away to show the underlying microfluidic circuits formed in the substrate. In the particularly embodiment shown, illustrated is the design of the four microfluidic circuits for performing an assay with high, medium, low, and negative (control) concentrations of a test reagent.

FIGS. 6A-F are illustrations of one embodiment of MVM² microfluidic device features and sample loading principles, demonstrating the design of the negative, low, medium, and high concentration small-molecule main-channels. FIG. 6A is a schematic structure of the MVM² platform in which only microchambers R₁-R₅ are shown for simplicity. Inset: Schematic illustration of a microchamber connected to the main channel. FIG. 6B shows the features of the MVM² platform, demonstrating the design of the negative, low, medium, and high concentration small-molecule main-channels. The assay loading steps, include: FIG. 6C: biological species loading (uniform concentration throughout); FIG. 6D: small-molecule loading into the positive main-channels by diffusion at three orders of magnitude (C₀, 0.1 C₀, and 0.01 C₀); FIG. 6E: stopping the loading of the small-molecules by washing the main-channels with a biocompatible oil to isolate the microchambers containing the biological species and small-molecules; and FIG. 6F: blocking the inlets and outlets with sterilized medical tape and letting the loaded small-molecules uniformly distribute in the microchambers. As a result, the smallest and largest microchambers in each row feature the highest and lowest concentrations of the small-molecule in the low, medium, and high ranges. Moreover, the smallest microchambers of the low and medium ranges feature identical concentrations as the largest microchambers of the medium and high ranges, respectively, ensuring there is continuity within the concentrations tested.

FIGS. 7A-F show characterization of resazurin loading in the MVM² platform. FIG. 7A is an illustration with a dashed schematic arrow (length Lo) for monitoring the resazurin diffusion into the microchamber, with three points designated (m, n, and p), representing the starting point of the side-channel inlet, the junction where the inlet connects to the microchamber, and the farthest point in the microchamber from the side channel inlet, respectively. FIGS. 7B and 7C are graphs showing resazurin concentration profiles determined by CFD simulations for microchambers R₁ and R₁₂, respectively. Microchambers R₁ and R₁₂ were chosen to study the kinetics of resazurin diffusion as the diffusion trend can be generalized for the 10 remaining microchambers, R₂-R₁₁, which are of intervening size. FIG. 7D is a graph showing before (i) and after (ii) the uniform distribution of resazurin into the side-channel and the corresponding microchamber R₁. FIG. 7E is a photograph showing the experimentally OBSERVED GRADIENT CONCENTRATION PROFILE (GCP) OF RESAZURIN LOADING IN MICROCHAMBERS R₁-R₁₂. FIG. 7F is a graph comparing the normalized GCPs obtained by experimental (n=5) and CFD simulation of resazurin with the theoretical data (obtained from the microchambers' volumes and normalization of the small-molecule concentrations). Scale-bar: 200 μm. The shaded grey area corresponds to the error bars.

FIGS. 8A-E show the correlation between the small-molecule molar volume and loading time in the MVM² platform. FIG. 8A is an illustration of CFD simulations for microchambers R₁ and Ru confirm the relationship between the diffusion coefficients and loading times of any two small-molecules, satisfying the equation D₁×t₁=D₂×t₂. The CFD simulations show identical concentration profiles (i.e., same color patterns) for two small-molecules with different loading times, satisfying the relationship D₁×t₁=D₂×t₂. FIG. 8B is a photograph showing time-lapse calcein diffusion into microchamber R₁. The fluorescent calcein gradually diffuses into the side-channel and the connected microchamber over the loading time. Scale-bar: 200 μm. FIG. 8C is a graph showing validation of the relationship Molar volume₁×t₂=Molar volume₂×t₁ between the loading times and their molar volumes, tested for three fluorescent dyes—resazurin, fluorescein, and calcein. The obtained trend-line fit of the loading times for resazurin, fluorescein, and calcein is described by Loading time=0.4954×molar volume−3.7623, with R²=0.9885. This equation can be used to obtain the loading times for other small-molecules applicable in the MVM² platform (** and ***: p values<0.01 and 0.001, respectively). FIG. 8D is an illustration showing a technique for testing the correlation between the small-molecule diffusion coefficients and molar volumes. The small-molecule solution is gently loaded at the bottom of a cuvette using a chromatography syringe. FIG. 8E is a graph showing normalized concentration versus diffusion time for four tested small molecules, including ampicillin, cefuroxime, resazurin, and nalidixic acid.

FIGS. 9A-F relate to examples showing the functionality of the MVM² platform. The antimicrobial susceptibility assay for E. coli 541-15 as a bacteria species susceptible to gentamicin after incubation for 4 h is shown in FIGS. 9A-D. FIG. 9A is a photograph showing red fluorescent mode (representing resazurin reduction correlated with bacterial metabolites) and FIG. 9B is a photograph showing green fluorescent modes (visualizing the GFP-labeled bacterial growth/inhibition). Kanamycin was used at an appropriate concentration (50 μg/mL) in the bacteria/resazurin suspension, avoiding bacteria-inserted GFP plasmid repulsion during the antimicrobial resistant assay. FIG. 9C is a graph of gray values for the positive microchambers in the medium range (1-10 μg/mL) over 4-h antimicrobial susceptibility testing. The gray values were obtained by converting the fluorescent intensity produced from resazurin-reduction in the culture medium, which correlates with the E. coli 541-15 bacterial growth. FIG. 9D is a graph showing determination of the MIC of the E. coli 541-15/gentamicin pair. FIG. 9E is a graph showing the validation of the MVM² platform functionality using the gold standard broth microdilution technique. FIG. 9F is an illustration of Crohn's diseases' clinically isolated E. coli LF82, E. faecalis 44, and K. pneumoniae 578 tested via four clinically relevant antibiotics including: ampicillin, nalidixic acid, lincomycin, and gentamicin. Scale-bar in FIGS. 9A and 9B: 200 μm.

FIGS. 10A-D relate to MVM² device size and features. FIG. 10A is a photograph of one embodiment of an MVM² device showing a bare glass slide on the bottom and a cured PDMS substrate that has been peeled off from a mold and fixed onto the glass slide such that the open surface of the PDMS is facing the glass slide to create channels and chambers with both a bottom (glass slide) and a top (PDMS substrate). Holes have been punched into the top surface of the PDMS substrate at the inlet and outlet ports to provide access to fluids through the PDMS to the microfluidic circuits. FIG. 10B is a scanning electron micrographic (“SEM”) image of the MVM² platform, demonstrating the configuration of the main channel, connecting side channels, and dead-end microchambers. FIG. 10C is an SEM image of microchamber R₁. FIG. 10D is an illustration showing the dimensions of the MVM² device, which has 12 multi-volume microchambers (R₁-R₁₂) along each side of the main channels that range in diameter from 250 to 1130 μm. In this exemplary embodiment, the depth of all the microfluidic circuit features (channels, microchambers, etc.) is equal to 70 μm. The features in the scheme are not to scale.

FIGS. 11A-D relate to the protocol for biological species loading. FIG. 11A is an illustration of the loading of the biological suspension, which occurs at opening A₁, discharging from openings A₂-A₄, while openings B₁-B₄ are kept closed. FIG. 11B is an illustration showing openings A₂-A₄ and B₁-B₄ are then blocked while continuing to load the biological suspension from opening A₁. FIG. 11C is an illustration showing that the biological suspension completely fills each microchamber due to the pressure of the solution flow, which forces entrapped air to escape from the PDMS walls, resulting in a uniformly loading the biological suspension throughout the device. FIG. 11D is a photograph of a time-lapse experimental sample loading using a resazurin red fluorescence dye solution to illustrate the sample loading process into empty microchamber R₁. The scale bars are 200 μm.

FIGS. 12A-D are illustrations of small-molecule loading in the MVM² platform. FIG. 12A: Washing the main negative control channel using the biocompatible oil to isolate the negative microchambers (i.e., no small-molecule loading). Loading low-range (light green color) (FIG. 12B), medium-range (green color) (FIG. 12C), and high-range (dark green color) (FIG. 12D) small-molecule solution into the low-, medium-, and high-range main channels, respectively, followed by washing the channels out using oil to isolate the microchambers.

FIGS. 13A-B are illustrations of a design for MVM² platform while drug is loaded I (FIG. 13A), and two designs for MVM² platform (FIG. 13B). In scenario #1 of FIG. 13B, the side-channel was designed straight in the MVM² platform. However, in scenario #2 of FIG. 13B, the side-channel was designed in serpentine shape in the MVM² platform. The serpentine-shape side-channel leads to designing a more compact MVM² platform.

FIGS. 14A-B Are illustrations of two potential outcomes expected to obtain from MVM² platform. (FIG. 14A) MVM² platform at time zero, t₀. (FIG. 14B) two potential outcomes for (i) a resistant or (ii) susceptible biological species to the tested small-molecule at final time, t_{f}.

FIGS. 15A-C show diffusion in the MVM² platform. FIG. 15A is a time-lapse photograph of resazurin diffusion in microchamber R₁ preloaded with non-selective Mueller-Hinton (MH) culture medium. The fluorescent resazurin gradually diffuses into the side-channel and the connected microchamber over time. Scale-bar: 200 μm. FIG. 15B is a graph of the gray-value versus the normalized side-channel length during the loading time. For this graph, the resazurin fluorescent intensity obtained from FIG. 15A was converted to gray-value to study the kinetics of resazurin loading into microchamber R₁. FIG. 15C is an illustration of CFD simulations of resazurin loading into R₁ and R₁₂, which are the smallest and largest microchambers, respectively.

FIG. 16 is an illustration of CFD and experimental demonstrations of dead-zone formation in side-channel and microchambers. FIG. 16 shows CFD simulation of the MVM² platform to illustrate the potential fluid dead-zone formation.

FIGS. 17A-B are photographs of small-molecule gradient-based concentration failure in MVM² platform. FIG. 17A shows microchamber R₁ in two time-points 240 s (unsaturated one) and 720 s (saturated one). FIG. 17B shows microchamber R₅ was not saturated with resazurin at t₂ while it was at t=1200. At t=1200, microchambers R₁-R₅, are saturated as it continues till microchamber R₁₂ and fails the concept of small-molecule gradient-based concentration formation.

FIG. 18 is a graph showing the kinetics for resazurin full distribution in microchamber Ru.

FIGS. 19A-D relate to GCPs for small-molecules with different diffusion coefficients. FIG. 19A is an illustration showing qualitative concentration profile obtained using CFD simulations for six theoretical small-molecules with different diffusion coefficients (representative of most small-molecules) but the same loading time (1000 s) in the MVM² platform, resulting in different GCPs. FIGS. 19B-C are graphs showing quantitative concentration profiles for six representative small-molecules into microchamber R₁ at 1000 s of loading (FIG. 19B) over the arrow length, and after (FIG. 19C) homogenously distributing into the corresponding microchambers (˜3 min). As shown in FIG. 19B, a small-molecule with a diffusion coefficient of

$$5 \times 1 {0}^{- 9} \frac{{m}^{2}}{s}$$

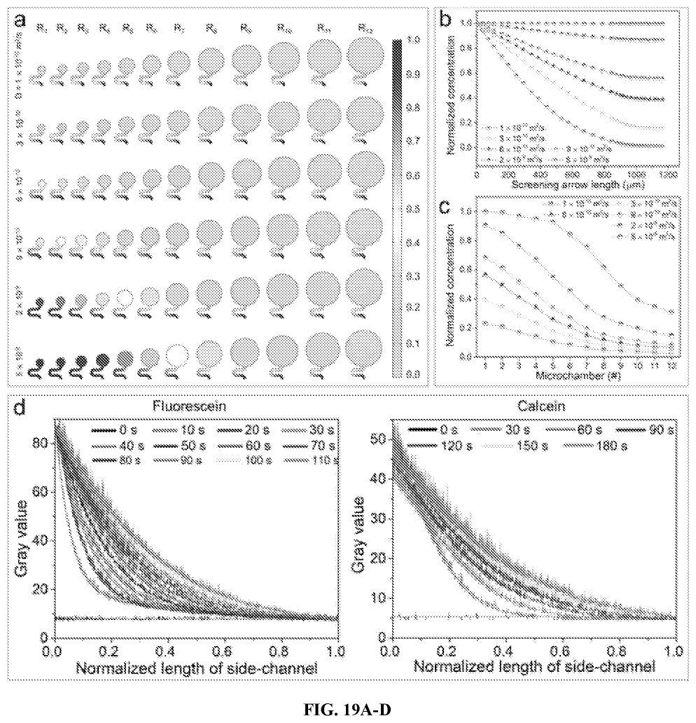

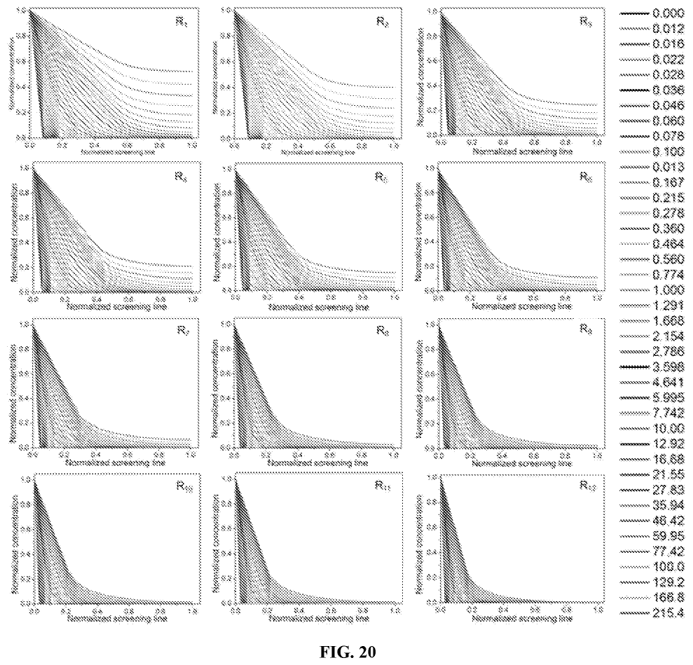

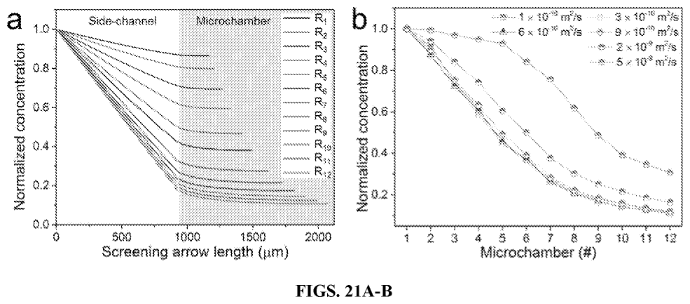

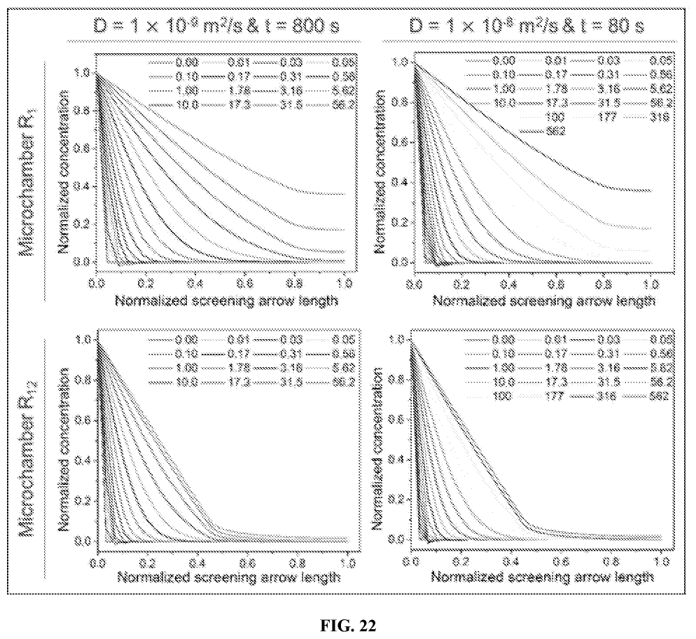

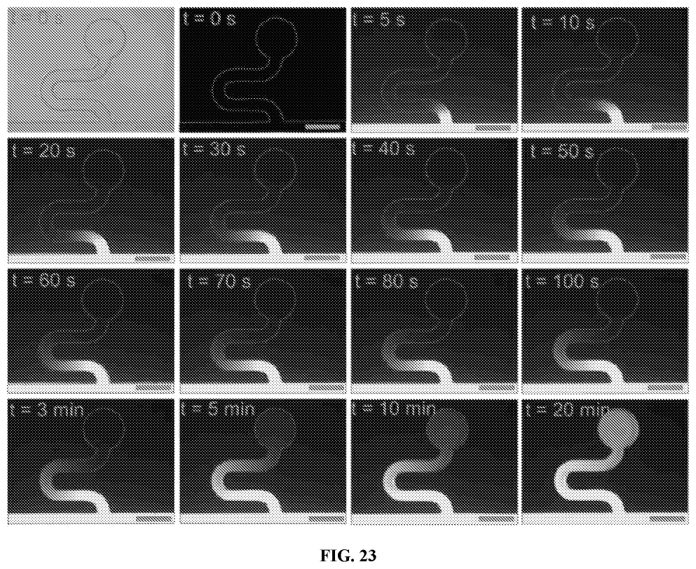

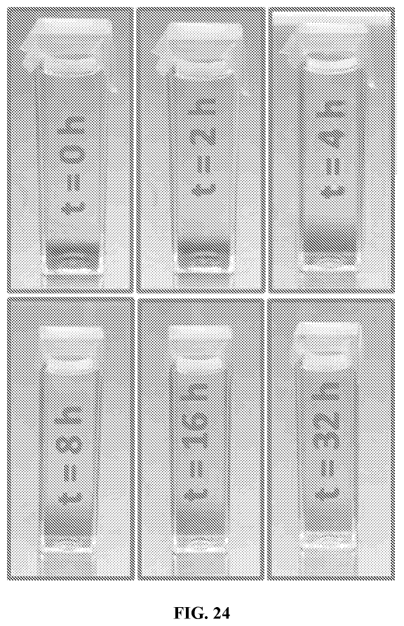

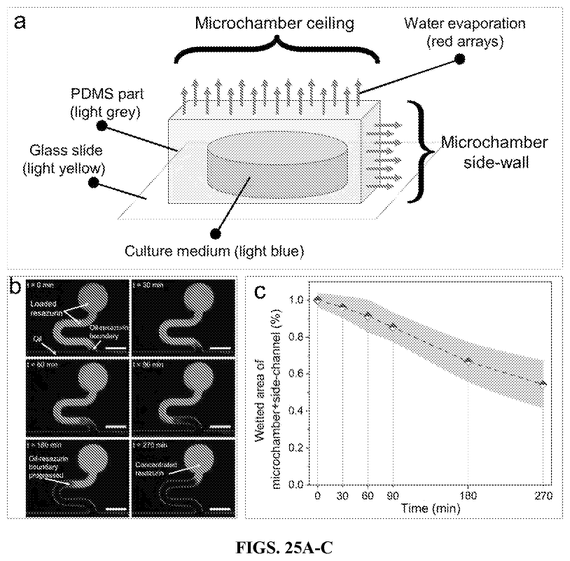

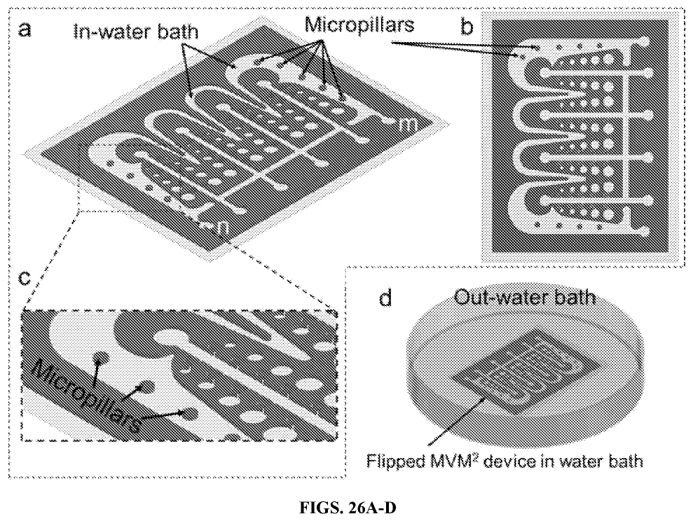

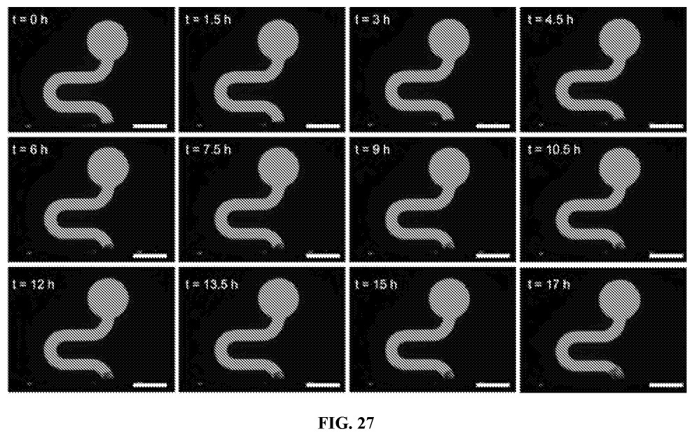

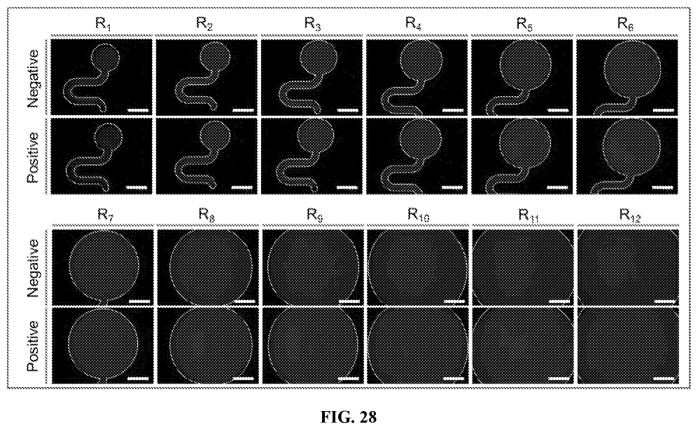

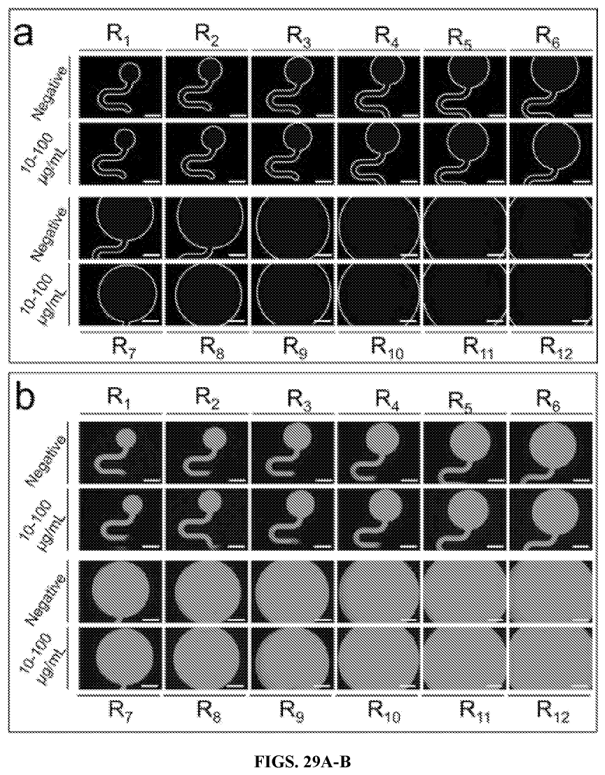

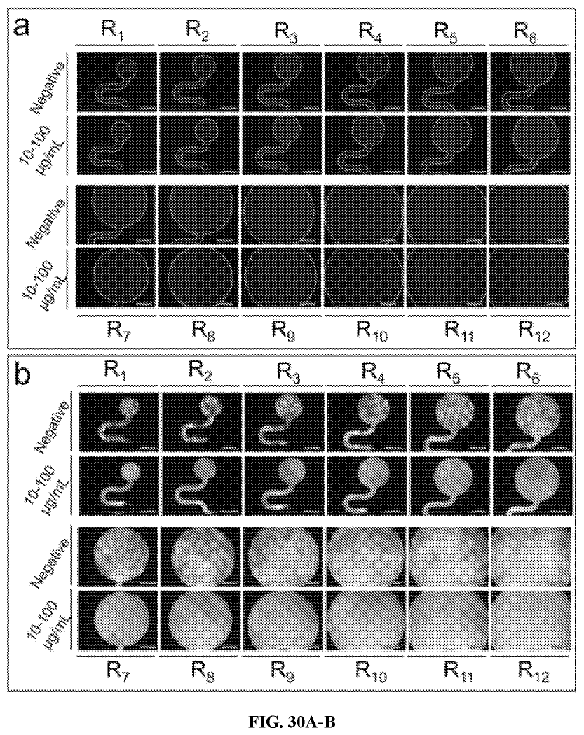

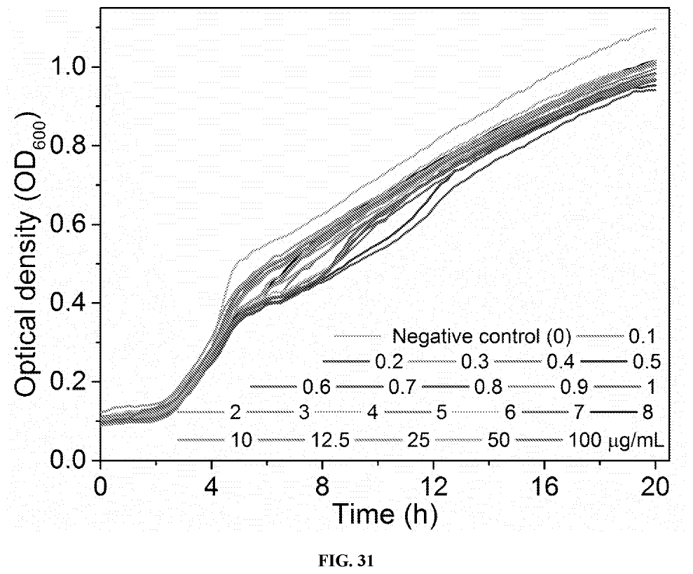

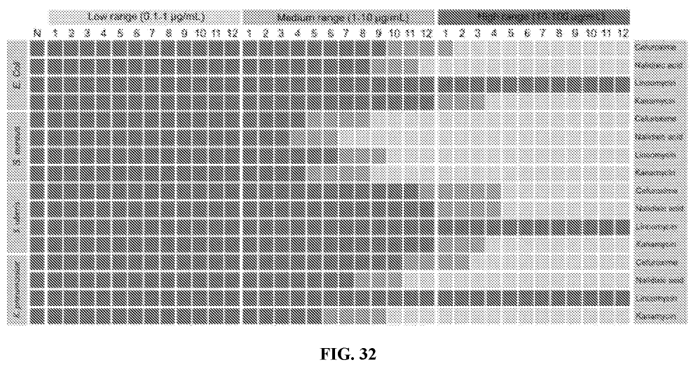

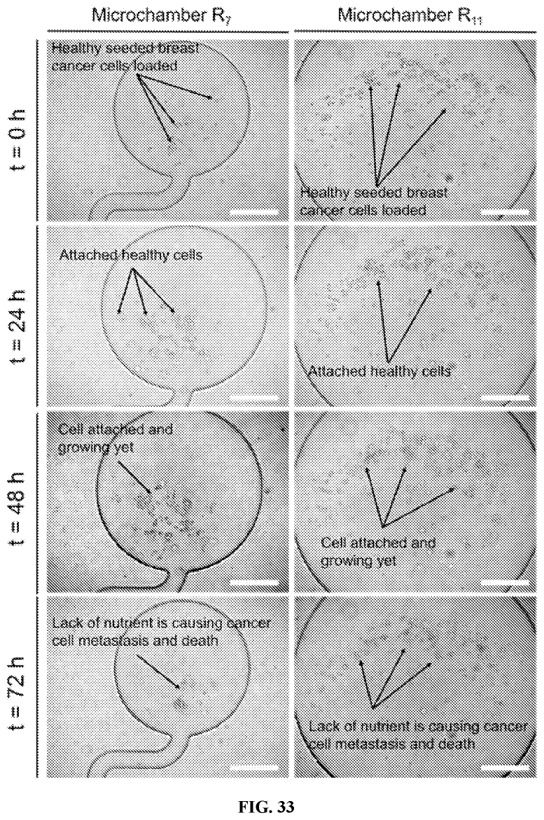

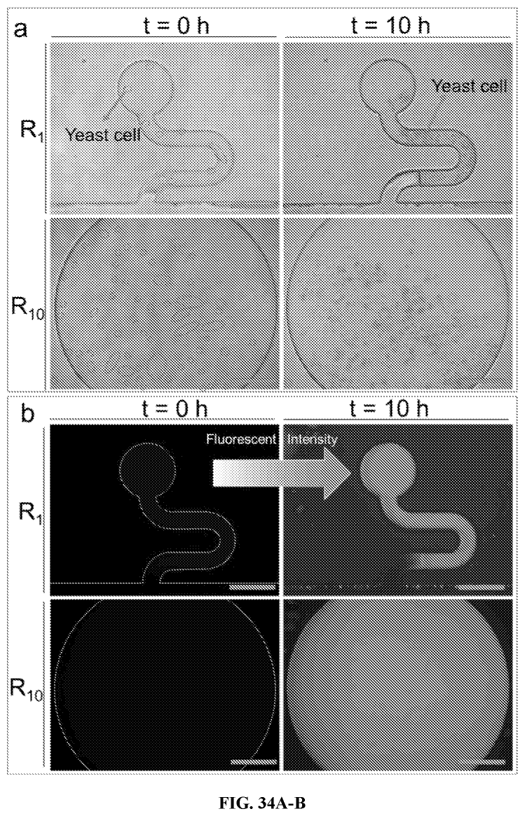

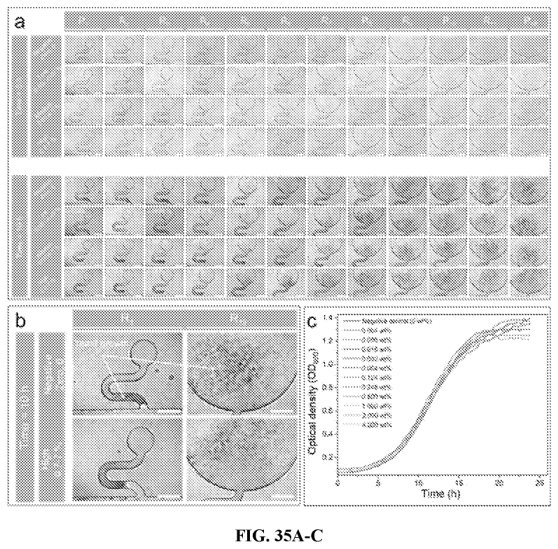

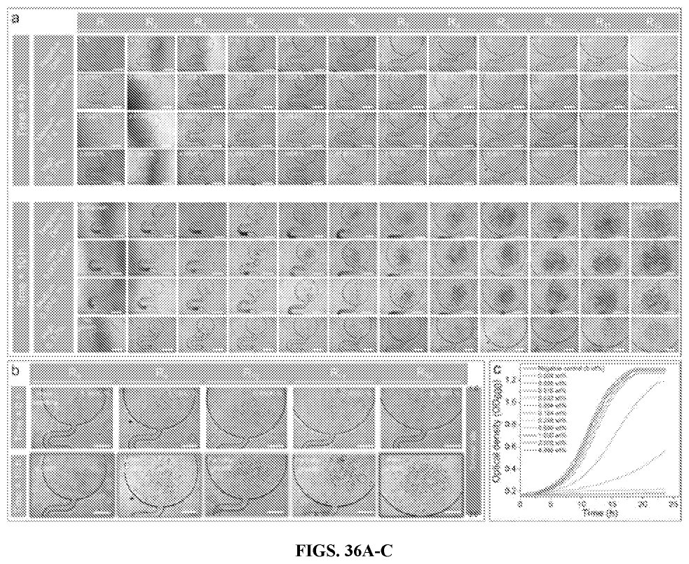

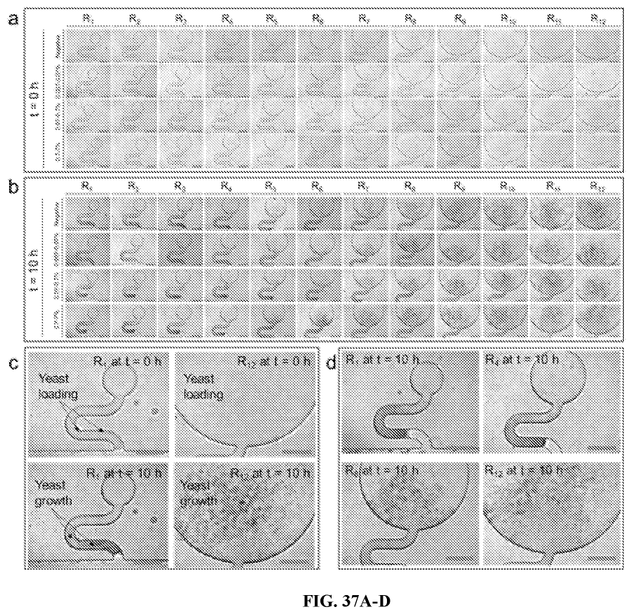

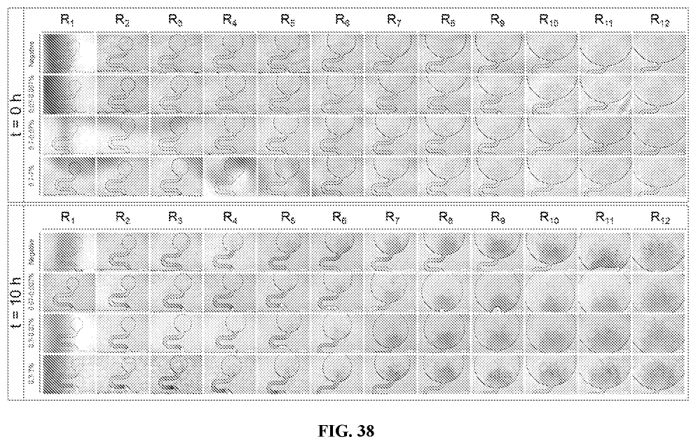

fully saturates the side-channel and microchamber R₁, while others with lower diffusion coefficients are unable to do so (over an unlimited loading time, i.e., ∞, the small-molecule concentration in all microchambers will equal one regardless of the diffusion coefficient). In FIG. 19C, it is shown that the normalized concentration of microchamber R₁-R₁₂ after uniform distribution as it shows, for example, the small-molecule with diffusion coefficient

$$5 \times 1 {0}^{- 9} \frac{{m}^{2}}{s}$$

can almost saturate the microchamber R₁-R₅. FIG. 19D is a graph of g./ray-values converted from the fluorescent intensities to represent the experimental concentration profiles for two fluorescent dyes, fluorescein and calcein, over the normalized length of the side-channel.

FIG. 20 shows graphs of the concentration profiles in microchambers R₁-R₁₂ for a typical small-molecule with diffusion coefficient 5×10⁻⁹ m²/s over a loading time, 215 s. Theoretical obtained normalized concentrations of microchambers R₁-R₁₂.

FIG. 21A shows the concentration profile before uniform distribution of small-molecule into twelve microchambers, microchamber R₁-R₁₂, for a small-molecule with 5×10⁻⁹ m²/s diffusion coefficient. FIG. 21B shows the uniform distribution of small-molecule into twelve microchambers for six different diffusion coefficient and how the lack of control on small-molecule loading through longer loading time, causes failure of one order of magnitude small-molecule GCP into microchambers.

FIG. 22 is a graph showing quantitative concentration profiles for microchambers R₁ and R₁₂ when the relationship D₁×t₁=D₂×t₂ between small-molecule diffusion coefficients and their molar volume is followed.

FIG. 23 is a photograph showing time-lapse fluorescein diffusion into microchamber R₁. The fluorescein dye diffusion was studied over 20 minutes. The scale bars are 200 μm.

FIG. 24 is a photograph showing the visual diffusion assessment of a food grade dye into water using cuvette-spectrophotometry technique during a 32-h course of visual screening.

FIGS. 25A-C relate to water evaporation from the MVM² device. FIG. 25A is an illustration representing the concept behind water evaporation from a microchamber without considering a water bath. FIG. 25B is a photograph showing water evaporation from resazurin-enriched culture medium loaded into isolated microchamber R₁, leading to resazurin fluorescent intensity increment through concentrating small-molecules and variable small-molecule concentration during a typical experiment. FIG. 25C is a graph showing the water evaporation rate from resazurin-enriched culture medium loaded into isolated microchamber R₁.

FIGS. 26A-D are illustrations of techniques implemented on certain embodiments of the MVM² device for avoiding water evaporation from culture medium using a water bath. FIGS. 26A-C illustrate the technique “in-water bath,” and FIG. 26D illustrates the technique “out-water bath,” respectively, making water evaporation equilibrium between two water reservoirs. During an assay, the fluids loaded into the MVM² device (such as the culture medium) can evaporate and escape from the porous PDMS substrate structure (including the side-walls and the roof). By forming a cavity supported by micropillars to prevent the collapse of the cavity that is loaded with a fluid such as water, a water equilibrium balance between the inside and outside of the microchambers can be established through the side-walls. To make another water evaporation equilibrium balance to prevent evaporation between the inside and outside of the microchambers (such as through the top layer or roof), the MVM² device was flipped and placed into a water bath.

FIG. 27 shows photographic images of samples in an experiment performed for 17 h at 37° C. (physiological temperature). Water evaporation from culture medium loaded into MVM² device after modification of the device was avoided. The experiment was performed for 17 h at 37° C. (physiological temperature).

FIG. 28 shows photographic images showing an antimicrobial resistant assay for E. coli 541-15 as a susceptible bacteria to gentamicin at t=0 h. The negative and medium concentration microchamber arrays are shown. Scale-bar: 200 μm.

FIGS. 29A-B are photographs showing an antimicrobial resistant assay for E. coli 541-15 as a resistant bacteria to ampicillin. The red fluorescent intensity emitted from resazurin reduction correlated with E. coli 541-15 metabolites for the negative and high range (10-100 μg/mL) in microchambers R₁-R₁₂ at (a) t=0 h and (b) t=4 h. Scale-bar: 200 μm.

FIGS. 30A-B are photographs showing an antimicrobial resistant assay for E. coli 541-15 as a resistant bacterial species to ampicillin. The green fluorescent intensity visualizes the GFP-labeled E. coli 541-15 bacteria and its growth over 4-h incubation for the negative and high range (10-100 μg/mL) in microchambers R₁-R₁₂ at (a) t=0 h and (b) t=4 h. Scale-bar:

FIG. 31 is a graph showing broth microdilution for a resistant bacterial species. Growth curve (optical density (OD₆₀₀) vs. time) for E. coli 541-15 as a resistant bacterial species exposed to ampicillin at a wide range of ampicillin concentrations plus the negative control (not exposed), obtained using the gold standard broth microdilution technique.

FIG. 32 is an illustration of an antimicrobial R/S profile of clinically isolated bacteria performed using MVM² platform. Bovine mastitis clinically isolated E. coli, S. aureus, S. uberis, and K. pneumoniae tested via four clinically relevant antibiotics including: cefuroxime, nalidixic acid, lincomycin, and kanamycin.

FIG. 33 shows photographic images showing breast cancer cell growth monitoring. Breast cancer cell line was loaded into microchambers (microchamber R₇ and R₁₁ chosen). The growth was monitored within 72 h cell study.

FIGS. 34A-B are photographs showing the capabilities of yeast growth in MVM² device for a long-term run. FIG. 34A shows yeast growth in MVM² platform showing in microchambers R₁ and R₁₀. FIG. 34B shows changing the fluorescence intensity of resazurin as fluorescence chemical indicator in microchambers R₁ and R₁₀ over a 10 h culture study. Scale bar is 200 μm.

FIGS. 35A-C relate to testing glucose against a mutant yrKHK S. cerevisiae yeast strain. FIG. 35A is a photograph showing the yrKHK strain exposed with glucose in 0.007-7 wt % range plus negative controls in microchambers R₁-R₁₂ at t=0 h and 10 h. The black dots show the yrKHK cell growth in microchambers within 10 h culture. FIG. 35B is a photograph showing magnified and juxtaposed microchambers R₁ and R₁₂ of the negative and high (0.7-7 wt %) concentration ranges at time=10 h to visualize and confirm the strong growth of the yrKHK cells regardless of the lack (negative control) or very high concentrations of glucose (0.7 wt % and 7 wt % in microchambers R₁₂ and R₁, respectively). FIG. 35C is a graph of a dose-response curves of indicated yrKHK S. cerevisiae yeast growth in both positive (exposed with glucose) samples and negative controls obtained using the conventional yeast growth approach.

FIGS. 36A-C relate to testing fructose against yrKHK S. cerevisiae yeast strain. FIG. 36A is a photograph showing the yrKHK yeast strain exposed with fructose in the 0.007-7 wt % range plus negative controls in microchambers R₁-R₁₂ at t=0 h and 10 h. FIG. 36B is a photograph showing magnified microchambers R₈-R₁₂, between which the sensitivity of the yrKHK S. cerevisiae yeast strain to fructose occurs. FIG. 36C is a graph showing yrKHK S. cerevisiae yeast growth in both positive (exposed with fructose) samples and negative controls obtained using the conventional yeast growth approach. In FIG. 36A, the yrKHK cells are seeded at t=0 h and the yrKHK cell growth was monitored at 10 h. There was no difference in the yrKHK growth among the negative, low, and medium concentration ranges. However, in the high concentration range (0.7-7 wt %), the yrKHK cell growth gradually stopped as fewer yeast cells (black colonies) were monitored in microchambers R₁-R₁₂ upon increasing fructose concentration. In FIG. 36B, microchambers R₈-R₁₂ at two time-points (t=0 h and 10 h) were juxtaposed to better visualize the changes in yeast growth and how the yeast growth is stopped within the 0.7-1.113 wt % fructose concentration range. In FIG. 36C, dose-response curves of indicated yrKHK S. cerevisiae yeast growth in both positive (exposed with fructose) samples and negative controls obtained using the conventional yeast growth approach (Rao et al., “Rapid Electrochemical Monitoring of Bacterial Respiration for Gram-Positive and Gram-Negative Microbes: Potential Application in Antimicrobial Susceptibility Testing,” Analytical Chemistry 92(6):4266-4274 (2020), which is hereby incorporated by reference in its entirety. Scale bars are 200 μm in all images.

FIGS. 37A-D relate to testing glucose against WT S. cerevisiae. FIGS. 37A and 37B are photographs showing WT yeast strain exposed with glucose in 0.007-7% range plus negative controls in microchambers R₁-R₁₂ at t=0 h and 10 h, respectively. FIGS. 37C and 37D are photographs with magnified views of two microchambers, showing the yeast growth in microchambers after 10 h. FIG. 37C shows WT yeast growth in the smallest and the largest microchambers (R₁ and R₁₂) of the high (0.7-7%) glucose concentration range at time-points t=0 h and 10 h. FIG. 37D shows WT yeast growth in the 4 picked microchambers (R₁, R₄, R₈, and R₁₂) of the high (0.7-7%) glucose concentration range at time-points t=10 h to visualize the WT S. cerevisiae yeast growth regardless of the glucose concentration introduced to the loaded yeast into microchambers.

FIG. 38 provides photographs showing testing glucose against WT S. cerevisiae. WT yeast strain exposed with fructose in 0.007-7% range plus negative controls in microchambers R₁-R₁₂ at t=0 h and 10 h. Wild-type vs. Fructose (outcome: not sensitive).

### DETAILED DESCRIPTION

The present disclosure relates to gradient-based microfluidic circuits, devices, and methods for performing an assay.

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” depends on the particular system under study, and can be readily appreciated by one of ordinary skill in the art.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

One aspect of the present disclosure relates to a microfluidic circuit comprising an inlet port; an outlet port; a main channel fluidically connecting the inlet port and the outlet port; and a series of dead-end microchambers of differing volumes, where each microchamber is individually fluidically connected to the main channel via a side channel.

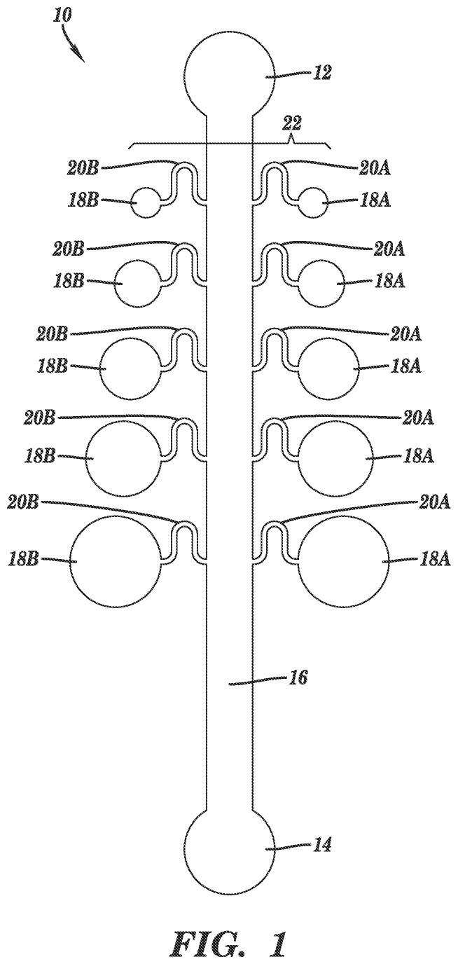

One embodiment of a microfluidic circuit of the present disclosure is illustrated in FIG. 1. FIG. 1 shows a top view of one embodiment of a microfluidic circuit of the present disclosure. Specifically, microfluidic circuit 10 comprises inlet port 12, outlet port 14, and main channel 16. Main channel 16 is fluidically connected to inlet port 12 and outlet port 14 such that fluid may enter inlet port 12, travel through main channel 16, and arrive at outlet port 14.

Microfluidic circuit 10 also has a series of dead-end microchambers 18A and 18B, each of which is individually fluidically connected to main channel 16 via side channels 20A and 20B. In the embodiment illustrated in FIG. 1, microchambers 18A and 18B are mirror images of each other on either side of main channel 16. This particular structure allows a replicate test, one in each pair of microchambers positioned directly across main channel 16 from each other. For example, microchambers 18A and 18B of section 22 of FIG. 1 (i.e., on either side of main channel 16) are identical in size and shape to each other and hold the same volume of fluid to create nearly identical or identical test conditions. The same is true for each of the pairs of microchambers 18A and 18B, which are positioned directly across from each other along main channel 16. However, other arrangements of the microchambers along the main channel are also contemplated. For example, the microchambers need not be mirror images of each other on either side of the main channel. In some embodiments, the microchambers may increase in size on one side of the main channel and decrease in size on the other size of the main channel. According to these embodiments, there may be two equally-sized microchambers on either side of the main channel (e.g., to create a pair of microchambers with identical or nearly identical concentration gradients), but the two equally-sized microchambers do not reside directly across from each other on either side of the main channel. In some embodiments, the microchambers are not arranged in a size or volume gradient along one or both sides of the main channel. According to these embodiments, the microfluidic circuit comprises microchambers of differing volumes, but their positioning along the main channel may not follow an incremental increase (or decrease) in size on one or both sides of the main channel.

In the embodiment illustrated in FIG. 1, side channels 20A and 20B all have identical size, shape, and volume capacity. In addition, in the embodiment illustrated in FIG. 1, each of side channels 20A and 20B comprise a passage having a lower volume capacity than that of main channel 16. Specifically, as illustrated in FIG. 1, side channels 20A and 20B have a channel width narrower than that of main channel 16, although the relative width of the side channels and main channel may vary depending on the particular application. Also, in the embodiment illustrated in FIG. 1, each of side channels 20A and 20B are mirror images of each other, specifically, side channels 20A are mirror images of side channels 20B and vice versa.

In the microfluidic circuits and devices of the present disclosure, the side channels may have any shape, length, channel width, etc., desirable for a particular application, including, in some embodiments, having essentially no length or shape at all, such as when the side channel essentially constitutes nothing more than an opening from the main channel to the microchamber. Thus, in some embodiments, the microchamber may reside directly adjacent the main channel with only an opening between the main channel and microchamber. Such a structure may be suitable in assays involving test agents larger than small molecules (e.g., proteins or enzymes). In some embodiments, it may be useful to have a side channel of greater or lesser length, with some or no bends or curves (e.g., straight), and with greater or lesser channel width. In some embodiments, all of the side channels of a particular microfluidic circuit are identical in their dimensions, and some embodiments, at least one or some of the side channels of a particular microfluidic circuit vary in one or more ways from other side channels of a microfluidic circuit. In some embodiments, microchambers are staggered along the main channel such that the side channels are not directly opposite each other. Being able to vary the dimensions and/or design of the side channel enables flexibility in achieving desired concentration gradients in the microchambers, and allows adaptation of the microfluidic circuit based on the size of test agents (and other variables) in performing assays.

The embodiment illustrated in FIG. 1 shows that side channels 20A and 20B possess a serpentine configuration. As discussed in more detail below, this serpentine configuration assists with flow of fluid from main channel 16 into microchambers 18A and 18B. Other serpentine configurations may also be used, as well as other configurations that create a structural environment for materials and molecules to be transported (such as by diffusion) into microchambers 18A and 18B in a way that achieves the purposes of the microfluidic circuit.

In the embodiment illustrated in FIG. 1, main channel 16 has a linear shape, although main channel 16 may take on other shapes depending on particular application or use of microfluidic circuit 10.

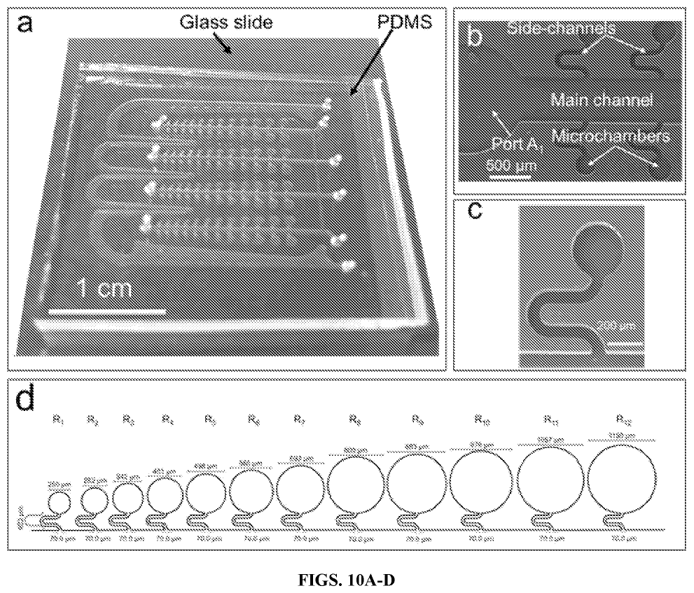

The size of each of the component parts of the microfluidic circuit may vary according to particular application or use. For example, and without limitation, in some embodiments, the side channels may comprise an opening with a width of about 40-100 μm, or 60-80 μm, or about 70 μm, or any particular dimension or range of dimensions therein. In some embodiments, the side channel opening width is 70 μm. The side channels may also comprise a serpentine configuration that forms a switchback configuration (see FIG. 1, side channels 20A and 20B, FIGS. 10C-D, and FIG. 13B) with a switchback length at the shortest distance of about 500-1500 μm, or 880-1100 μm, or about 922 μm, or any particular dimension or range of dimensions therein. In some embodiments, the switchback length at the shortest distance is about 922 μm. In various applications using the microfluidic device, such as applications using proteins, a shorter length side channel may be advantageous. In some embodiments, the side channel length is about 0-5 μm, 5-10 μm, 10-100 μm, or 100-500 μm. In some embodiments, the microchambers have no side channels.

The microchambers may comprise a diameter of between about 200-1500 μm, or 250-1130 μm, or 250, 282, 342, 401, 498, 565, 693, 800, 893, 979, 1057, and/or 1130 μm, or any particular dimension or range of dimensions therein. The microchambers may comprise a diameter larger than 1500 μm or smaller than 200 μm. The inlet port may comprise a diameter of about 500-1500 μm. The outlet port may comprises a diameter of about 500-1500 μm. The inlet and outlet ports may be different sizes, and may have a diameter larger than 1500 μm, or smaller than 500 μm.

As illustrated in FIG. 1, microfluidic circuit 10 comprises five microchambers 18A and five microchambers 18B for a total number of ten microchambers. However, the microfluidic circuit of the present disclosure may comprise any number of microchambers and, in some embodiments, comprises 2, 3, 4, 5, 6, 7, 8, 9, 10, 11, 12, 13, 14, 15, 16, 17, 18, 19, 20, 21, 22, 23, 24 or more than 24 microchambers. In some embodiments, the microfluidic circuit comprises 24 microchambers. In the microfluidic circuit of the present disclosure, microchambers have differing fluidic volumes so as to create a gradient of test volumes to carry out test comparisons. As discussed in more detail below, each microchamber may be filled with a biological substance and then an equal amount of test substance diffuses into the microchamber to create a concentration gradient determined by the size of the microchamber.

In the embodiment illustrated in FIG. 1, microchambers 18A and 18B of microfluidic circuit 10 comprise a circular shape and the diameter of each microchamber 18A and 18B increases as its position increases in distance from inlet port 12. According to this embodiment, microchambers 18A and 18B closest to inlet port 12 have the smallest diameter and hold the smallest volume of fluid and the size and volume of microchambers 18A and 18B increase according to their position moving away from inlet port 12 toward outlet port 14. As discussed in greater detail below, substances tested in microchambers 18A and 18B nearest inlet port 12 will have a greater concentration of test substance than microchambers 18A and 18B positioned further from inlet port 12 and the microchambers furthest from inlet port 12 and closest to outlet port 14 (i.e., microchambers 18A and 18B with the largest size and volume) will create test chambers with the lowest concentration. In other words, in the particular embodiment illustrated in FIG. 1, microchambers 18A and 18B are arranged in size of graduated volumes from lowest to highest from inlet port 12 towards outlet port 14, and are also arranged in equally-sized pairs positioned on either side of main channel 16. Other size and volume arrangements of the microchambers may also be used, including a gradient in the opposite direction (i.e., where the microchambers of the highest volume capacity are nearest the inlet port and the microchambers of the lowest capacity are nearest the outlet port).

The microfluidic circuit of the present disclosure pertains to a fluidic system of ports, channels, and microchambers, all fluidically connected. Since, as discussed in the Examples below, the particular structure of this fluidically connected system enables assays to be performed based on the unique structure of the circuit, the circuit may be used in a variety of contexts or on a variety of platforms. One particular platform where the microfluidic circuit of the present disclosure is useful is a chip-like platform. For example, the microfluidic circuit may be formed into a planar material to create the system of ports, channels, and microchambers, accessible for input or output of fluid only through the ports.

Thus, another aspect of the present disclosure relates to a microfluidic device comprising a support layer; a substrate layer disposed on the support layer; and one or more microfluidic circuits of the present disclosure, where the one or more circuits are disposed within the substrate layer.

In some embodiments, the microfluidic device of the present disclosure includes an aggregation of separate parts, for example, but not limited to, ports, fluid channels, capillaries, joints, chambers, and layers which, when appropriately mated or joined together, form the microfluidic device of the present disclosure. In some embodiments, the microfluidic device may include a top portion, a bottom portion, and an interior portion, one or more of which substantially defines ports, channels, and chambers of the microfluidic device.

In some embodiments, the bottom portion may be a solid support or a substrate that is substantially planar in structure, and which has a substantially flat upper surface. A variety of materials may be used to form the solid support and/or a substrate, which itself is formed on or connected to the solid support. The support and/or substrate materials should be selected based upon their compatibility with known microfabrication techniques, for example, photolithography, 3-D printing, wet chemical etching, laser ablation, air abrasion techniques, injection molding, embossing, and other techniques, or based on the application being used. The support and/or substrate materials are also generally selected for their compatibility with the full range of conditions to which the microfluidic devices may be exposed, including extremes of pH, temperature, salt concentration, and/or application of electric fields, should these be relevant in performing assays using the microfluidic device of the present disclosure.

In some embodiments, suitable support and/or substrate materials include, without limitation, glass, pyrex, glass ceramic, polymer materials, semiconductor materials, and combinations thereof. In some embodiments, the support and/or substrate material may include materials normally associated with the semiconductor industry in which microfabrication techniques are regularly employed, including, e.g., silica based substrates such as glass, quartz, silicon, or polysilicon, as well as other substrate materials, such as gallium arsenide and the like. In the case of semiconductive materials, it will often be desirable to provide an insulating coating or layer, e.g., silicon oxide or silicon nitride, over the support or substrate material, particularly where electric fields are to be applied. In some embodiments, the support layer comprises glass.

Exemplary polymeric materials include, without limitation, plastics such as polymethylmethacrylate (PMMA), polycarbonate, polytetrafluoroethylene (TEFLON™), polyvinylchloride (PVC), polydimethylsiloxane (PDMS), and polysulfone. Other plastics can also be used. Such materials are readily manufactured from microfabricated masters, using well known molding techniques, such as injection molding, embossing or stamping, or by polymerizing the polymeric precursor material within a mold. Such polymeric substrate materials are known for their ease of manufacture, low cost and disposability, as well as their general inertness to most extreme reaction conditions. These polymeric materials may include treated surfaces, for example, derivatized or coated surfaces, to enhance their utility in the microfluidic device or, for example, to provide enhanced fluid direction should that be a needed factor in carrying out assays using microfluidic devices described herein. In some embodiments, the substrate layer comprises PDMS. PDMS is also gas permeable, which allows air to escape into the PDMS substrate from the microchambers during loading of solutions so that the microchambers can be completely filled.

In some embodiments, the material used to build the interior portion, which may at least partially define the microfluidic circuit, is biocompatible and resistant to biofouling. Because the active surface area of the microfluidic device may be only a few μm², the material used to form the interior portion (i.e., the microfluidic circuit) should have a resolution that enables the structuring of both small cross-sectional area channels (on the order of about 2-3 μm width and about 1-2 μm height) and larger cross-sectional area channels (on the order of about 25 to about 500 μm width and/or height, or other dimensions described herein). Several existing materials, widely used for the fabrication of microfluidic circuits, can address these basic needs.

Two categories can be distinguished among them: those based on glasses, such as glass, Pyrex, quartz, etc. (Ymeti et al., Biosens. Bioelectron 20:1417-1421 (2005), which is hereby incorporated by reference in its entirety); and those based on polymers such as polyimide, photoresist, SU-8 negative photoresist, polydimethylsiloxane (PDMS), 3-D printing, silicone elastomer PDMS (McDonald et al., Electrophoresis 21:27-40 (2000), which is hereby incorporated by reference in its entirety), liquid crystal polymer, Teflon, etc.

While glass materials have good chemical and mechanical resiliency, their high cost and delicate processing make them less frequently used for this kind of application. In contrast, polymers have gained wide acceptance as the materials of choice for fluidics (including microfluidics) applications. Moreover, structuring technologies involved in their use, such as bonding, molding, embossing, melt processing, and imprinting technologies, are now well developed (Mijatovic et al., Lab on a Chip 5:492-500 (2005), which is hereby incorporated by reference in its entirety). An additional advantage of polymer-based microfluidic systems is that valves and pumps made with the same material may be readily integrated (Unger et al., Science 288:113-116 (2000), which is hereby incorporated by reference in its entirety).

PDMS and SU-8 resist are particularly well studied as raw materials for the construction of microfluidic systems. While both of them are optically transparent, their mechanical and chemical comportment are strongly disparate. SU-8 is stiffer (Blanco et al., J Micromechanics Microengineering 16:1006-1016 (2006), which is hereby incorporated by reference in its entirety) than PDMS, and so the structuring techniques of these two materials are different. Their chemical properties are an important aspect for the desired application. They both have a hydrophobic surface after polymerization, which can lead to an attachment of the proteins onto the PDMS walls, and can fill the channel in case of small cross-section. Both the surface of PDMS and of SU-8 can be treated with a surfactant or by plasma to become hydrophilic (Nordstrom et al., J Micromechanics Microengineering 14:1614-1617 (2004), which is hereby incorporated by reference in its entirety). The composition of SU-8 can also be modified before its structuring to become hydrophilic after polymerization (Chen and Lee, J Micromechanics Microengineering 17:1978-1984 (2007), which is hereby incorporated by reference in its entirety). Fouling of the channel surface via nonspecific binding is an obvious concern for any microfluidic application. Anecdotal evidence suggests that SU-8 is less prone to this, but it is important to note that chemical treatment methods are also available for improving the performance of PDMS (Lee and Vörös, Langmuir 21:11957-11962 (2004), which is hereby incorporated by reference in its entirety).

Support and/or substrate materials can also be a combination of a glass or Pyrex base and a polymer lid, which together define the microfluidic circuit. In some embodiments, the microfluidic circuit(s) of the microfluidic device of the present disclosure is fabricated as a mold on a silicon wafer to which a layer of PDMS substrate or other material, without limitation, is applied to form the microfluidic device. The PDMS substrate is then peeled off the mold, and applied to a support material, such as glass, and treated to seal the microfluidic circuit(s), as further described herein, and in the Examples. In some embodiments, the “ceiling” of the microfluidic circuit is formed of the contiguous substrate layer, such as when the PDMS material is poured on a silicon wafer mold. In some embodiments, the microfluidic device is fabricated as microscale grooves or indentations formed into the upper surface of the substrate or bottom surface of the polymer lid using microfabrication techniques. In some embodiments, the lower surface of the top portion of the microfluidic device, which top portion (also referred to as top layer) can comprise a second planar substrate, can be overlaid upon and bonded to the surface of the bottom support or substrate, sealing the channels and/or chambers (the microfluidic circuit) of the device at the interface of these two components. Bonding of portions to form a microfluidic device comprising a microfluidic circuit may be carried out using a variety of known methods, depending upon the nature of the support and/or substrate material. For example, in the case of glass supports/substrates, thermal bonding techniques may be used which employ elevated temperatures and pressure to bond one portion of the device to another portion (e.g., a top portion to a bottom portion). Polymeric substrates may be bonded using similar techniques, except that the temperatures used are generally lower to prevent excessive melting of the substrate material. Alternative methods may also be used to bond polymeric parts of the device together, including acoustic welding techniques, or the use of adhesives, for example, UV curable adhesives.

Like the microfluidic circuit, the microfluidic device of the present disclosure is not limited in its physical dimensions and may have any dimensions that are convenient for a particular application. For the sake of compatibility with current laboratory apparatus, microfluidic devices with external sizes of a standard microscope slide or smaller can be easily made. Other microfluidic devices can be sized such that the device fits a standard size used on an instrument, for example, the sample chamber of a mass spectrometer or the sample chamber of an incubator. The microfluidic circuit within the microfluidic device may have any shape, without limitation, such as rectangular, square, oval, circular, or polygonal. The microfluidic circuit, and/or the microchambers and channels that make the microfluidic circuit in the microfluidic device may have square or round bottoms, V-shaped bottoms, flat bottoms, or U-shaped bottoms, without limitation. The shape of the chamber and/or channel bottoms need not be uniform on a particular chip, but may vary. The chambers in the microfluidic device of the present disclosure may have any width-to-depth ratio, which may vary from chamber to chamber. The microchambers wells, channels, and other associated features in the microfluidic device of the present invention may have any volume or diameter which is compatible with the requirements of the sample volume being used. The microchambers or channels can function as a reservoir, a mixer, or a place where chemical or biological reactions take place. In some embodiments, the biological reactions take place in the microchambers.

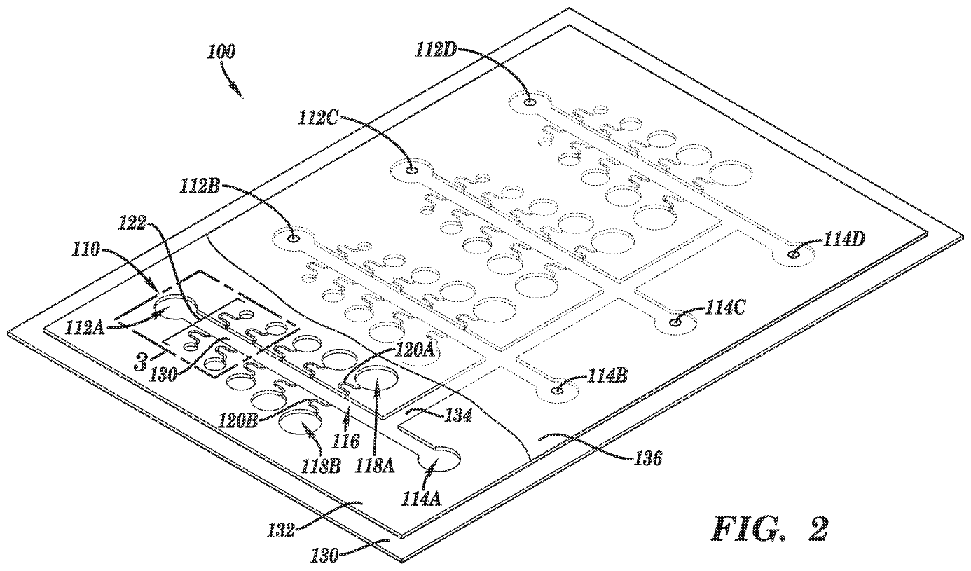

One embodiment of a microfluidic device of the present disclosure is illustrated in FIG. 2. FIG. 2 is a perspective and partial cutaway view of one embodiment of a microfluidic device of the present disclosure. As illustrated, microfluidic device 100 comprises support layer 130. In some embodiments of the microfluidic device of the present disclosure, support layer 130 is a glass slide, although other materials may also be used to support the microfluidic device as discussed above. In some embodiments, the support layer is a planar surface, although non-planar surfaces may also be used as a support layer. In some embodiments, support layer 130 is a non-porous surface. In some embodiments, support layer 130 has some porosity. The porosity may be adjusted for specific applications, such as, without limitation, to let a chemical pass through.

Microfluidic device 100 of FIG. 2 also comprises substrate layer 132, disposed on support layer 130. In some embodiments, substrate layer 132 is made of polydimethylsiloxane (PDMS), although other materials may also be used as discussed above. In some embodiments, substrate layer 132 is made of a material capable of being formed into or comprising a microfluidic circuit. In the particular embodiment illustrated in FIG. 2, substrate layer 132 and top layer 136 (which, according to the embodiment illustrated, is a surface and not a separate layer) are formed as an impression of a silicon wafer mold to create channels and microchambers to form the microfluidic circuit (i.e., microfluidic circuit 110). Specifically, microfluidic circuit 110 is formed into PDMS substrate layer 132 (having top layer (surface) 136) to create a fluidic pathway from inlet port 112A to outlet port 114A. Inlet port 112A and outlet port 114A are fluidically connected via main channel 116. In the particular embodiment illustrated in FIG. 2, support layer 130 is visible in, e.g., inlet port 112A, outlet port 114A, main channel 116, and microchambers 118A and 118B. In other words, substrate layer 132 is open at the bottom such that support layer 130 forms the bottom of microfluidic circuit 110.

Microfluidic circuit 110 of FIG. 2 has a design and structure like that of microfluidic circuit 10 of FIG. 1. Specifically, microfluidic circuit 110 of FIG. 2 comprises inlet port 112A, outlet port 114A, and main channel 116. Main channel 116 is fluidically connected to inlet port 112A and outlet port 114A such that fluid may enter inlet port 112A, travel through main channel 116, and arrive at outlet port 114A.

Microfluidic circuit 110 also has a series of dead-end microchambers 118A and 118B, each of which is individually fluidically connected to main channel 116 via side channels 120A and 120B. In the embodiment illustrated in FIG. 2, microchambers 118A and 118B are mirror images of each other on either side of main channel 116. This particular structure permits a replicate test in each pair of microchambers positioned directly across main channel 116 from each other. For example, microchambers 118A and 118B of section 122 of FIG. 2 (i.e., on either side of main channel 116) are identical in size and shape to each other and hold the same volume of fluid to create similar or identical test conditions. The same is true for each of the pairs of microchambers 118A and 118B directly across from each other along main channel 116.

In the embodiment illustrated in FIG. 2, side channels 120A and 120B all have identical size, shape, and volume capacity. In addition, in the embodiment illustrated in FIG. 2, each of side channels 120A and 120B comprise a passage having a lower volume capacity than that of main channel 116. Specifically, as illustrated, side channels 120A and 120B have a channel width narrower than that of main channel 116, although the relative width of the side channels and main channel may vary. In the embodiment illustrated in FIG. 2, main channel 116 has a linear shape, although main channel 116 may take on other shapes depending on particular application or use of microfluidic circuit 110. Also, in the embodiment illustrated in FIG. 2, each of side channels 120A and 120B are mirror images of each other, specifically, side channels 120A are mirror images of side channels 120B and vice versa. The embodiment illustrated in FIG. 2 also shows that side channels 120A and 120B possess a serpentine configuration. As discussed in more detail below, this serpentine configuration assists with flow of fluid from main channel 116 into microchambers 118A and 119B.

In the embodiment illustrated in FIG. 2, microfluidic circuit 110 is disposed within substrate layer 132, which is contiguous with top layer (surface) 136. Also shown in FIG. 2, a portion of top layer (surface) 136 of microfluidic device 100 has been cut away to reveal microfluidic circuit 110. Underneath top layer 136 of microfluidic device 100 in FIG. 2 are additional microfluidic circuits identical or nearly identical in structure to microfluidic circuit 110, and fluidically connected by a cross channel, i.e., connecting channel 134. In some embodiments, the connecting channel is adjacent to the outlet ports. Inlet ports 112B, 112C, and 112D and outlet ports 114B, 114C, and 114D can be seen in top layer 136 of FIG. 2 and this same structure would be used for microfluidic device 110 of FIG. 2 (i.e., if a complete (non-cutaway) top layer 136 was shown). In some embodiments, the top layer is disposed on the substrate layer. The top layer of the microfluidic device (136 of FIG. 1) can be substrate layer 132 (i.e., such that top layer 136 is the top surface of substrate 132), or can be any of the substrates described above. In some embodiments, the top layer comprises PDMS.

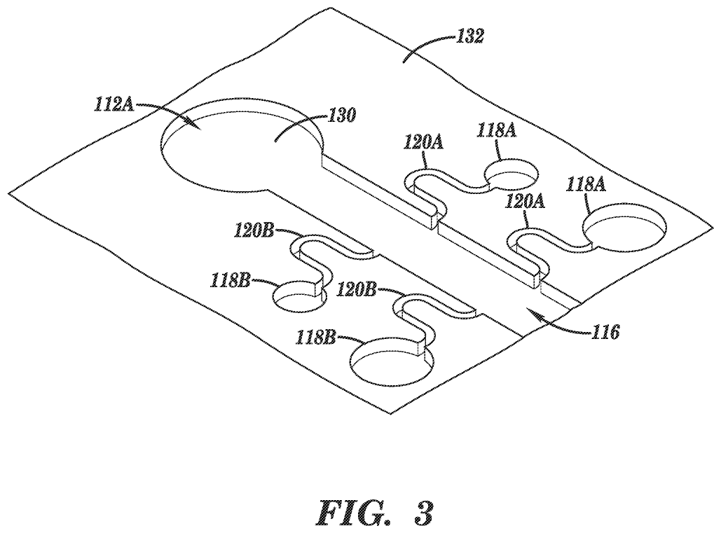

Expanded portion 2 of microfluidic device 100 is illustrated in FIG. 3 to show depth and side walls of microfluidic circuit 110.

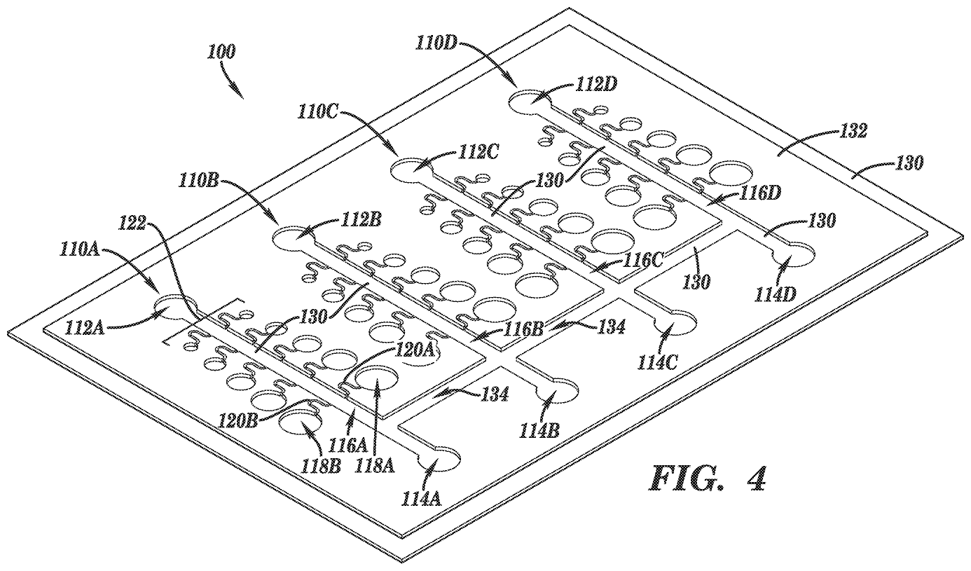

A perspective view of microfluidic device 100 shown in FIG. 2 (i.e., with top layer 136 cut away) is illustrated in FIG. 4. As illustrated, microfluidic device 100 comprises four identical or nearly identical microfluidic circuits 110A, 110B, 110C, and 110D formed within substrate 132 and supported underneath, and having a bottom wall formed by, support layer 130.

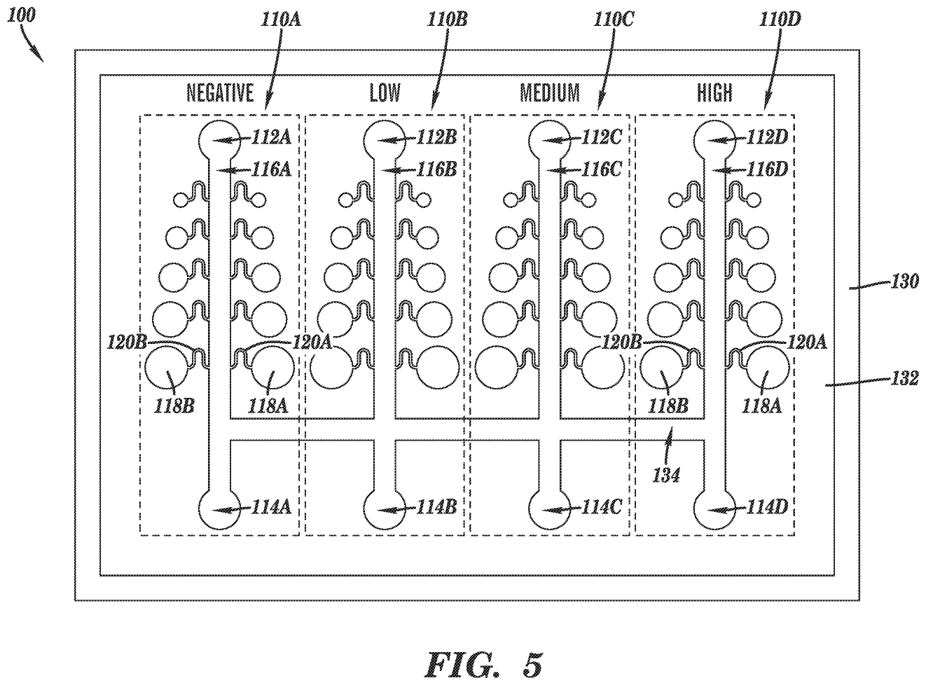

In the embodiment of microfluidic device 100 illustrated in FIG. 4, microfluidic device 100 comprises more than one (i.e., four) microfluidic circuits, including microfluidic circuits 110A, 110B, 110C, and 110D, each of which is essentially identical to each other. Each of microfluidic circuits 110A, 110B, 110C, and 110D is disposed within substrate layer 132. In addition, microfluidic circuits 110A, 110B, 110C, and 110D are connected via a cross channel, specifically, connecting channel 134, which connects each of main channels 116A, 116B, 116C, and 116D. While the embodiment illustrated in FIG. 4 shows four microfluidic circuits (110A, 110B, 110C, and 110D) in microfluidic device 100, any number of microfluidic circuits may be combined to form a microfluidic device of the present disclosure, including one, two, three, four, five, six, or more microfluidic circuits, as needed for any relevant application. In some embodiments, the microfluidic device comprises at least two microfluidic circuits disposed within the substrate layer. In some embodiments, the microfluidic device comprises at least 3, 4, 5, 6, or more than 6 microfluidic circuits disposed within the substrate layer. The particular structure shown in FIG. 4 with four microfluidic circuits (110A, 110B, 110C, and 110D) allows the formation of a microfluidic device where a high, medium, low, and negative (control) gradient can be used in performing an assay using the microfluidic device, as described with reference to FIG. 5.

FIG. 5 is a top view of the cross-sectional microfluidic device of FIG. 4. As illustrated, four microfluidic circuits are shown in substrate layer 132, including microfluidic circuits 110A, 110B, 110C, and 110D, labeled “HIGH”, “MEDIUM”, “LOW”, and “NEGATIVE” to describe the relative concentration of small-molecule test substance introduced into each of inlet ports 112A, 112B, 112C, and 112D to create a high dose concentration in microfluidic circuit 110A, a medium dose concentration in microfluidic circuit 110B, a low dose concentration in microfluidic circuit 110C, and a negative (control) concentration in microfluidic circuit 110D. These four gradients are in addition to the concentration gradients created by differently sized microchambers 118A and 118B associated with each of microfluidic circuits 110A, 110B, 110C, and 110D. These gradients are discussed in more detail in the Examples below.

Movement of fluids into and throughout the microfluidic device of the present disclosure can be controlled manually by introducing solutions into the inlet ports and by diffusion, or by pump connected to one or more inlet ports. Alternatively, the introduction of fluids into and throughout the microfluidic device can be controlled automatically using an operating system programmed to regulate the timing of one or more pipette-like dispersion system, and/or one or more valves responsible for regulating the introduction of fluid (e.g., first reagent, second reagent, isolating solution, etc.). An opening can be introduced through the top layer and substrate material at the inlet and outlet ports for this purpose with a tissue culture puncher, as one non-limiting example (see FIG. 2, 112B-D and 114B-D). In some embodiments, a tissue culture punch of 1 mm is used. In other embodiments, a larger or smaller opening can be made.

Because, as discussed below, one embodiment of methods of using the microfluidic device of the present disclosure is sequential in nature, various systems associated with the microfluidic device may be automated and associated with software that runs on a computer and is easily programmable and modifiable, although one appeal of the microfluidic device of the present disclosure is that it is simple to operate manually by simply introducing a fluid into a microfluidic circuit via an inlet port, and permitting solutions to diffuse throughout the microfluidic circuit, including into microchambers to create gradients by simple diffusion. However, computers in microfluidic systems could also be used to control system processes and receive signals for interpretation. For example, the computer can control a robotic sub-system that retrieves samples or reagents from storage as needed. The computer can control specimen stations to designate the order of drawing samples and reagents for receipt into the microfluidic device. Pressure differentials and electric potentials can be applied to microfluidic devices by the computer through computer interfaces known in the art, thereby controlling pump devices and valves to regulate the flow of reagents into and out of the system, although these are not necessary in the carrying out assays using the microfluidic device of the present disclosure. The computer can be a separate sub-system, it can be housed as an integrated part of a multi-assay instrument, or dispersed as separate computers in modular subsystems.

A computer system for controlling processes and interpreting detector signals can be any known in the art. The computer can also include a software program, which, for example, is useful for correlating, analysis, and evaluation of detector signals, evaluation of the detector signals to quantify activity, etc. The computer can be in functional communication with the one or more valves controlling the inflow and outflow of fluids, flow rate controllers to control the rate and direction of flow inside the microfluidic device. The computer can also control power circuits, control mechanical actuators, receive the information through communication lines, store information, interpret detector signals, make correlations, etc.

Systems including the microfluidic device of the present disclosure can include, e.g., a digital computer with data sets and instruction sets entered into a software system to practice the assay methods described herein. The computer can be a personal computer with appropriate operating systems and software control, or a simple logic device, such as an integrated circuit or processor with memory, integrated into the system. Software for interpretation of detector signals is available, or can easily be constructed by one of skill using a standard programming language such as Visualbasic, Fortran, Basic, Java, or the like.

The microfluidic device of the present disclosure can be in fluidic contact with variety of specimen manipulation stations. These specimen stations can be, for example, autosamplers, such as sample carousels holding multiple small molecule libraries in a circular tray that can be rotated sequentially or randomly to align the library containers with one or more pipettors. The pipettors can be on actuated arms that can dip the pipettor tube into the specimen for sampling or delivery.

In some embodiments, the samples or reagents are of very small volume, for example, as is typical of many molecular libraries. Sampling from such libraries, e.g., on microwell plates or microarray slides, is typically accomplished with robotic systems that precisely position the pipettor tip in the micro specimen. In embodiments where the library members are retained in dehydrated form, it can be convenient to sample by ejecting a small amount of solvent from the pipettor to dissolve the specimen for receipt into the microfluidic device of the present disclosure.

Reagents can be any composition useful in assays suitable for being carried out with the microfluidic device of the present disclosure, for example, chemicals or biomolecules capable of interacting with target molecules, controlling the reaction conditions, or generating a detectable signal. Reagents are typically one or more molecules in a solution that can flow into contact with the target in a chamber. Reagents can include a chromophore that reacts with the target to provide a changed optical signal.

Within the microfluidic device are microchambers where the first reagent and second reagent come into contact in the particular concentration defined by the size of the microchamber. These microchambers can also be configured to provide conditions amenable to provide a detectable signal resulting from the contact between targets, if necessary.

Microfluidic devices can also have detection regions that can be monitored by detectors which detect the signals, for example, resulting from cellular growth or density, contact of targets, a signal from a reagent that has reacted with a sample analyte, the absence of a detectable signal (interpretable, e.g., as the absence of sample analyte at a level adequate to generate a signal above the sensitivity of the detector), a signal amplitude related to a quantity of a sample analyte, and/or the like. The detection regions are, in some embodiments, the microchambers of the microfluidic circuit. For example, detector regions can incorporate sensors such as pH electrodes and/or conductivity meter electrodes. Detection regions can comprise one or more microchambers transparent to certain light wavelengths so that light signals, such as, absorbance, fluorescent emissions, chemoluminescence, and the like, can be detected. Detectors can be located in the microfluidic device, or proximate to the device, in an orientation to receive signals resulting from the sample contact with the reagent. Detectors can include, e.g., a nucleic acid sequencer, a fluorometer, a charge coupled device, a laser, a photo multiplier tube, a spectrophotometer, scanning detector, microscope, or a galvo-scanner. Signals detected from interactions of reagents and samples can be, e.g., absorbance of light wavelengths, light emissions, radioactivity, conductivity, refraction of light, etc. The character of signals, such as, e.g., the amplitude, frequency, duration, counts, and the like, can be detected.

Detectors can detect signals from detector regions described by physical dimensions, such as a point, a line, a surface, or a volume from which a signal can emanate. In some embodiments, the detector can scan an image of a surface or volume for signals resulting from interactions of reagents and samples. For example, a detector can contemporaneously image multiple parallel microchambers carrying reaction mixtures from multiple analyses to detect results of several different assays at once.

The detectors can transmit detector signals that express characteristics of resultant signals received. For example, the detector can be in communication with an output device, such as an analog or digital gage, that displays a value proportional to a resultant signal intensity. The detector can be in communication with a computer through a data transmission line to transmit analog or digital detector signals for display, storage, evaluation, correlation, and the like.

In some embodiments, detecting an interaction between the first reagent solution and the second reagent solution comprises detecting a visual signal. In some embodiments, detecting an interaction between the first reagent solution and the second reagent solution comprises detecting a fluorescent signal. In some embodiments, detecting an interaction between the first reagent solution and the second reagent solution comprises detecting a colorimetric signal. In some embodiments, detecting an interaction between the first reagent solution and the second reagent solution comprises detecting a spectrophotometric signal.

A further aspect of the present invention relates to kits that include a microfluidic device of the present disclosure and, optionally, one or more pools of reagents for carrying out assays suitable to the microfluidic device described herein.

Another aspect of the present disclosure relates to a method for performing an assay. This method involves loading a first reagent solution into the inlet port of a microfluidic device of the present disclosure. A second reagent solutions is loaded into the inlet port, and then an isolating solution is loaded into the inlet port. The method further involves detecting an interaction between the first reagent solution and the second reagent solution in one or more of the microchambers.

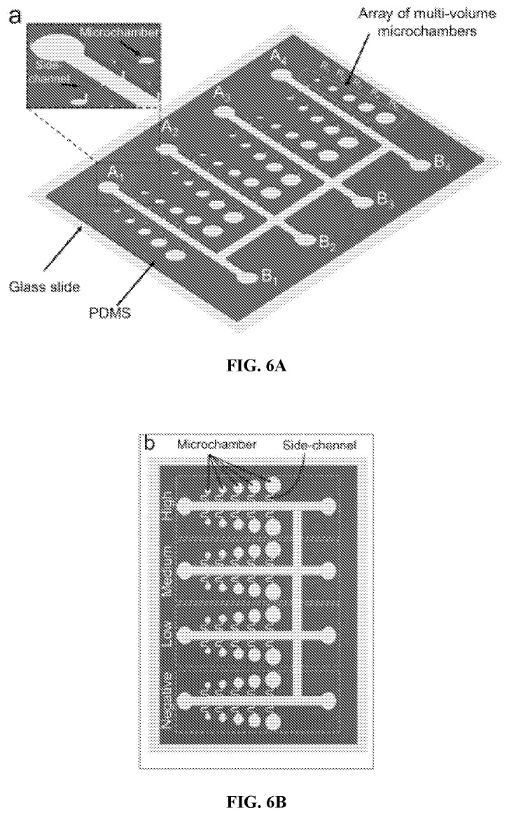

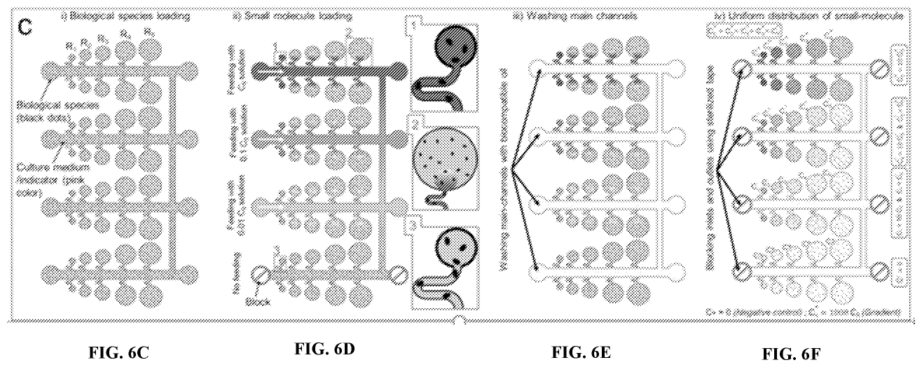

Turning now to FIGS. 6A-F, illustrated is one embodiment of a method of performing an assay using the microfluidic circuit and/or device of the present disclosure. In the particular embodiments illustrated in FIGS. 6A-F, this method is carried out by carrying out a series of “loading” steps, including: FIG. 6C: loading a first reagent solution (e.g., a biological species) into the inlet port of a microfluidic device of the present disclosure until a uniform concentration of the first reagent solution is achieved throughout all microfluidic circuits in the microfluidic device; FIG. 6D: loading a second reagent solution (e.g., comprising a small molecule to be tested against the first reagent solution in different concentrations) into the inlet port. The second reagent solution is transported or permitted to diffuse throughout the microfluidic circuit from the injection site (i.e., inlet port). In some embodiments, the second reagent solution is loaded into the microfluidic circuits in three different concentrations, respectively, at three orders of magnitude (C₀, 0.1 C₀, and 0.01 C₀) represented by degree of shading in each of the microfluidic circuits; FIG. 6E: loading an isolating solution into the inlet ports of each of the microfluidic circuits to stop the loading of the second reagent solution into the microchambers (e.g., by washing the main-channels with a biocompatible oil to isolate the microchambers containing the biological species and small-molecules); and, optionally, FIG. 6F: blocking the inlet ports and outlet ports of the microfluidic circuits with, e.g., sterilized medical tape and letting the loaded small molecules uniformly distribute in the microchambers. As a result, the smallest and largest microchambers in each row feature the highest and lowest concentrations of the small-molecule in the low, medium, and high ranges. Moreover, the smallest microchambers of the low and medium ranges feature identical concentrations as the largest microchambers of the medium and high ranges, respectively, ensuring there is continuity within the concentrations tested.

Thus, in carrying out the method of the present disclosure, the pattern illustrated in FIGS. 6C-6F may be carried out, which involves (i) loading a biological species to diffuse equally through the microfluidic circuit(s) of the microfluidic device; (ii) loading small molecule into each of inlet ports of all but one of the microfluidic circuits of the microfluidic device, each at a different concentration, to create a second layer of concentration gradients in the microfluidic device; (iii) washing the main channel of each microfluidic circuit step-by-step after the introduction of a small molecule into the circuit to prevent transport or diffusion of the small molecule solution out of the microchamber; and (iv) allowing uniform distribution of the small molecule solution throughout the microchambers to create concentration gradients in each of the microfluidic circuits.

Other variations may also be used based on loading of a biological species, loading of a small molecule test agent, loading an isolating solution, and achieving distribution of the small molecule test agent to detect effectiveness of the small molecule against the biological species at the various concentrations created in the microfluidic device.

In some embodiments, when loading the first reagent into the microfluidic circuit, the first reagent fills the microchambers. Creating an even distribution of first reagent throughout the microfluidic circuit ensures that true concentration gradients of the second reagent are achieved. In some embodiments, the structure of the microchambers or side-channels may be changed to allow non-uniform reagents into the microchambers for further biological or non-biological assays.

In some embodiments, the second reagent solution is loaded into the inlet port(s) of the microfluidic device and allowed to diffuse throughout the circuit and into the microchambers. In some embodiments, a portion of the second reagent solution diffuses into the microchambers, thereby forming a concentration gradient of the second reagent solution within the microchambers from the inlet port to the outlet port. In some embodiments, a third reagent may be loaded prior to the isolating solution. In some embodiments, a fourth reagent may be loaded prior to the isolating solution. In some embodiments, a fifth, a sixth, a seventh, an eighth, or more than eighth reagent may be loaded prior to the isolating solution.

In some embodiments in carrying out the methods described herein, one microfluidic circuit is loaded with the second reagent at a time. In some embodiments in carrying out the methods described herein, one microfluidic circuit is loaded with the second reagent while the inlet and outlet ports of other microfluidic circuits of a microfluidic device are blocked, sealed, or closed. A blocking element may be used for blocking, sealing, or closing inlet and outlet ports of the microfluidic device of the present application. Blocking, sealing, or closing inlet and outlet ports may be carried out by any suitable means including, without limitation, by sealing the ports with tape, inserting a block agent into the ports, or fitting tubing into the ports and sealing the tubing. Blocking may be carried out reversibly to allow access to the inlet and outlet ports at various times during the methods described herein. Inlet and outlet ports may be open or blocked as needed for the loading of the first reagent, the second reagent, and the isolating solution(s). In some embodiments, the microfluidic inlet port and outlet port comprise a blocking element. Similar methods with different loading arrangement may be used depending on the application.

In carrying out the methods of the present application, the isolating solution is used to prevent transport or diffusion of the first reagent solution and second reagent solution from the microchambers. Suitable isolating solutions include, without limitation, a biocompatible oil such as glycerol, vegetable oil, and silicon oil. Other substances may be used, including, for example, those that are more viscous than water and/or are hydrophobic.

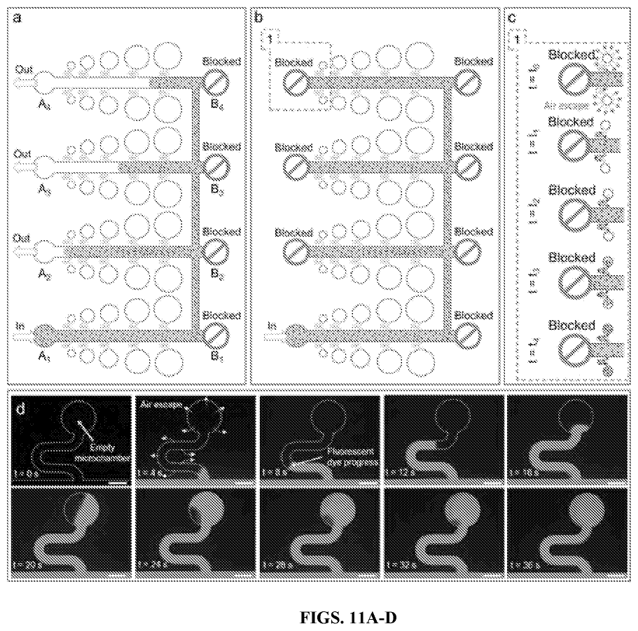

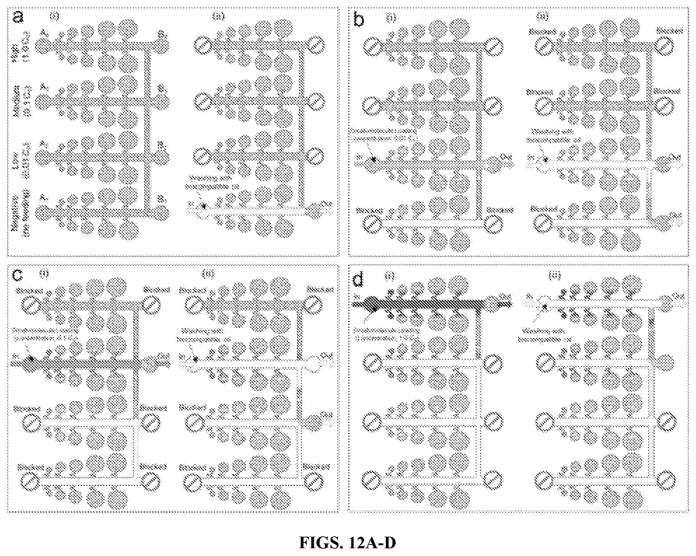

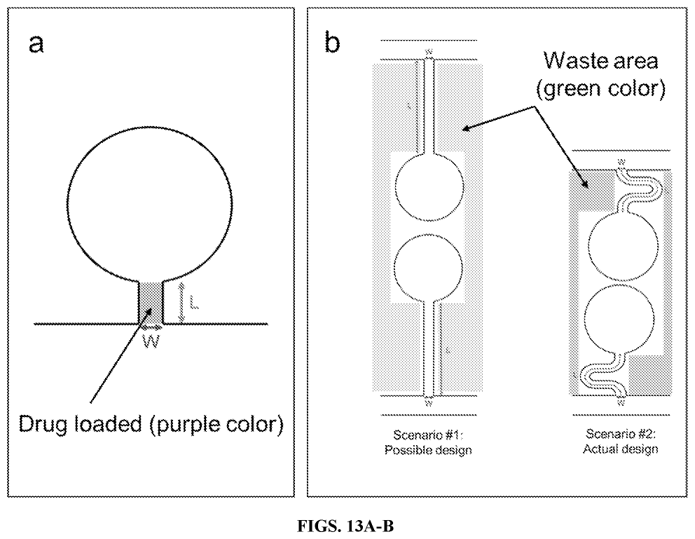

In some embodiments in carrying out the methods described herein, the microfluidic device is loaded with a first reagent solution into the inlet port of one or more of the microfluidic circuits, while the outlet ports are blocked (FIG. 11A). All inlet and outlet ports can then be blocked except for one inlet port in order to complete the loading of the first reagent (FIG. 11B). Due to the pressure of the solution flow, entrapped air in the microchambers escapes through the PDMS walls, resulting in a uniformly loaded first reagent throughout the device (FIG. 11C). Inlet and outlet ports can be used interchangeably for this process. After the first reagent is loaded, the negative control microfluidic circuit can then be washed with an isolating solution to prevent the microchambers from receiving any further solutions (FIG. 12A). Either the inlet port or the outlet port for the negative control microfluidic circuit can be used to load the isolating solution. In some embodiments, the inlet and outlet ports for the other microfluidic circuits may be blocked during this process (FIG. 12A).

In further embodiments in carrying out the methods described herein, the microfluidic device is then loaded with a second reagent solution for a specified time, followed by an isolating solution, as shown in FIGS. 12B-D, and as discussed in the Examples. In some embodiments, the isolating solution prevents transport or diffusion of the first reagent solution and the second reagent solution from the microchambers. In some embodiments, the inlet and outlet ports of circuits that are not being loaded with the second reagent solution or the isolating solution are blocked. The timing of loading of the second solution in order to produce a gradient concentration profile (GCP) in the microchambers is determined by the small-molecule diffusion coefficients and diffusion time of the small molecule. The loading time can be determined empirically, or calculated as shown in the Examples. Exemplary loading times for various chemicals are shown in Tables 1-3.

The microfluidic circuits and devices of the present disclosure can be used to perform a variety of assays. Accordingly, the first reagent may be any number of possible substances, including, for example and without limitation, a biological sample. In some embodiments, the biological sample comprises a prokaryotic cell or prokaryotic cell component. In some embodiments, the biological sample comprises a eukaryotic cell or eukaryotic cell component.

Suitable second reagent solutions may comprise an antimicrobial compound. Thus, the method of the present disclosure may be carried out to perform an assay testing the ability of a compound (e.g., small molecule) to act as an antimicrobial compound. Moreover, the method may be carried out to determine an effective or ideal concentration at which an antimicrobial compound may be used to achieve its desired effect.

Antimicrobial compounds are well known in the art. Non-limiting examples of antimicrobial compounds that may be used in the assay method of the present disclosure include, without limitation, Actinomycin D, Actinonin, Aculeacin A, Acycloguanosine (Aciclovir), Adenine 9-β-D-arabinofuranoside (Vidarabine), Alamethicin, L-Alanyl-L-1-aminoethylphosphonic acid (Alafosfalin), Albendazole (Methyl 5-(propylthio)-2-benzimidazolecarbamat), 17-(Allylamino)-17-demethoxygeldanamycin (Tanespimycin), Amastatin, Amikacin, 7-Aminoactinomycin D (7-ADD), 7-Aminocephalosporanic acid (7-ACA), 7-Aminodesacetoxycephalosporanic acid (7-ADCA), (+)-6-Aminopenicillanic acid (6-APA), Amoxicillin, Amphotericin B (Fungizone), Ampicillin (D-(—)-α-Aminobenzylpenicillin), Anhydroerythromycin A, (Erythromycin, or 2BR5PL6H3), Anisomycin (or, Flagecidin), Aphidicolin, Apicidin, Apoptolidin (FU 40A), Apramycin (Nebramycin II), Artesunate, Ascochlorin (Ilicicolin D, NSC 287492), Ascomycin (KD4185000), Azacitidine (Ladakamycin), Azithromycin, Azlocillin (D-α-([Imidazolidin-2-on-1-yl]carbonylamino)benzylpenicillin), Bacitracin, Bafilomycin A₁ (4730700), Bafilomycin B₁, Bestatin (Ubenimex), Bithionol, Blasticidine (Blasticidin S), Borrelidin, Brefeldin A (Ascotoxin, BFA, Cyanein, Decumbin), Caerulomycin A (Carulomycin A, Cerulomycin), Calcium ionophore III (ANTIBIOTIC A 23187, Calimycin), (S)-(+)-Camptothecin, (Camptothecin), Carbenicillin (α-Carboxybenzylpenicillin), Cefaclor, Cefalexin, Cefazolin, Cefixime, Cefmetazole, Cefoperazone, Cefotaxime ((Z)-Cefotaxime), Cefmetazole, Cefoperazone, Cefsulodin (Ulfaret), Ceftazidime, Ceftriaxone, Cephalexin, Cephalomannine (NSC 318735), Cephalothin (Cefalotin), Cephradine (Cefradine), Cercosporin, Cerulenin, Chloramphenicol, Chlorhexidine, Chloroquine, Chlortetracycline, Chromomycin A₃, Chrysomycin A (MFCD07370133), Chrysomycin B (MFCD07370132), Cinoxacin, Clarithromycin, Clindamycin (Cleocin), Clofazimine, Clotrimazole, cloxacillin, Colistin, Concanamycin A (Folimycin), Cordycepin (3′-Deoxyadenosine), Coumermycin A₁, Cryptotanshinone (Tanshinone C), Cycloheximide (Actidione, Naramycin A), D-Cycloserine, Cyclosporin A (Antibiotic S 7481F1, Cyclosporine), Cytochalasin D (Zygosporin A, 1632828), Cytochalasin B (Phomin), Dacarbazine ((E)-Dacarbazine), Daptomycin, Daunorubicin (Daunomycin), 10-Deacetylbaccatin III, Demeclocycline, 1-Deoxymannojirimycin, Dichlorophene, Dicloxacillin, Difloxacin, Dihydrostreptomycin, Dimetridazole, Dirithromycin, Doxorubicin, Doxycycline, Duramycin, Econazole, Embelin (Embelic acid, Emberine), Emetine, Enrofloxacin (Baytril), Erythromycin (E-Mycin, Erythrocin), Ethambutol ((+)-S,S-Ethambutol), Etoposide, Florfenicol (Aquafen, Nuflor), Flubendazol (Flumoxanal), Fluconazole, Flumequine, 5-Fluorocytosine (Flucytosine), Flurbiprofen, Formycin A (Adenosine, Formycin, NSC 102811), Fumagillin, Furazolidone, Fusaric acid (5-Butylpicolinic acid), Fusidic acid, G 418, Ganciclovir, Gatifloxacin, Gentamicin, Gliotoxin, gramicidin s, Griseofulvin, Herbimycin A, Honokiol, 8-Hydroxyquinoline, 4-Hydroxytamoxifen ((Z)-Afimoxifene), Hygromycin B (WK2130000), Ikarugamycin, Imipenem, Ionomycin, Irgasan, Itraconazole, Ivermectin Bla, Josamycin, K-252a, Kanamycin, Ketoconazole, Kirromycin (mocimycin), Lactoferricin B (metallibure), Leptomycin A, Leptomycin B, Levamisol (Levamisole), Levofloxacin, Lincomycin, Lomefloxacin, Lysobactin, Magainin I, Mebendazole, Meclocycline, N-Methyl-1-deoxynojirimycin (1524564), Metronidazole, Mevastatin, Miconazole, Minocycline, Mithramycin A (Plicamycin), Mitomycin C (Mitomycin), Monensin, Moxalactam (Latamoxef), Mupirocin, Myxothiazol, Nafcillin, Naftifine, Nalidixic acid, Narasin, Neocarzinostatin (Holoneocarzinostatin, NCS, NSC-69856, Zinostatin), Neomycin, Netilmicin, Netropsin (Congocidin, Sinanomycin), Niclosamide, Nigericin (Antibiotic K178, Antibiotic X464, Azalomycin M, Helexin C, Polyetherin A), Nikkomycin, Nitrofurantoin (Furadoxyl, Nitrofurantoine), Nonactin (Ammonium ionophore), Norfloxacin, Novobiocin, Nystatin (Fungicidin, Mycostatin), Ochratoxin A, Ofloxacin, Oligomycin A, Oxacillin, Oxolinic acid, Oxytetracycline, Paclitaxel, Paromomycin, Patulin, PD 404,182, Pefloxacin, Penicillin G (Benzylpenicillin), Pentamidine, Phenazine, Phleomycin (UNII:BN3E7WJN9X), Phosphomycin (Fosfomycin), Pimaricin (Natamycin), Pipemidic acid, Piperacillin, Pirarubicin (THP), Polymyxin B, Praziquantel, PUROMYCIN, Pyrazinecarboxamide (Pyrazinamide, Pyrazinoic acid amide), Pyronaridine, Pyrrolnitrin, Quinine, 8-Quinolinol (8-Hydroxyquinolin, Oxine), Radicicol, Rapamycin (Sirolimus), Reveromycin A (MFCD00912537), Ribavirin, Ribostamycin, Rifabutin (Ansamycin, Ansatipine (Farmitalia), LM-427, Mycobutin (Farmitalia)), Rifampicin (Rifampin, Rifamycin AMP), Rifapentine (DL 473), Rifaximin (Rifacol), Roxithromycin, Salinomycin, Sisomicin, Sorbic acid, Sordarin, Sparfloxacin, Spectinomycin, Spergualin, Spiramycin, Staurosporine (Staurosporine), Streptomycin, Streptonigrin (Bruneomycin, Nigrin), Streptozocin (Streptozotocin), Prothionamide, and Monensin.

Suitable second reagent solutions may also comprise an anticancer compound or drug. According to some embodiments, when the second reagent solution comprises an anticancer compound or drug, the first biological sample of the first reagent solution is a cancer cell or a component of a cancer cell. Thus, the method of the present disclosure may be carried out to perform an assay testing the ability of a compound (e.g., small molecule) to act as an anticancer drug. Moreover, the method may be carried out to determine an effective or ideal concentration at which an anticancer compound may be used to achieve its desired effect. Anticancer compounds are well known in the art. Non-limiting examples of anticancer compounds that may be used in the assay method of the present disclosure include, without limitation, Abemaciclib (Verzenio), Abiraterone acetate (Zytiga), Acalabrutinib, Afinitor (Everolimus), Aldara (Imiquimod), Alectinib, Alemtuzumab, Alimta (Pemetrexed Disodium), Aliqopa (Copanlisib Hydrochloride), Aloxi (Palonosetron Hydrochloride), Alunbrig (Brigatinib), Ameluz (Aminolevulinic Acid), Amifostine, Anastrozole, Apalutamide, Aprepitant, Arimidex (Anastrozole), Aromasin (Exemestane), Arranon (Nelarabine), Axitinib, Azacitidine (Vidaza), Azedra, Beleodaq (Belinostat), Bendamustine Hydrochloride, Bexarotene (Targretin), Bicalutamide (cosodex), BiCNU (Carmustine), Binimetinib, Bortezomib (Velcade), Bosutinib, Braftovi (Encorafenib), Brigatinib, Busulfan, Cabazitaxel, Calquence (Acalabrutinib), Campath (Alemtuzumab), Camptosar (Irinotecan Hydrochloride), Capecitabine (Xeloda), Carac (Fluorouracil—Topical, Tolak), Carfilzomib (Kyprolis), Carmustine, Ceritinib (Zykadia), Cerubidine (Daunorubicin Hydrochloride), Chlorambucil, Cladribine, Clofarabine, Cobimetinib, Copanlisib Hydrochloride, Copiktra (Duvelisib), Cosmegen (Dactinomycin), Crizotinib (Xalkori), Cyclophosphamide, Cytarabine (Tarabine PFS), Dabrafenib (Tafinlar), Dacarbazine, Dacogen (Decitabine), Dacomitinib (Vizimpro), Dasatinib, Daunorubicin Hydrochloride, Decitabine, Degarelix, Dexamethasone, Dexrazoxane Hydrochloride (Totect, Zinecard), Docetaxel (Taxotere), Doxorubicin, Duvelisib, Leuprolide Acetate, Ellence (Epirubicin Hydrochloride), Eltrombopag Olamine, Emend (Aprepitant), Enasidenib Mesylate, Enzalutamide (Xtandi), Epoetin Alfa, Eribulin Mesylate, Erivedge (Vismodegib), Erleada (Apalutamide), Erlotinib Hydrochloride (Tarceva), Ethyol (Amifostine), Etopophos (Etoposide Phosphate), Everolimus, Evista (Raloxifene Hydrochloride), Evomela (Melphalan Hydrochloride), 5-FU (Fluorouracil Injection), Fareston (Toremifene), Farydak (Panobinostat), Faslodex (Fulvestrant), FEC, Femara (Letrozole), Firmagon (Degarelix), Fludarabine Phosphate, Flutamide, Folotyn (Pralatrexate), Fusilev (Leucovorin Calcium), Gefitinib, Gemcitabine Hydrochloride, Gilotrif (Afatinib Dimaleate), Gleevec (Imatinib Mesylate), Gliadel Wafer (Carmustine Implant), Goserelin, Acetate (Zoladex), Granisetron, Halaven (Eribulin Mesylate), Hemangeol (Propranolol Hydrochloride), Hycamtin (Topotecan Hydrochloride), Hydrea (Hydroxyurea), Ibrance (Palbociclib), Ibritumomab Tiuxetan (Zevalin), Ibrutinib, Iclusig (Ponatinib Hydrochloride), Idarubicin Hydrochloride, Idelalisib (Zydelig), Ifex (Ifosfamide), Imiquimod, Ipilimumab (Yervoy), Istodax (Romidepsin), Ivosidenib (Tibsovo), Ixabepilone, Ixazomib Citrate, Ruxolitinib phosphate, Kisqali (Ribociclib), Lanreotide Acetate, Larotrectinib Sulfate (Vitrakvi), Lenalidomide, Lenvatinib Mesylate, Lomustine, Lorbrena (Lorlatinib), Lupron (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Mercaptopurine, Methotrexate, Midostaurin, Mitomycin C, Mitoxantrone Hydrochloride, Mozobil (Plerixafor), Mustargen (Mechlorethamine Hydrochloride), Myleran (Busulfan), Navelbine (Vinorelbine Tartrate), Nelarabine, Neratinib Maleate, Neulasta (Pegfilgrastim), Nexavar (Sorafenib Tosylate), Nilandron (Nilutamide), Nilotinib (Tasigna), Ninlaro (Ixazomib Citrate), Odomzo (Sonidegib), Omacetaxine Mepesuccinate, Osimertinib (Tagrisso), Paclitaxel (Taxol), PAD, Palbociclib, Palonosetron Hydrochloride, Panobinostat, Pazopanib Hydrochloride (Votrient), Pegfilgrastim (Zarxio), Pomalidomide, Prednisone, Procarbazine Hydrochloride, Promacta (Eltrombopag Olamine), Purinethol, Raloxifene Hydrochloride, Regorafenib, Ribociclib, Rheumatrex (Methotrexate, Trexall), Rolapitant Hydrochloride, Romidepsin, Rubidomycin (Daunorubicin Hydrochloride), Rydapt (Midostaurin), Sancuso (Granisetron), Somatuline Depot (Lanreotide Acetate), Sonidegib, Stivarga (Regorafenib), Tabloid (Thioguanine), Temodar (Temozolomide), Temsirolimus (Torisel), Thalidomide, Thiotepa, Toremifene, Trabectedin (Yondelis), Treanda (Bendamustine Hydrochloride), Trifluridine and Tipiracil Hydrochloride, Uridine Triacetate (Vistogard), Valrubicin, Vandetanib, Varubi (Rolapitant Hydrochloride), Vemurafenib (Zelboraf), Venclexta (Venetoclax), Vinblastine Sulfate, Vismodegib, Vorinostat (Zolinza), Xospata (Gilteritinib Fumarate), Zofran (Ondansetron Hydrochloride), and Zoledronic Acid (Zometa).

Suitable second reagent solutions may also comprise an antifungal drugs and other small molecules (e.g., for testing on yeast or other fungal species). Thus, the method of the present disclosure may be carried out to perform an assay testing the ability of a compound (e.g., small molecule) to act as an antifungal drug. Moreover, the method may be carried out to determine an effective or ideal concentration at which an antifungal compound may be used to achieve its desired effect. Antifungal compounds are well known in the art. Non-limiting examples of antifungal compounds that may be used in the assay method of the present disclosure include, without limitation, D-fructose, Glucose, Galactose, Antimycin, Bleomycin, 5-Bromo-5-nitro-1,3-dioxane, Cinnamycin, Fengycin (Plipastatin), Filastatin, Filipin, Gentian Violet, Sinefungin, Kasugamycin, Magnolol (2,2′-Bichavicol, 5,5′-Diallyl-2,2′-biphenyldiol), Oligomycin (Oligomycin A), Surfactin, Terconazole, Thiabendazole (2-(4-Thiazolyl)benzimidazole), Thiolutin, Thymol (5-Methyl-2-isopropylphenol), Tioconazole, Tolnaftate, Tubercidin, Terbinafine, Ketoconazole, Fluconazole, Itraconazole, Voriconazole, Caspofungin, and Flucytosine.

In some embodiments in carrying out the methods described herein, the microfluidic device further comprises an in-water bath cavity surrounding the microfluidic circuits. The in-water bath cavity is not fluidically connected to the microfluidic circuits. The in-water bath cavity provides access for a solution such as water, without restriction, to be added through inlet or outlet ports that provide access to the in-water bath cavity, but not the microfluidic circuits (see, e.g., FIGS. 26A-C). Micropillars of substrate material such as PDMS provide support for the in-water bath cavity. In some embodiments, the microfluidic device is placed in an external water bath (“out-water bath” (FIG. 26D)). In further embodiments, the microfluidic device is incubated at a temperature conducive to growth of the biological solution.

The following examples are intended to exemplify the practice of embodiments of the disclosure but are by no means intended to limit the scope thereof.

### EXAMPLES

### Example 1—Materials and Methods

MVM² Device Fabrication. The MVM² device was made following the Microchem Corp. (Newton, MA) instruction for microfluidic device fabrication using soft lithography techniques (Xia et al, “Soft Lithography,” Annual Review of Materials Science 28(1):153-184 (1998); Pajoumshariati et al, “Microfluidic-Based Cell-Embedded Microgels Using Nonfluorinated Oil as a Model for the Gastrointestinal Niche,” ACS Applied Materials & Interfaces 10(11):9235-9246 (2018), Pajoumshariati et al, “A Microfluidic-Based Model for Spatially Constrained Culture of Intestinal Microbiota,” Advanced Functional Materials 28(48):1805568 (2018); and Yaghoobi et al, “Progressive Sperm Separation Using Parallelized, High-Throughput, Microchamber-based Microfluidics,” bioRxiv (2020) doi.org/10.1101/2020.07.31.231373; each of which are hereby incorporated by reference in their entirety). Briefly, SU-8 2050 negative photoresist (Microchem Corp. Newton, MA) was poured on a silicon wafer (ID: 452, UniversityWafer, Boston, MA) and spun-coated at 2100 rpm. Then, the wafer was pre-baked at 65° C. and 95° C. for 3 and 9 min, respectively. The pre-baked SU-8 photoresist was then patterned using a photomask made by CAD/Art Services, Inc. (Bandon, OR) via UV light wavelength with an exposure energy of 120 mJ/cm² at 365 nm. Then, a post-baking step was followed at 65° C. and 95° C. for 2 and 7 min, respectively, to further permanently stabilize the SU-8 photoresist pattern on the silicon wafer. The uncured SU-8 (non-patterned parts of SU-8) was developed and washed by gently soaking the SU-8 patterned silicon wafer in the SU-8 developer (Microchem Corp. Newton, MA) for 10 min. The mixture of PDMS and its curing agent (10:1) was then poured on the SU-8 deposited wafer and baked for 2 h at 65° C. After peeling the PDMS off the patterned wafer, injection holes (1 mm in diameter) were punched and cleaned by a cellophane tape (3M Scotch Magic, MN, USA), followed by bonding of PDMS to a glass slide by applying oxygen-plasma treatment for 1 min. To further stabilize the bonding strength between PDMS and the glass slide, the device was kept at 65° C. in an oven for 12 h for further stabilization.

Bacteria culture medium, strains, growth, and broth microdilution test. Non-selective Mueller-Hinton (“MET”) culture medium was used for all bacterial culture. In detail, a colony of bacteria pre-cultured on an LB agar plate (Corning, NY) was taken from a freshly streaked plate and suspended into 3 mL of MEI broth, and cultured at 37° C. overnight (˜12 h). The concentration of bacterial suspension was adjusted using a UV-Vis spectrophotometer (NanoDrop, Thermo-Fisher; Wilmington, DE). To do this, the overnight culture medium was diluted by 10-fold serial dilutions in non-selective MEI culture medium to find the appropriate final concentration (1×10⁶ CFU/mL) for the antimicrobial susceptibility testing assay.

Yeast culture medium, strains, and growth. Yeast cell growth and standard laboratory manipulations were performed as described (Guthrie and Fink, “Guide to Yeast Genetics and Molecular and Cellular Biology,” Methods in Enzymol. Part C 351 Gulf Professional Publishing (2002), which is hereby incorporated by reference in its entirety). All media used was either minimal medium (YNB; 0.67% yeast nitrogen base without amino acids plus 2% indicated carbon sources) or rich medium (YP; 2% bacto peptone, 1% yeast extract, 2% indicated carbon sources). Additionally, for fluorescence microscopy, low-fluorescence medium was used (standard minimal medium with 2% glucose, except YNB is prepared without riboflavin or folic acid to reduce background fluorescence) (Sheff and Thorn, “Optimized Cassettes for Fluorescent Protein Tagging in Saccharomyces cerevisiae,” Yeast 21:661-670 (2004), which is hereby incorporated by reference in its entirety). The yeast strains used in this study were DBY12000 and DBY12549. DBY12000 is a wild-type yeast strain (i.e., WT) with a genotype of MATa prototrophic HAP1⁺ derivative of FY4. DBY12549 or yrKHK is the mutant of the wild-type yeast strain, which is sensitive to fructose and has a genotype of MATa HAP1⁺ can1Δ::TDH3ₚᵣ-yrKHK.

The gold standard broth microdilution test was performed by preparing the antibiotic solutions at their final concentrations from a stock solution of each examined antibiotic. A fresh 200 μL volume of each antibiotic solution (prepared in MEI culture medium) was pipetted into each microwell of a 96 MicroWell plate. Note, the concentrations of each antibiotic solution was set at 0.1-1 μg/mL (by 0.1 μg/mL-unit increment between every two consecutive antibiotic concentrations), 1-10 μg/mL (by 1 μg/mL-unit increment between every two consecutive antibiotic concentrations) and 10-100 μg/mL (by 10 μg/mL-unit increment between every two consecutive antibiotic concentrations). Then, 10 μL of each bacterial stock suspension was added to each microwell containing antibiotic solution to reach the appropriate bacterial final concentration (1×10⁶ CFU/mL). The bacteria was incubated in the presence of antibiotics for 20 h, and then the MIC and antimicrobial resistant assay time upon 80% reduction in the OD₆₀₀-growth curves was measured and compared to the negative control (i.e., without adding any antibiotic to the bacterial suspension). Bacteria were grown at 37° C. shaking for 24 h. Standardized growth curve analysis was performed using a Bioscreen C automated plate reader (Growth Curves USA, NJ) by measuring OD₆₀₀. All experiments were completed in triplicate and performed twice.

Computational fluid dynamics (CFD) simulations. COMSOL Multiphysics (version 5.3; COMSOL Inc., USA) was used to carry out to obtain the flow velocities and concentration profiles. 2D creeping flow module was used as the flow and boundary condition were considered as: (i) boundary condition: pressure 0 Pa; (ii) wall condition: no slip; (iii) considering the suppression of backflow; and (iv) normal physics-controlled for mesh size. The Navier-Stokes (Eq. 1) and conservation of mass (Eq. 2) equations, in which V denotes the velocity field, ρ is the density of the culture medium, P is pressure, and μ is the dynamic viscosity were solved.

ρ(V·∇V)=−∇P+∇·μ(∇V+(∇V)^{T} (1)

∇·V=0 (2)

Small-molecule diffusion coefficient determination. Based on the spectrophotometry method, 4 mL culture medium was loaded into a cuvette, followed by 80 μL of antibiotics (e.g., nalidixic acid, ampicillin, and cefuroxime plus resazurin as a fluorescent chemical; initial concentration=10 mg/mL) that was gently loaded at the bottom of the cuvette using a chromatography syringe. The diffusion kinetics of the antibiotics were obtained from the UV absorbance of their maximum wavelengths (correlated with concentration) as they diffused up through the cuvette over time.

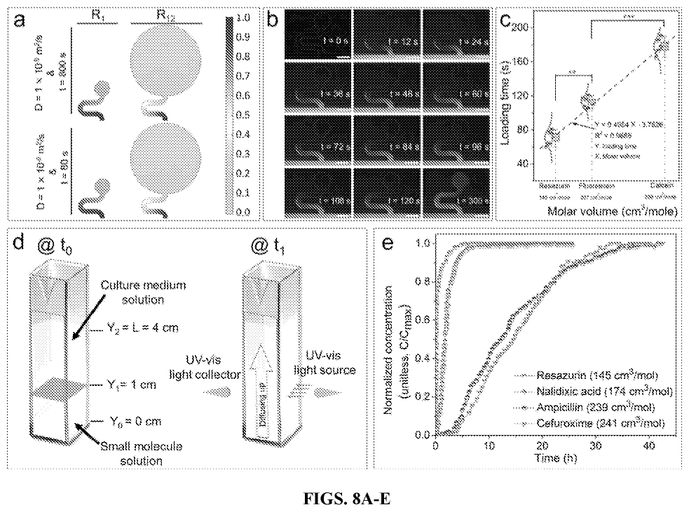

‘D₁×t₁=D₂×t₂’ derivation. To empirically determine the diffusion coefficients of the antibiotics in culture media, the UV absorbance spectrum of the antibiotics diffusing up in a cuvette was measured. These values (i.e., UV absorbance data-points) can be correlated with a mathematical equation. To obtain the theoretical model of mass transport in the cuvette, the mass transfer was considered in a Cartesian geometry due to the rectangular cuboid shape of the cuvette FIG. 8D. To simplify the equation, the mass transport was assumed to be one-dimensional (‘y’ direction: the cuvette height direction). Moreover, the antibiotic diffusion into the culture medium was considered as an unsteady-state phenomenon, as represented in Eq. 3, in which, C, t, and D denote the antibiotic concentration, time, and diffusion coefficient, respectively.

$$\frac{\partial C ( y , t )}{\partial t} = D \frac{{\partial }^{2} C ( y , t )}{\partial {y}^{2}} \qquad ( 3 )$$

By substituting t×D with as a variable (and tD=t′, consequently) into the Eq. 3, it can be simplified to Eq. 4:

$$\frac{\partial C ( y , {t}^{′} )}{\partial {t}^{′}} = \frac{{\partial }^{2} C ( y , {t}^{′} )}{\partial {y}^{2}} \qquad ( 4 )$$

Two insulated boundary conditions were considered in the ‘y’ direction (the top and bottom layers of solution in the cuvette). The boundary conditions and the initial conditions were considered as follows (Eq. 5 & 6):

$$\frac{\partial C ( 0 , t )}{\partial t} = 0 ⟹ \frac{\partial C ( 0 , {t}^{′} )}{\partial t} = 0 \qquad ( 5 )$$

$$\frac{\partial C ( L , t )}{\partial t} = 0 ⟹ \frac{\partial C ( L , t )}{\partial t} = 0 \qquad ( 6 )$$

Two first terms of the Taylor series were used as initial conditions, as follows:

$$exp ( - x 2 ) = 1 - {x}^{2} + \frac{{x}^{2}}{4} - \frac{{x}^{6}}{6} + \frac{{x}^{8}}{24} + … \qquad ( 7 )$$

Using the “Separation of Variables” method, the answer for Eq. 8 can be defined as follows:

C(y,t′)=Y(y)·T(t) (8)

Plugging Eq. 8 into Eq. 4 leads to the following equation:

$${T}^{′} \cdot Y = {Y}^{″} \cdot T ⟹ \frac{{T}^{′}}{T} = \frac{{Y}^{″}}{Y} = λ = - {k}^{2} = constant \qquad ( 9 )$$

Eq. 9 can be solved and simplified by using the boundary conditions.

$$\frac{C}{{C}_{0}} ( Y , {t}^{′} ) = {\sum }_{n = 0}^{∞} {a}_{n} \cdot {exp}^{\frac{- {n}^{2} {π}^{2}}{{L}^{2}} {t}^{′}} cos ( \frac{n π}{L} Y ) \qquad ( 10 )$$

Substituting tD=t′ into Eq. 10 leads to:

$$\frac{C}{{C}_{0}} ( y , t ) = {\sum }_{n = 0}^{∞} {a}_{n} \cdot {exp}^{\frac{- {n}^{2} {π}^{2}}{{L}^{2}} Dt} \cdot cos ( \frac{n π}{L} y ) \qquad ( 11 )$$

Using the initial condition to find a n results in Eq. 12:

$$\frac{C}{{C}_{0}} ( y , t ) = \frac{4}{3} + \frac{2 {L}^{3}}{{π}^{3}} {\sum }_{n = 1}^{∞} \frac{( - 2 ) {( - 1 )}^{n + 1}}{{n}^{2}} \cdot {exp}^{\frac{- {n}^{2} {π}^{2}}{{L}^{2}} Dt} \cdot cos ( \frac{n π}{L} y ) \qquad ( 12 )$$

For n>>2, the terms of series were negligible. Then, the two first sentences of Eq. 12 were used, which led to Eq. 11. The empirical data obtained with UV-Vis spectrophotometry was correlated with Eq. 11 and the diffusion coefficient for each antibiotic was then calculated:

$$\frac{C}{{C}_{0}} ( y , t ) = \frac{4}{3} + \frac{2 {L}^{3}}{{π}^{3}} ( 2 \cdot {exp}^{\frac{- {π}^{2}}{{L}^{2}} Dt} \cdot cos ( \frac{π}{L} ) y - \frac{1}{2} {exp}^{\frac{- 4 {π}^{2}}{{L}^{2}} Dt} \cdot cos ( \frac{2 π}{L} ) y ) \qquad ( 13 )$$

Imaging and Data Analysis. A ZOE™ fluorescent cell imager (Bio-Rad, CA) was used as an imaging platform to take the images of the microchambers, and ImageJ software was utilized to convert the fluorescent intensity to gray value. The fluorescence of the medium within the wells was calculated by averaging the pixel intensities in a given semi-spherical region. A custom MATLAB script was used to analyze the images.

### Example 2—MVM² Device Compartments and Operational Protocol

The MVM² platform (also referred to herein as a device) features four main-channels in parallel, with openings at the ends of each main-channel (i.e., A₁-A₄ and B₁-B₄; FIG. 6A). Moreover, arrays of dead-end microchambers, each containing a different volume increasing in size from R₁ to R₁₂, are connected to each side of the main-channels via identical serpentine side-channels (FIG. 6A and FIGS. 10A-D). FIG. 6B shows how the biological assay design is integrated with the MVM² platform, in which the microchambers connected to one of the main channels is reserved for the ‘negative control’ test (i.e., no small-molecule exposure), while the remaining three channels provide the positive low, medium, and high concentration ranges of the small-molecule at three orders of magnitude (e.g., 0.1-1, 1-10, and 10-10011 g/mL for the low, medium, and high ranges, respectively).

To perform a small-molecule-based biological assay in the MVM² platform, four main steps to load the reagents are followed. Step-i involves loading a suspension of the biological species (often with a fluorescent chemical indicator) into the MVM² platform so that it is uniformly distributed throughout (see FIG. 6C(i) and FIGS. 11A-D for more details; this step mainly takes ˜3-5 min). In step-ii, small-molecule solutions at C0, 0.1 C0, and 0.01 C0 concentrations are loaded into the high, medium, and low positive main-channels, respectively, which diffuse into the corresponding microchambers through the connected side-channels (see FIG. 6C(ii) and FIGS. 12A-D for more details; this step mainly takes as equal as the small-molecule loading time). To obtain a controllable and rational small-molecule loading time, the serpentine-like side-channels were designed (FIGS. 13A-B).

Biological suspension loading protocol and steps are shown in FIGS. 11A-D. The biological species suspension was initially loaded into the main-channels using opening A₁, while all openings B's were temporarily blocked using medical tape or tubing that could be closed (FIG. 11A). This allowed the bacterial suspension to flow through the main negative and positive channels and flush out from openings A₂-A₄ (only 4 circuits are shown as an example in FIGS. 11A-B for brevity). Upon filling the main-channels with the biological suspension, openings A₂-A₄ were then temporarily blocked using medical tape as well (FIG. 11B). Blocking openings A₂-A₄ and B₁-B₄ helped to continuously and gently push and flow the biological suspension into the microchambers through the side-channels, which can happen via the escape of entrapped air in the microchambers through the high fractional free volume of the PDMS walls (FIG. 11C) (see Chang et al. “Free Volume and Alcohol Transport Properties of PDMS Membranes: Insights of Nano-Structure and Interfacial Affinity from Molecular Modeling,” Journal of Membrane Science 417:119-130 (2012); Liu et al, “Point-of-Care Testing Based on Smartphone: The Current State-of-the-Art (2017-2018),” Biosensors and Bioelectronics 132:17-37 (2019); and Wu et al. “Enhancing the Interfacial Stability and Solvent-Resistant Property Of PDMS/PES Composite Membrane by Introducing A Bifunctional Aminosilane,” Journal of Membrane Science 337:61-69 (2009); each of which is hereby incorporated by reference in its entirety).

A resazurin red fluorescent solution (representing a biological sample) was used to experimentally illustrate the sample loading in microchamber R₁, which features the smallest volume. As shown in FIG. 11D, the solution completely fills R₁ within ˜36 s. Meanwhile, the largest microchamber (R₁₂, not shown) takes ˜3-4 min to fill. After successful loading of the biological suspension into the microchambers, all openings A₁-A₄ and B₁-B₄, which had been temporarily blocked during the loading process, are then opened and ready for the next step of loading the small-molecule solution (shown in FIG. 6C(ii)).

The protocol for small-molecule solution loading is shown in FIGS. 12A-D, which illustrates only four microfluidic circuits for brevity. For loading the small-molecule solution into the microchambers in order to observe the resulting gradient-based small-molecule concentration profile in the MVM² platform, a step-by-step protocol was followed as represented in FIGS. 12A-D. At the first step, openings A₂-A₄ and B₂-B₄ are blocked and biocompatible oil, HFE-7500 (shown with yellow color) is flowed into the negative main channel from port A l and flush out from outlet B₁ (FIG. 12A(ii)). This helps to isolate the microchambers connected to the negative main channel, which features the negative control microchambers (i.e., microchambers without any small-molecule loading).

To produce the low-range gradient-based concentration profile (GCP) of the small-molecule solution, openings A₁, A₃, A₄, B₁, B₃, and B₄ are temporarily blocked and the small-molecule solution is flowed into the main low-range positive channel (FIG. 12B(i)). Upon the diffusion of the small-molecule solution into the serpentine side-channels within the loading time (shown with light green color), while openings A₂, B₁, and B₂ are opened, the excess small-molecule solution from the main low-range positive channel is flushed out using the biocompatible oil. This consequently isolates the microchambers connected to this main channel (FIG. 12B(ii)). The same procedure for the medium- and high-range main channels is followed to load the small-molecule with targeted ranges into microchambers connected to the medium- and high-range main channels, as shown in FIG. 12C and FIG. 12D, respectively.

Criteria for microchamber and side-channel geometry design are shown in FIGS. 13A-B. In terms of side-channel geometry, the length of side-channels is the most important parameter which can play the role for drug loading. Longer side-channel length causes longer loading time for drug to pass the side-channel (the purple color in FIG. 13A). If the side-channel length (L) was designed to be short, then the loading time would be short, and as a consequence the operator might not have good control of drug loading (due to the short period of time between loading drug solution into main-channels and subsequently washing them with oil). For example, if the drug solution loading time was only 5 s (meaning the time-point that drug solution is loaded into main-channel until it was flushed with oil), a 1 second error by the operator in initiating the oil washing by 1 second sooner or later (i.e., at 4 s or 6 s) could incur a 20% error (1 s/5 s=20% error) in drug solution loading. In contrast, a drug loading time around 60 s or even longer can give an operator enough freedom to load the drug solution and accurately initiate the subsequent oil washing. It can also reduce the minimum error in terms of drug loading (for example, 1 second in 60 s of drug loading can cause only 1.6% error). Therefore, the side-channels' length was designed to obtain this order of loading time (FIG. 13A). Another important point regarding the side-channel geometry is about designing a compact microfluidic device and avoiding any wasted space. Therefore, a compact serpentine-structure of side-channel was used rather than a straight channel (FIG. 13B).

Importantly, there are two potential scenarios to make a gradient-based concentration profile (GCP) of the small-molecules in a platform: (a) exposing different amounts of a small-molecule with the same number of a biological species, or (b) exposing the same amount of a small-molecule with different numbers of a biological species. The second method was chosen in the MVM² platform as it requires exposing the same amounts of a small-molecule (i.e., identical green-color patterns in microchambers of the low, medium, or high range as shown in FIG. 6C(iii)) with different number of biological species, as loaded into multi-volume microchambers R₁-R₁₂ (note that the volume of R₁₂ is 10-times larger than R₁). This results in distinct C₁-C₁₂ concentrations within each low, medium, and high ranges (FIG. 6C(iv)).

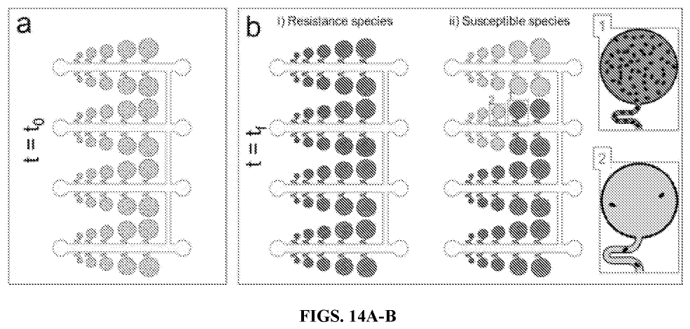

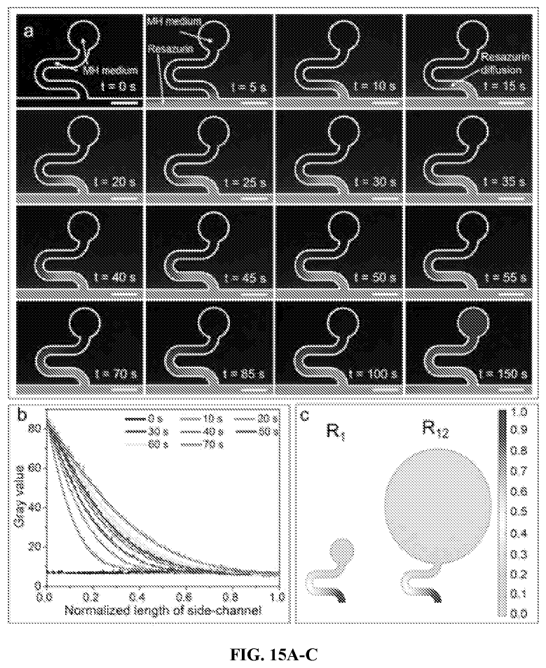

Such a GCP is achieved in FIG. 6C step-iii of the loading process by subsequently washing the main-channels with a biocompatible oil to stop further small-molecule loading and isolate the microchambers avoiding any chemical exchange between adjacent microchambers (taking ˜5-10 s). In step-iv (FIG. 6C(iv)), these small molecules are allowed to uniformly diffuse and distribute within each isolated microchamber, producing a GCP in each microchamber array. This MVM² design helps the investigation of the effects of dozens of small-molecule concentrations on a biological species, enabling important biological effects to be rapidly pinpointed in a single test (e.g., susceptibility or resistance of a biological species to a small-molecule drug, as schematically shown in FIGS. 14A-B).

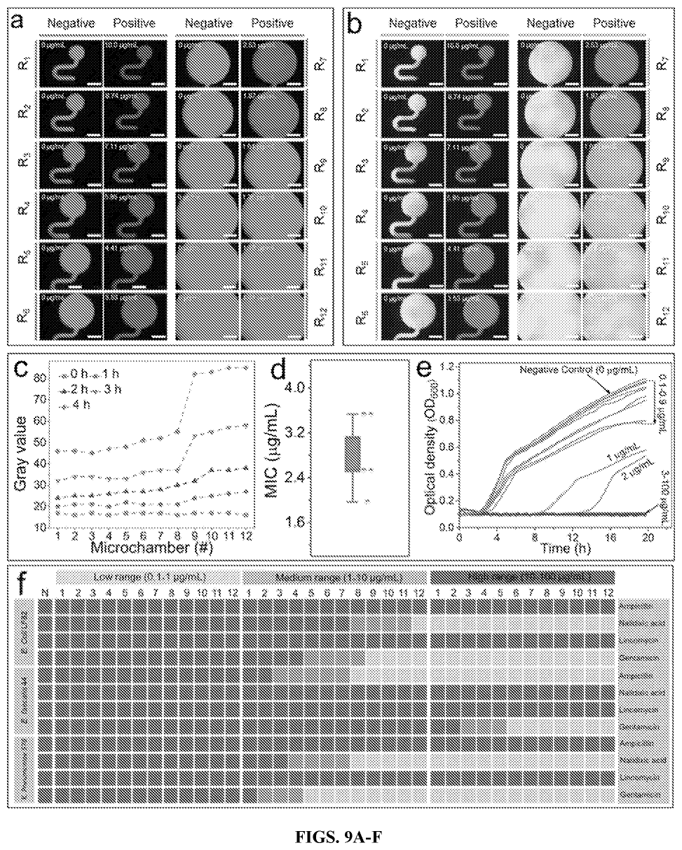

Studying two highly potential outcomes in biological small-molecule assay using MVM² platform is shown in FIGS. 14A-B. In contrast to the small-molecule GCP in the microchambers, the fluorescence intensities of the chemical indicator in all microchambers are the same at time zero (i.e., to) of a biological small-molecule assay, as shown by pink color in FIG. 14A). Here, two potential outcomes are discussed among others, as shown in FIG. 14B(i) and FIG. 14B(ii). If the small-molecule is not effective on a tested biological species, then the biological species grow in numbers. This causes the fluorescence intensities of the chemical indicator for all microchambers to increase, leading to no observable difference between the negative and positive microchambers (FIG. 14B(i)). However, if the tested small-molecule is effective at inhibiting or stopping the biological species growth (e.g., for bacteria susceptible to antibiotics), then the fluorescent intensities of the microchambers representing the negative controls (not exposed to the small-molecule) will continuously increase, while the fluorescence intensities of the positive microchambers will be different depending on the minimum inhibitory concentration (MIC) of the small-molecule (FIG. 9B(ii)). Below the MIC, the biological species can still survive, leading to higher fluorescence intensities (shown by microchamber 1 in FIG. 14B); while above the MIC, the biological species growth is stopped (shown by microchamber 2 in FIG. 14B) and no changes in the fluorescent intensities will be recorded (FIG. 14B(ii)).

### Example 3—MVM² Platform Characterization for a Typical Small-Molecule

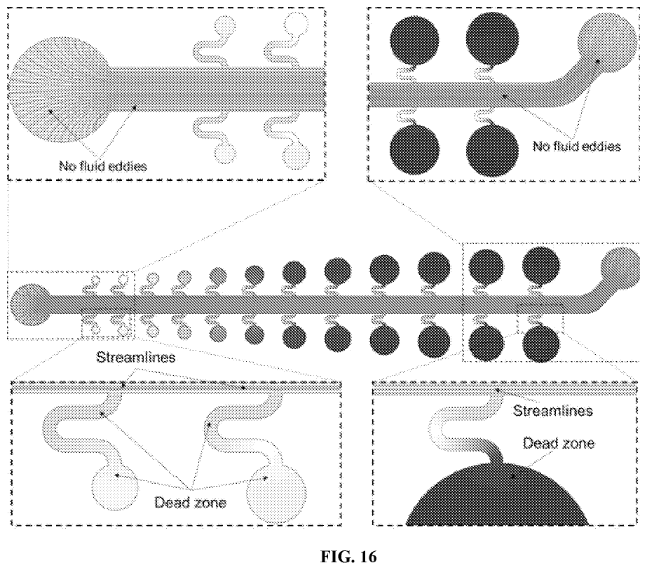

To validate the hypothesis of small-molecule loading into the multi-volume microchambers via diffusion in the MVM² platform, a 20% wt/v solution of resazurin (a fluorescent small-molecule) was used. The resazurin diffusion was monitored through a preloaded aqueous phase (Mueller-Hinton culture medium) and it was found that resazurin successfully moved through the side-channel and entered the microchamber (FIGS. 15A-C). Computational fluid dynamics (CFD) simulation of this process also demonstrated the formation of dead-zones in the microchambers and side-channels (FIG. 16). This indicates that the small molecules are driven into the microchambers by a diffusion mechanism alone (i.e., no mass transport through convention).

Small-molecules' self-diffusion is the mechanism for small-molecule loading into microchambers in MVM² platform as shown in FIG. 16. To ensure that small-molecule solution loading into side-channels and corresponding microchambers is happened via small-molecule diffusion mechanism (i.e., there is fluid dead-zones in the side-channel and corresponding connected microchamber), a CFD simulations approach was used. FIG. 16 shows the simulation and experimental results of dead-zone formation in the side-channel and corresponding microchamber, respectively.

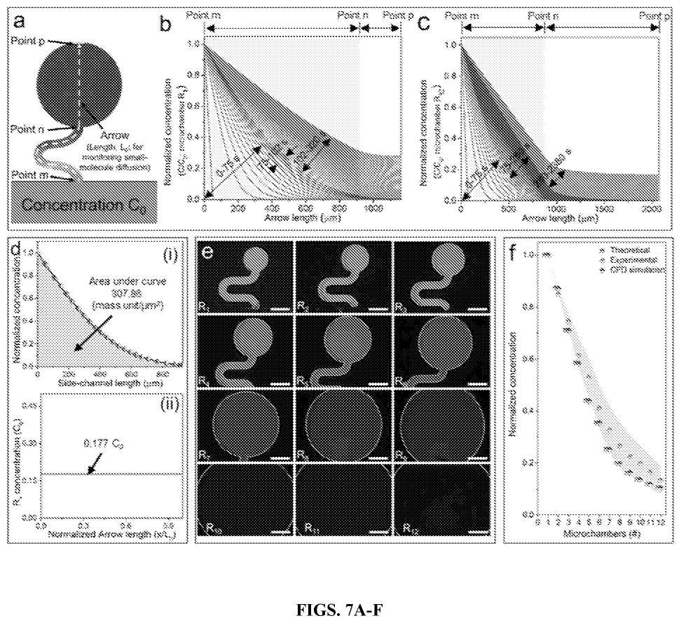

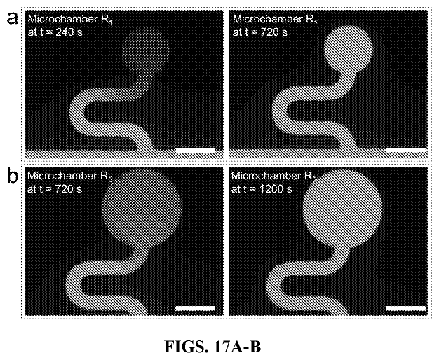

Control over the small-molecule diffusion into the microchambers is pivotal for obtaining a GCP, which is governed by the loading time (defined as the time period between loading the small molecule into the main channels and flushing the system with oil). For an unlimited loading time (co), the microchambers become fully saturated, leading to the failure of GCP formation (FIGS. 17A-B). Therefore, to find the optimum loading time for resazurin, its diffusion was monitored by measuring the position of the fluorescent solution along the side-channel and microchamber, as schematically shown by the white dashed arrow marked at point ‘m,’ ‘n,’ and ‘p’ in FIG. 7A.

Small-molecule gradient-based contrition failure within a long (unlimited) loading time in MVM² platform is shown in FIGS. 17A-B. In FIGS. 17A-B, it is shown that if there is not a robust control on small-molecule diffusion and loading by controlling the loading time, the small-molecule (a fluorescent dye in FIGS. 17A-B), can infinitely diffuse into all microchambers and saturate one by one over time (from the smallest microchamber to the largest one). This causes the failure of hypothesis for making a small-molecule GCP into MVM² platform, as shown for microchamber R₁ and R₅ in FIG. 17A and FIG. 17B, respectively. Loading the same amounts of a small-molecule into all microchambers R₁-R₁₂ in each row, as different numbers of biological species have been already loaded into all microchambers R₁-R₁₂ is the hypothesis of reproducing small-molecule GCP in MVM² platform.

The CFD-based time-dependent normalized concentration profiles—divided by the maximum concentration, C0—along the dashed arrow for microchambers R₁ (smallest) and R₁₂ (largest) were obtained (FIGS. 7B-C). As shown in FIG. 7B and FIG. 7C, the kinetics of resazurin loading follows three phases before the small-molecule fully saturates the aqueous medium in the microchambers (i.e., a normalized concentration of 1). In phase-i, the resazurin solution loaded into the main channel (normalized concentration of 1) moves along the side-channel—from point m to point n—within 75 s (pink curves in FIG. 7B and FIG. 7C). In phase-ii, more resazurin diffuses into the side-channels and microchambers, eventually reaching point p (blue curves in FIG. 7B and FIG. 7C). Finally, even more resazurin diffuses and the concentration of every position along the dashed arrow increases, culminating in saturation of the features (phase-iii; dark yellow curves in FIG. 7B and FIG. 7C).

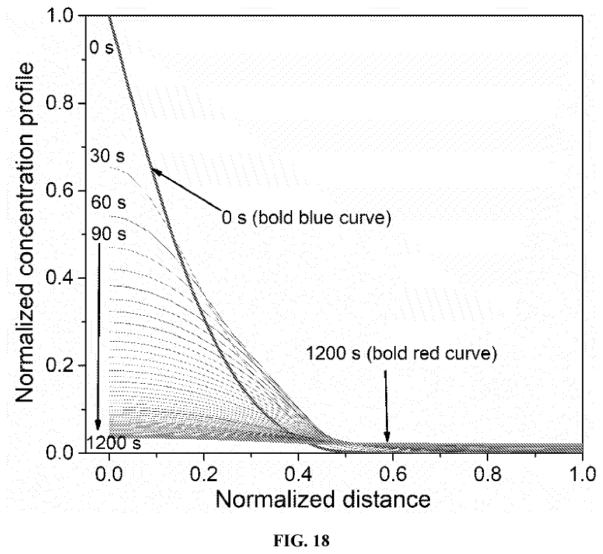

To investigate the criterion for achieving resazurin GCP in the MVM² platform (i.e., loading the same amount of resazurin into the multi-volume microchambers at a specific time-point), the concentration profile at t=75 s (pink curve, FIG. 7B and FIG. 7C) was chosen as only the first phase (diffusion through the side-channels) was the same for all microchambers R₁-R₁₂. The concentration profiles at t=75 s (after washing off with the biocompatible oil) and t>>75 s (shown in FIG. 7D(i) and FIG. 7D(ii), respectively) demonstrate the before and after of the uniform distribution of the small-molecule concentration in the side-channel and corresponding microchamber R₁. The area under the curve before uniform distribution was calculated (FIG. 7D(i)), which represents the net amount of resazurin (307.86 mass unit/μm2) loaded into just microchamber R1's side-channel. This would result in microchamber R₁ obtaining a uniform resazurin concentration profile of 0.177 C0 after the even distribution of resazurin into both the side-channel and its connected microchamber R₁ (FIG. 7D(ii)). Meanwhile microchamber R₁₂, which features 10-times larger volume than R₁, would feature a 10-times lower concentration (0.0177 C0), while the remaining multi-volume microchambers (R₂-R₁₁) achieve an intervening range of concentrations (0.177 C0<C<0.0177 C0), resulting in the successful formation of a GCP. Logically, there are different time periods for microchambers with different sizes (R₁-R₁₂) to obtain uniform small-molecule drug distribution. The largest microchamber (R₁₂) requires the longest time to have the small-molecule drug fully distributed into it. Therefore, CFD simulations for the microchamber R₁₂ were performed to find out how long it takes for a small-molecule such as resazurin to be uniformly distributed (FIG. 18).

As can be seen in FIG. 18, resazurin is fully distributed into microchamber R₁₂ below 1200 s (20 min). Note, this time-period is included as part of the assay incubation time (4 h) and the operator does not need to wait for drug distribution. For other small-molecules, the time depends on their molecular volume. However, this short period of drug uniform distribution is not significant compared to the total assay time of 4-5 h.

The time-point found by CFD simulations (t=75 s) was used to experimentally verify that resazurin can achieve a GCP in the MVM² device. After loading the resazurin into microchambers R₁-R₁₂ for 75 s (as the loading time), the fluorescence of the microchambers was found to decrease with increasing microchamber size, indicating a successful GCP (FIG. 7E). The volume of all microchambers is known. This helped to determine the concentrations of microchambers theoretically, if hypothetically the same amounts of small-molecules were loaded into microchambers R₁-R₁₂. Upon normalization of microchambers' concentrations using Eq. 14, the theoretical normalized concentrations of all microchambers were calculated to compare with the experimental ones (FIG. 7F).

$$Normalized {concentration}_{{microchamber}_{Ri}} = \frac{{Volume}_{{microchamber}_{R 1}}}{{Volume}_{microcha {mber}_{Ri}}} \qquad ( 14 )$$

FIG. 7F confirms good agreement between the normalized GCPs obtained by experimental and CFD simulation approaches with the theoretical GCP (obtained using Eq. 14).

### Example 4—Versatility of the MVM² Platform for Biological Small-Molecules

diffusion of small-molecules into the multi-volume microchambers is the key for producing a GCP. However, it is well-known that small-molecules have different diffusion coefficients, which is most impacted by their molecular size. As a result, loading small-molecules with different diffusion coefficients at the same loading time could result in different GCPs in the MVM² platform, as shown in FIG. 19A, FIG. 19C, FIG. 20, and FIGS. 21A-B.

The concentration profiles into twelve microchambers was obtained using CFD simulations for the diffusion coefficients 5×10⁻⁹ m²/s (FIG. 21A). The concentrations were normalized into twelve microchambers for six diffusion coefficients as it is shown that one order of magnitude targeted gradient-concentration profile fails upon loading small-molecules with higher diffusion coefficients such as 5×10⁻⁹ m²/s and 2×10⁻⁹ m²/s FIG. 21B).

Using an analytical solution for small-molecule mass transport in the side-channels and microchambers and CFD simulations for microchambers R₁ and R₁₂ (FIG. 8A and FIG. 22), it was found that the loading time (t) and diffusion coefficients (D) for any two small-molecules follow the relationship D₁×t₁=D₂×t₂. This relationship can help determine the loading time (e.g., t₂) for achieving a GCP for any biological small-molecule based on the loading time and diffusion coefficient of a known small-molecule (e.g., resazurin with t₁=75 s and

$${D}_{1} = 1.06 \times 1 {0}^{- 6} \frac{{cm}^{2}}{s} )$$

as well as the diffusion coefficient of the target small-molecule (e.g., D₂).

CFD simulations to confirm the relationship between small-molecule diffusion coefficients and loading times are shown in FIG. 22. It was qualitatively shown that the relationship D₁×t₁=D₂×t₂ between small-molecule diffusion coefficients and the loading is obeyed. In FIG. 22, it was quantitatively shown that exactly the same concentration profiles are obtained for two different small-molecules following the relationship D₁×t₁=D₂×t₂.

However, there is no extensive database available for the diffusion coefficients of biological small-molecules. Therefore, instead the small molecule's molar volume was chosen to investigate its relationship with the loading time, as the molar volume is more readily accessible compared to the diffusion coefficient. Toward this aim, the loading kinetics of different fluorescent dyes, including calcein (FIG. 8B), fluorescein (FIG. 23), and resazurin (FIG. 15A) were studied in the R₁ side-channel and microchamber. Note, the molar volumes of calcein, fluorescein, and resazurin are 356±5 cm³/mole, 208±4, and 145±7, respectively.

The kinetic for fluorescein dye diffusion into microchamber R₁ is studied in FIG. 23 and the outcomes were compared with resazurin and calcein fluorescent dyes' loading time (FIG. 8C) to correlate the molar volume of small molecules with the diffusion coefficients and consequently loading time in MVM² platform.

Interrogating the loading kinetics of the fluorescent dyes into the side-channel helped to establish a linear relationship between the small-molecules' molar volumes and loading times (R²=0.9885) in the MVM² platform, as follows (FIG. 19D and FIG. 8C).

Loading time=0.4954×molar volume−3.7623 Eq. 15

To further confirm that Eq. 15 can also apply to non-fluorescent biological small-molecules, a simple method using spectrophotometry was employed. By gently loading a biological small-molecule solution at the bottom of a cuvette preloaded with non-selective Mueller-Hinton culture medium, the maximum absorbance wavelength of the small-molecule was able to be recorded as it diffused up in solution along the cuvette height (FIG. 8D and FIG. 24).

Finding the small-molecule diffusion coefficients and diffusion time is shown in FIG. 24. For demonstration of small molecules' diffusion into a cuvette for measuring the diffusion coefficients, an experiment was performed using a food-grade dye loading at the bottom of a cuvette and the dye diffusion was monitored over a 32-h time-lapse experiment, as it diffused up and caused a homogenous distribution of blue dye into cuvette (FIG. 24).

This experiment allowed an easy correlation between the small molecule's diffusion and its absorbance as it moved upward along the cuvette (mimicking the small molecule diffusion along the side-channels). To test the validity of this methodology for determining the mass transport of small-molecules, three antibiotics were chosen as biological small-molecules (nalidixic acid, cefuroxime, and ampicillin) in addition to resazurin. The results indicated that the small-molecules with similar molar volumes (e.g., resazurin/nalidixic acid and cefuroxime/ampicillin) show very close absorbance curves over time (i.e., similar diffusion patterns in the cuvette solution; FIG. 8E). This result confirms that the easily-found molar volume of a small-molecule can be used to calculate the appropriate loading time in the microfluidic device based on Equation-1, rather than relying on the more difficult-to-determine diffusion coefficient. Based on this methodology, the loading times of most commercial antibiotics, antifungal, and anticancer drugs are provided in Tables 1-3, allowing this platform to be employed for further studies.

### Example 5—MVM² Platform Testing for Eukaryotic and Prokaryotic Cells

In terms of practical application of the MVM² platform for different biological assays, water evaporation can become an issue for nanoliter-sized culture media in the microchambers due to the permeability of the PDMS walls (FIGS. 25A-C). This can lead to small-molecule concentration changes, particularly for long-term biological assay measurements. To overcome this issue, a water bath design has been added to the MVM² device platform to obtain evaporative equilibrium (FIG. 26A-D). The simple design improves the device's capability to run long-term biological assays, such as cancer cell-anticancer drug testing (e.g., >24 h), as well as relatively short-term ones, such as bacteria-antibiotic testing (e.g., <8 h; FIG. 27).

Techniques to minimize water evaporation from the MVM² platform for long-term biological suspension cultures are shown in FIGS. 25A-C, FIGS. 26A-D, and FIG. 27. In detail, after loading the bacterial suspension, drug solution, and washing the main channel with oil to isolate the microchambers, nano-liter volumes of culture medium have been loaded in microchambers with different sizes. Water as the main part of culture medium has the chance to evaporate through pores of the PDMS ceiling and side-walls, as shown in FIGS. 25A-C. Therefore, two water baths are employed to equilibrate water evaporation through both the ceiling and side-wall. The first water bath has been designed in the PDMS device, as it makes an equilibrium between water escape from microchambers' side-wall (FIG. 26A-C). The second bath is created by immersing the device into container such as petri-dish filled with water to make an evaporation equilibrium within the ceiling and outside environment (FIG. 26D). After using the water bath technique for minimizing water evaporation, water evaporation was effectively avoided for a long-term experiment (as typically studied for a 17 h experiment as shown for microchamber R₁ in FIG. 27). It is noteworthy to mention that this time is enough for most of biological screening experiments, although it is feasible to run the experiment for longer period without any issue or concern about water evaporation, as well.

To further examine the functionality of the MVM² microfluidic device and due to the importance of worldwide emerging resistance to antibacterial drugs, the susceptibility of a green fluorescent protein (GFP)-labeled E. coli 541-15 to gentamicin (a typical antibiotic) was studied in order to determine the minimum inhibitory concentration (MIC) of an antibiotic. The E. coli 541-15 bacterial suspension (concentration: 1×10⁶ CFU/mL) was first loaded into the microchambers (step i). Resazurin (5 wt %) was also added to the bacterial suspension to allow monitoring of the bacterial cell metabolism through an irreversible resazurin-resorufin enzymatic reduction reaction (i.e., with increasing bacterial growth, the higher resazurin reduction results in greater fluorescent intensity). A GCP of the gentamicin (loading time=170 s; Table 1) was generated in the positive microchambers (step-ii) in the concentration range of 0.1-100 μg/mL, specifically 0.1-1 (low), 1-10 (medium), and 10-100 (high) μg/mL for the three channels. At t=0 h, the negative and positive microchambers feature the same low red fluorescent intensities of resazurin, as expected (FIG. 28). After incubation of the bacteria for 4 h at 37° C., the red fluorescent intensities of all the negative controls (i.e., no gentamicin exposure) increased, indicating bacterial growth (FIG. 9A). Additionally, fluorescent intensities as high as the negative controls were obtained for positive microchambers R₉-R₁₂, suggesting the antibiotic concentration in these microchambers was not sufficient to retard/stop the bacterial growth. Meanwhile, the other positive microchambers gentamicin concentration C₁-C₈>C₉-C₁₂) showed relatively lower fluorescent intensity. These results clearly indicated the MIC at which the E. coli 541-15 was susceptible to the gentamicin.

To further confirm these findings based on the bacterial cell metabolism obtained from resazurin reduction, the red fluorescent intensities were correlated with the green fluorescence directly associated with the GFP-labeled bacterial growth in the corresponding microchambers (FIG. 9A and FIG. 9B). Both red and green fluorescent modes confirmed excellent correlation between the bacterial growth and resazurin reduction. By converting the red fluorescent intensities to gray values, the MIC of gentamicin was calculated as 2.82±0.68 μg/mL (n=5) (FIG. 9C and FIG. 9D). Moreover, the gold standard broth microdilution technique (measuring the bacterial cell density—OD₆₀₀—vs. the incubation time) was used to validate the MVM² microfluidic device functionality (FIG. 9E). The MIC for the E. coli 541-15/gentamicin pair was obtained as 3±2 μg/mL (n=5), which is in excellent agreement with the MVM² finding of 2.82±0.68 μg/mL (n=5).

As a representative small molecule model (assay), showing a bacterial resistance to antibiotics, the ampicillin-E. coli 541-15 pair assay was also examined. Similarly, bacterial cell metabolism and growth using the resazurin reduction assay was probed and changes in the number of GFP-labeled bacteria during the assay was monitored, as shown in FIGS. 29A-B and FIGS. 30A-B, respectively. There was no significant difference in the red or green fluorescent intensities of the negative control (0 μg/mL) and high range (10-100 μg/mL), confirming the bacterial resistance to ampicillin. Note, the data for the low and medium ranges are not shown in FIGS. 29A-B and FIGS. 30A-B, respectively. Moreover, the broth microdilution assay was also used to confirm this finding for the E. coli 541-15-ampicillin pair in the MVM² platform, which also showed the resistance of E. coli 541-15 to ampicillin (FIG. 31).

To further evaluate the platform functionality, isolated bacteria from two clinical scenarios were tested: ((i) ileal mucosa of human patients associated with Crohn's disease in FIG. 9F and (ii) bovine mastitis in FIG. 32 vs. relevant antibiotics, testing the most important mechanisms of action for antibiotics. FIG. 9F and FIG. 32 contain the following information: (i) the type of bacteria and tested antibiotic can be found at the left and right sides, respectively; (ii) each row includes 37 squares, as labeled at the top and have been categorized in 4 different categories—first square representing the microchamber as negative control (labeled with “N”), and every next twelve squares as respectively representing low, medium, and high positive ranges of antibiotic concentrations; (iii) the low, medium, and high ranges are included the concentration ranges of 0.1-1, 1-10, and 10-100 μg/mL; and (iv) there are three types of squares: dark red, dark pink or light pink. If bacteria is susceptible to a specific antibiotic (e.g., E. coli LF82/gentamicin pair in FIG. 9F), below MIC (shown by dark pink color), bacteria can survive and grow causing more resazurin reduction (shown by dark red). At concentrations greater than the MIC, the bacterial growth stopped as shown by light pink color. The dark pink squares also show the microchambers which antimicrobial susceptibility was monitored at different trials. As bacteria resistance to antibiotic (e.g., E. coli LF82/ampicillin pair in FIG. 9F), then all microchambers are dark red demonstrating the well-grown bacteria in the presence of the tested antibiotic in microchambers. For ileal mucosa of human patients associated with Crohn's disease (FIG. 9F), the most effective antibiotic was gentamicin, effective through inhibiting protein synthesis by targeting 30S subunit of ribosome.

Clinical models for testing the MVM² microfluidic device are shown in FIG. 32. Bovine with mastitis disease was also used to test the MVM² platform for deciphering their antimicrobial resistant/susceptibility profile. These antibiotics were chosen to investigate the most common and important antibiotics' mechanisms of action, including the disruption of bacterial cell wall and membrane synthesis, binding to RNA/DNA and interrupting nucleic acid replication, and inhibition of protein synthesis by binding to ribosome. FIG. 32 contains the following information: (i) the type of bacteria and tested antibiotic can be found at the left and right sides, respectively; (ii) each row includes 37 squares, as labeled at the top and have been categorized in 4 different categories—first square representing the microchamber as negative control (labeled with “N”), and every next twelve squares as respectively representing low, medium, and high positive ranges of antibiotic concentrations; (iii) the low, medium, and high ranges are included the concentration ranges of 0.1-1, 1-10, and 10-100 μg/mL; (iv) there are three types of squares: dark red, dark pink or light pink. If bacteria is susceptible to a specific antibiotic (e.g., E. coli/cefuroxime pair, see FIG. 32), below MIC (shown by dark pink color), bacteria can survive and grow causing more resazurin reduction (shown by dark red). At concentrations greater than the MIC, the bacterial growth stopped as shown by light pink color. The dark pink squares also show the microchambers which antimicrobial susceptibility was monitored at different trials. As bacteria resistance to antibiotic (e.g., E. coli LF82/lincomycin pair, shown in FIG. 32), then all microchambers are dark red demonstrating the well-grown bacteria in the presence of the tested antibiotic in microchambers. For bovine mastitis and as shown in FIG. 32, the most effective antibiotics were cefuroxime, kanamycin/gentamicin, and nalidixic acid, which are disrupting cell wall/membrane synthesis, inhibiting protein synthesis by targeting 30S subunit of ribosome, and deactivation of DNA gryase, respectively. While, lincomycin, the only tested antibiotic inhibiting protein synthesis via binding to 50S subunit of ribosome, was not effective to kill or stop bacterial growth.

To probe the functionality of our MVM² platform for eukaryotic cells, its long-term cell-culture and growth capability was shown for cancer and yeast cells (the MCF-7 human breast cancer cell line and a Saccharomyces cerevisiae strain, respectively) in FIG. 33 and FIGS. 34A-B.

Loading cancer cell line into MVM² microfluidic device is shown in FIG. 33. One of the main interesting area of biological assays is testing small molecules effective in cancer cell biology. To do this, it is important to show that cancer cells can survive in the MVM² platform through studying (i) the capabilities of cancer cell adhesion (attachment) to the PDMS-based substrate, (ii) the assurance of having enough nutrient loaded into microchambers during the study, and (iii) the capabilities of CO₂ transport through PDMS wall for a relatively long-term biological assay. In this case, a breast cancer cell line (MCF-7) was utilized for loading into MVM² platform and probing the capabilities of PDMS-made MVM² platform for providing an appropriate substrate for cell adhesion and growth. This experiment was performed for 3 days (72 h) showing that the initial culture medium loaded into microchambers was enough to feed the seeded cancer cells. Moreover, it was illustrated that there is appropriate CO₂ transport though the PDMS cell walls, which is necessary for cell culture. As shown in FIG. 33, the cells are healthy (rounded-shape) upon loading into microchamber at t=0 h. The cells' adhesion was monitored and growth at t=24 h and t=48 h culture, as it shows excellent adhesions and growths (appropriate CO₂ transport and having enough nutrient) within these time periods. However, the cells started to metastasize and degrade as checked at t=72 h—i.e., the lack of enough nutrient can be the potential interpretation for cell metastasis.

Capabilities of yeast growth in MVM² device for a long-term run are shown in FIGS. 34A-B. The capabilities of MVM² platform for a long-term yeast growth was studied by loading a wild-type S. cerevisiae yeast strain into our MVM² platform and performing a 10 h culture. As can be seen from FIG. 34A, the wild-type S. cerevisiae cells could successfully grow and increased numbers of yeast cells can be observed in selected microchambers R₁ and R₁₀. Moreover, resazurin was used as a fluorescence indicator (applicable for showing healthy eukaryote and prokaryote cell metabolism) to confirm the yeast metabolism as the fluorescence intensity highly increased within the 10 h culture study (FIG. 34B).

Then, as an example, the platform was used to study recessive human metabolic diseases, specifically measuring sugar-phosphate toxicity. For these experiments, a strain of S. cerevisiae was chosen in which fructose, but not glucose, is toxic due to constitutive expression of a rat liver ketohexokinase gene (the yrKHK strain, DBY12549). The data obtained using the MVM² platform precisely pinpointed the binary ‘Yes/No’ sensitivity response of the yrKHK strain to fructose (Yes) and glucose (No), as shown in FIGS. 35A-C and FIGS. 36A-C, respectively.

In this case, the functionality and validity of the MVM² platform was used to test for these known “eukaryote cells/sugars” pairs. Consequently, sugars (fructose and glucose) were chosen as the small molecules to show the feasibility of testing small molecules on eukaryotic cells in the MVM² platform. A wild-type S. cerevisiae, DBY12000, yeast strain (namely, WT) and its genetically modified DBY12549 mutant (called yrKHK) were chosen.. The sensitivity/resistance of WT and yrKHK yeast strains has been reported using conventional biological approaches elsewhere. Briefly, the WT strain is both glucose- and fructose-resistant. But the genetically modified yrKHK strain is, however, fructose sensitive and glucose-resistant.

Sugars were tested in a wide-range of concentrations (0.007%-7%, three orders of magnitude). In this respect, low, medium, and high ranges of sugars featured 0.007-0.07, 0.07-0.7, and 0.7-7% sugar concentration ranges while the negative control-labeled microchambers were not exposed with the tested sugar. The yeast cell density was set at OD₆₀₀=0.1 (equals to 1×10⁶ cell/mL) for loading into microchambers and then expose with sugars and tested within the sugar concentration ranges of 0.007-7 wt %.

Moreover, a fructose concentration of 1.13 wt % was determined as the critical sensitivity concentration for the yrKHK strain using the MVM² platform (FIGS. 35A-C and FIGS. 36A-C), which is in excellent agreement with the analogous growth curves (FIG. 36C). Finally, the resistant outcome of a wild-type S. cerevisiae strain (DBY12000) to fructose and glucose was also studied, in which the wild-type S. cerevisiae growth continued regardless of the sugar concentrations as expected (FIGS. 37A-D and FIG. 38).

Wild-type vs. Glucose (outcome: not sensitive): In FIGS. 37A-D and FIG. 38, the WT yeast strain exposed with glucose and fructose, respectively. The yeast growth was monitored for any “yeast/sugar” pair within 10 h culture at two time points (t=0 and 10 h). Resazurin or any other chemical indicators were not used in these experiments, as the size of yeast is big enough to easily monitor the yeast growth through changing opacity or using a bright field microscopy.

TABLE 1
Antimicrobial Agents/Drugs, Loading Time
to Reproduce Gradient-Based Concentration

| Chemical, drug | Molar volume (cm³) | Loading time (s) |
|---|---|---|
| Actinomycin D | 880.6 ± 7.0 | 432.4 |
| Actinonin | 338.1 ± 3.0 | 163.7 |
| Aculeacin A | 754.1 ± 5.0 | 369.8 |
| Acycloguanosine (Aciclovir) | 127.1 ± 7.0 | 59.2 |
| Adenine 9-β-D-arabinofuranoside (Vidarabine) | 128.1 ± 7.0 | 59.6 |
| Alamethicin | 1583.4 ± 3.0 | 780.6 |
| L-Alanyl-L-1-aminoethylphosphonic acid | 140.5 ± 3.0 | 65.8 |
| (Alafosfalin) | | |
| Albendazole (Methyl 5-(propylthio)-2- | 203.1 ± 5.0 | 96.8 |
| benzimidazolecarbamat) | | |
| 17-(Allylamino)-17-demethoxygeldanamycin | 483.3 ± 5.0 | 235.6 |
| (Tanespimycin) | | |
| Amastatin | 386.4 ± 3.0 | 187.6 |
| Amikacin | 363.9 ± 5.0 | 176.5 |
| 7-Aminoactinomycin D (7-ADD) | 877.3 ± 7.0 | 430.8 |
| 7-Aminocephalosporanic acid (7-ACA) | 170.0 ± 5.0 | 80.4 |
| 7-Aminodesacetoxycephalosporanic acid (7- | 134.0 ± 5.0 | 62.6 |
| ADCA) | | |
| (+)-6-Aminopenicillanic acid (6-APA) | 142.0 ± 5.0 | 66.5 |
| Amoxicillin | 236.2 ± 5.0 | 113.2 |
| Amphotericin B (Fungizone) | 689.4 ± 5.0 | 337.7 |
| Ampicillin (D-(−)-α-Aminobenzylpenicillin) | 239.3 ± 5.0 | 114.7 |
| Anhydroerythromycin A | 590.7 ± 5.0 | 288.8 |
| (Erythromycin, or 2BR5PL6H3) | | |
| Anisomycin (or, Flagecidin) | 218.6 ± 5.0 | 104.5 |
| Aphidicolin | 275.9 ± 5.0 | 132.9 |
| Apicidin | 489.8 ± 7.0 | 238.8 |
| Apoptolidin (FU 40A) | 898.5 ± 5.0 | 441.3 |
| Apramycin (Nebramycin II) | 344.4 ± 5.0 | 166.8 |
| Artesunate | 292.2 ± 5.0 | 140.9 |
| Ascochlorin (Ilicicolin D, NSC 287492) | 337.6 ± 3.0 | 163.4 |
| Ascomycin (KD4185000) | 661.8 ± 5.0 | 324.1 |
| Azacitidine (Ladakamycin) | 117.0 ± 7.0 | 54.2 |
| Azithromycin | 632.6 ± 5.0 | 309.6 |
| Azlocillin (D-α-([Imidazolidin-2-on-1- | 296.6 ± 5.0 | 143.2 |
| yl]carbonylamino)benzylpenicillin) | | |
| Bacitracin | 994.4 ± 7.0 | 488.8 |
| Bafilomycin A1 (4730700) | 552.3 ± 5.0 | 269.8 |
| Bafilomycin B1 | 667.1 ± 5.0 | 326.7 |
| Bestatin (Ubenimex) | 257.5 ± 3.0 | 123.8 |
| Bithionol | 202.7 ± 5.0 | 96.6 |
| Blasticidine (Blasticidin S) | 261.9 ± 7.0 | 125.9 |
| Borrelidin | 426.5 ± 5.0 | 207.5 |
| Brefeldin A (Ascotoxin, BFA, Cyanein, | 252.9 ± 3.0 | 121.5 |
| Decumbin) | | |
| Caerulomycin A (Carulomycin A, Cerulomycin) | 185.8 ± 7.0 | 88.3 |
| Calcium ionophore III (ANTIBIOTIC A 23187, | 406.9 ± 5.0 | 197.8 |
| Calimycin) | | |
| (S)-(+)-Camptothecin | 230.2 ± 5.0 | 110.3 |
| (Camptothecin) | | |
| Carbenicillin (α-Carboxybenzylpenicillin) | 246.1 ± 5.0 | 118.2 |
| Cefaclor | 226.4 ± 5.0 | 108.4 |
| Cefalexin | 231.3 ± 5.0 | 110.8 |
| Cefazolin | 225.4 ± 7.0 | 107.9 |
| Cefixime | 244.4 ± 7.0 | 117.3 |
| Cefmetazole | 268.3 ± 7.0 | 129.2 |
| Cefoperazone | 363.6 ± 7.0 | 176.4 |
| Cefotaxime ((Z)-Cefotaxime) | 252.8 ± 7.0 | 121.5 |
| Cefmetazole | 268.3 ± 7.0 | 129.2 |
| Cefoperazone | 363.6 ± 7.0 | 176.3 |
| Cefsulodin (Ulfaret) | 316.5 ± 7.0 | 153.0 |
| Ceftazidime | 330.2 ± 7.0 | 159.8 |
| Ceftriaxone | 281.7 ± 7.0 | 135.8 |
| Cephalexin | 231.3 ± 5.0 | 110.8 |
| Cephalomannine (NSC 318735) | 609.9 ± 5.0 | 298.4 |
| Cephalothin (Cefalotin) | 252.7 ± 5.0 | 121.4 |
| Cephradine (Cefradine) | 237.2 ± 5.0 | 113.7 |
| Cercosporin | 335.5 ± 5.0 | 162.4 |
| Cerulenin | 196.7 ± 3.0 | 93.7 |
| Chloramphenicol | 208.8 ± 3.0 | 99.7 |
| Chlorhexidine | 363.3 ± 7.0 | 176.2 |
| Chloroquine | 287.8 ± 3.0 | 138.8 |
| Chlortetracycline | 281.0 ± 5.0 | 135.4 |
| Chromomycin A3 | 827.2 ± 5.0 | 406.0 |
| Chrysomycin A (MFCD07370133) | 361.7 ± 3.0 | 175.4 |
| Chrysomycin B (MFCD07370132) | 352.0 ± 3.0 | 170.6 |
| Cinoxacin | 159.7 ± 7.0 | 75.3 |
| Clarithromycin | 631.9 ± 5.0 | 309.3 |
| Clindamycin (Cleocin) | 327.2 ± 5.0 | 158.3 |
| Clofazimine | 366.0 ± 7.0 | 177.5 |
| Clotrimazole | 302.7 ± 7.0 | 146.2 |
| cloxacillin | 279.2 ± 5.0 | 134.5 |
| Colistin | 916.9 ± 5.0 | 450.4 |
| Concanamycin A (Folimycin) | 703.1 ± 7.0 | 344.5 |
| Cordycepin (3′-Deoxyadenosine) | 130.8 ± 7.0 | 61.0 |
| Coumermycin Al | 718.6 ± 5.0 | 352.2 |
| Cryptotanshinone (Tanshinone C) | 239.4 ± 5.0 | 114.8 |
| Cycloheximide (Actidione, Naramycin A) | 247.5 ± 3.0 | 118.8 |
| D-Cycloserine | 79.8 ± 3.0 | 35.8 |
| Cyclosporin A (Antibiotic S 7481F1, | 1183.6 ± 3.0 | 582.6 |
| Cyclosporine) | | |
| Cytochalasin D (Zygosporin A, 1632828) | 410.0 ± 5.0 | 199.3 |
| Cytochalasin B (Phomin) | 396.8 ± 5.0 | 192.8 |
| Dacarbazine ((E)-Dacarbazine) | 122.6 ± 7.0 | 56.9 |
| Daptomycin | 1110.7 ± 5.0 | 546.4 |
| Daunorubicin (Daunomycin) | 339.4 ± 5.0 | 164.3 |
| 10-Deacetylbaccatin III | 385.5 ± 5.0 | 187.2 |
| Demeclocycline | 265.1 ± 5.0 | 127.5 |
| 1-Deoxymannojirimycin | 112.0 ± 3.0 | 51.7 |
| Dichlorophene | 189.5 ± 3.0 | 90.1 |
| Dicloxacillin | 290.0 ± 5.0 | 139.9 |
| Difloxacin | 283.2 ± 3.0 | 136.5 |
| Dihydrostreptomycin | 293.4 ± 7.0 | 141.5 |
| Dimetridazole | 103.4 ± 7.0 | 47.4 |
| Dirithromycin | 700.6 ± 5.0 | 343.3 |
| Doxorubicin | 336.6 ± 5.0 | 162.9 |
| Doxycycline | 271.1 ± 5.0 | 130.5 |
| Duramycin | 1370.8 ± 5.0 | 675.3 |
| Econazole | 286.7 ± 7.0 | 138.3 |
| Embelin (Embelic acid, Emberine) | 260.1 ± 3.0 | 125.1 |
| Emetine | 407.9 ± 5.0 | 198.3 |
| Enrofloxacin (Baytril) | 259.3 ± 3.0 | 124.7 |
| Erythromycin (E-Mycin, Erythrocin) | 607.1 ± 5.0 | 297.0 |
| Ethambutol ((+)-S,S-Ethambutol) | 207.0 ± 3.0 | 98.8 |
| Etoposide | 378.5 ± 5.0 | 183.7 |
| Florfenicol (Aquafen, Nuflor) | 246.7 ± 3.0 | 118.4 |
| Flubendazol | 216.8 ± 3.0 | 103.6 |
| (Flumoxanal) | | |
| Fluconazole | 205.2 ± 7.0 | 97.9 |
| Flumequine | 179.4 ± 5.0 | 85.1 |
| 5-Fluorocytosine (Flucytosine) | 74.5 ± 7.0 | 33.1 |
| Flurbiprofen | 203.6 ± 3.0 | 97.1 |
| Formycin A (Adenosine, Formycin, NSC | 128.1 ± 7.0 | 59.7 |
| 102811) | | |
| Fumagillin | 383.2 ± 5.0 | 186.0 |
| Furazolidone | 135.5 ± 7.0 | 63.3 |
| Fusaric acid (5-Butylpicolinic acid) | 161.0 ± 3.0 | 75.9 |
| Fusidic acid | 443.4 ± 5.0 | 215.8 |
| G 418 | 336.3 ± 5.0 | 162.8 |
| Ganciclovir | 140.5 ± 7.0 | 65.8 |
| Gatifloxacin | 270.7 ± 3.0 | 130.3 |
| Gentamicin | 350.1 ± 5.0 | 169.6 |
| Gliotoxin | 186.5 ± 5.0 | 88.6 |
| gramicidin s | 919.5 ± 5.0 | 451.7 |
| Griseofulvin | 255.1 ± 5.0 | 122.6 |
| Herbimycin A | 479.9 ± 5.0 | 233.9 |
| Honokiol | 240.4 ± 3.0 | 115.3 |
| 8-Hydroxyquinoline | 115.2 ± 3.0 | 53.3 |
| 4-Hydroxytamoxifen ((Z)-Afimoxifene) | 354.6 ± 3.0 | 171.9 |
| Hygromycin B (WK2130000) | 315.1 ± 5.0 | 152.3 |
| Ikarugamycin | 390.4 ± 5.0 | 189.6 |
| Imipenem | 183.8 ± 7.0 | 87.2 |
| Ionomycin | 661.0 ± 3.0 | 323.7 |
| Irgasan | 194.3 ± 3.0 | 92.5 |
| Itraconazole | 502.0 ± 7.0 | 244.9 |
| Ivermectin Bla | 708.3 ± 5.0 | 347.1 |
| Josamycin | 684.5 ± 5.0 | 335.3 |
| K-252a | 278.7 ± 7.0 | 134.3 |
| Kanamycin | 297.5 ± 5.0 | 143.6 |
| Ketoconazole | 384.9 ± 7.0 | 186.9 |
| Kirromycin (mocimycin) | 622.6 ± 3.0 | 304.6 |
| Lactoferricin B (metallibure) | 184.7 ± 3.0 | 87.7 |
| Leptomycin A | 487.8 ± 3.0 | 237.9 |
| Leptomycin B | 504.3 ± 3.0 | 246.1 |
| Levamisol (Levamisole) | 154.1 ± 7.0 | 72.6 |
| Levofloxacin | 243.9 ± 5.0 | 117.1 |
| Lincomycin | 313.2 ± 5.0 | 151.4 |
| Lomefloxacin | 261.6 ± 3.0 | 125.8 |
| Lysobactin | 895.9 ± 7.0 | 440.1 |
| Magainin I | 1900.4 ± 3.0 | 937.7 |
| Mebendazole | 212.6 ± 3.0 | 101.6 |
| Meclocycline | 276.4 ± 5.0 | 133.2 |
| N-Methyl-1-deoxynojirimycin (1524564) | 127.0 ± 3.0 | 59.2 |
| Metronidazole | 117.8 ± 7.0 | 54. |
| Mevastatin | 343.8 ± 5.0 | 166.6 |
| Miconazole | 296.0 ± 7.0 | 142.9 |
| Minocycline | 294.5 ± 5.0 | 142.1 |
| Mithramycin A (Plicamycin) | 728.3 ± 5.0 | 357.0 |
| Mitomycin C (Mitomycin) | 213.6 ± 5.0 | 102.0 |
| Monensin | 552.7 ± 5.0 | 270.1 |
| Moxalactam (Latamoxef) | 292.5 ± 7.0 | 141.1 |
| Mupirocin | 423.0 ± 3.0 | 205.8 |
| Myxothiazol | 421.0 ± 3.0 | 204.8 |
| Nafcillin | 289.8 ± 5.0 | 139. |
| Naftifine | 265.4 ± 3.0 | 127.7 |
| Nalidixic acid | 174.4 ± 3.0 | 82.6 |
| Narasin | 650.1 ± 5.0 | 318.3 |
| Neocarzinostatin (Holoneocarzinostatin, NCS, | 434.9 ± 5.0 | 211.7 |
| NSC-69856, Zinostatin) | | |
| Neomycin | 380.3 ± 5.0 | 184.6 |
| Netilmicin | 358.8 ± 5.0 | 174.0 |
| Netropsin (Congocidin, Sinanomycin) | 282.7 ± 7.0 | 136.3 |
| Niclosamide | 202.4 ± 3.0 | 96.5 |
| Nigericin (Antibiotic K178, Antibiotic X464, | 607.9 ± 5.0 | 297.4 |
| Azalomycin M, Helexin C, Polyetherin A) | | |
| Nikkomycin | 300.9 ± 3.0 | 145.3 |
| Nitrofurantoin (Furadoxyl, Nitrofurantoine) | 131.0 ± 7.0 | 61.1 |
| Nonactin (Ammonium ionophore) | 707.9 ± 3.0 | 346.9 |
| Norfloxacin | 237.4 ± 3.0 | 113.8 |
| Novobiocin | 430.9 ± 5.0 | 209.7 |
| Nystatin (Fungicidin, Mycostatin) | 696.1 ± 5.0 | 341.1 |
| Ochratoxin A | 283.2 ± 3.0 | 136.5 |
| Ofloxacin | 243.9 ± 5.0 | 117.2 |
| Oligomycin A | 688.2 ± 5.0 | 337.2 |
| Oxacillin | 268.4 ± 5.0 | 129.2 |
| Oxolinic acid | 176.1 ± 3.0 | 83.4 |
| Oxytetracycline | 268.1 ± 5.0 | 129.1 |
| Paclitaxel | 610.5 ± 5.0 | 298.7 |
| Paromomycin | 374.1 ± 5.0 | 181.6 |
| Patulin | 101.3 ± 5.0 | 46.4 |
| PD 404, 182 | 150.9 ± 7.0 | 71.0 |
| Pefloxacin | 252.4 ± 3.0 | 121.3 |
| Penicillin G (Benzylpenicillin) | 235.1 ± 5.0 | 112.7 |
| Pentamidine | 281.4 ± 7.0 | 135.6 |
| Phenazine | 144.1 ± 3.0 | 67.6 |
| Phleomycin (UNII:BN3E7WJN9X) | 724.4 ± 7.0 | 355.1 |
| Phosphomycin (Fosfomycin) | 88.4 ± 5.0 | 40.0 |
| Pimaricin (Natamycin) | 477.4 ± 5.0 | 232.7 |
| Pipemidic acid | 219.6 ± 3.0 | 105.0 |
| Piperacillin | 340.5 ± 5.0 | 164.9 |
| Pirarubicin (THP) | 413.3 ± 5.0 | 200.9 |
| Polymyxin B | 941.8 ± 5.0 | 462.8 |
| Praziquantel | 254.2 ± 5.0 | 122.2 |
| PUROMYCIN | 311.7 ± 7.0 | 150.6 |
| Pyrazinecarboxamide (Pyrazinamide, | 87.7 ± 7.0 | 39.7 |
| Pyrazinoic acid amide) | | |
| Pyronaridine | 381.8 ± 3.0 | 185.4 |
| Pyrrolnitrin | 168.7 ± 3.0 | 79.8 |
| Quinine | 266.3 ± 5.0 | 128.2 |
| 8-Quinolinol (8-Hydroxyquinolin, Oxine) | 115.2 ± 3.0 | 53.3 |
| Radicicol | 267.3 ± 3.0 | 128.6 |
| Rapamycin (Sirolimus) | 773.4 ± 5.0 | 379.4 |
| Reveromycin A (MFCD00912537) | 542.2 ± 5.0 | 264.8 |
| Ribavirin | 117.0 ± 7.0 | 54.2 |
| Ribostamycin | 283.6 ± 5.0 | 136.7 |
| Rifabutin (Ansamycin, Ansatipine (Farmitalia), | 632.4 ± 7.0 | 309.5 |
| LM-427, Mycobutin (Farmitalia)) | | |
| Rifampicin (Rifampin, Rifamycin AMP) | 611.7 ± 7.0 | 299.3 |
| Rifapentine (DL 473) | 648.0 ± 7.0 | 317.3 |
| Rifaximin (Rifacol) | 575.3 ± 7.0 | 281.2 |
| Roxithromycin | 666.3 ± 7.0 | 326.3 |
| Salinomycin | 633.5 ± 5.0 | 310.1 |
| Sisomicin | 322.6 ± 5.0 | 156.1 |
| Sorbic acid | 109.4 ± 3.0 | 50.4 |
| Sordarin | 381.1 ± 5.0 | 185.0 |
| Sparfloxacin | 273.2 ± 3.0 | 131.6 |
| Spectinomycin | 231.6 ± 5.0 | 110.9 |
| Spergualin | 304.4 ± 7.0 | 147.0 |
| Spiramycin | 692.8 ± 5.0 | 339.4 |
| Staurosporine (Staurosporine) | 298.7 ± 7.0 | 144.2 |
| Streptomycin | 293.4 ± 7.0 | 141. |
| Streptonigrin (Bruneomycin, Nigrin) | 322.2 ± 5.0 | 155.8 |
| Streptozocin (Streptozotocin) | 142.5 ± 7.0 | 66.8 |
| Prothionamide | 158.5 ± 3.0 | 74.8 |
| Monensin | 552.7 ± 5.0 | 270.0 |

TABLE 2
Anticancer Drugs, Loading Time for Use In MVM2 Platform

| | Molar | Loading |
|---|---|---|
| Chemical, drug | volume (cm³) | time (s) |
| Abemaciclib (Verzenio) | 382.3 ± 7.0 | 185.6 |
| Abiraterone acetate (Zytiga) | 343.0 ± 5.0 | 166.1 |
| Acalabrutinib | 338.9 ± 7.0 | 164.1 |
| Afinitor (Everolimus) | 811.2 ± 5.0 | 398.1 |
| Aldara (Imiquimod) | 187.7 ± 7.0 | 89.2 |
| Alectinib | 374.7 ± 5.0 | 181.8 |
| Alemtuzumab | 165.7 ± 7.0 | 78.3 |
| Alimta (Pemetrexed Disodium) | 268.0 ± 7.0 | 129.0 |
| Aliqopa (Copanlisib Hydrochloride) | 317.6 ± 7.0 | 153.5 |
| Aloxi (Palonosetron Hydrochloride) | 238.6 ± 5.0 | 114.4 |
| Alunbrig (Brigatinib) | 443.6 ± 5.0 | 215.9 |
| Ameluz (Aminolevulinic Acid) | 106.4 ± 3.0 | 48.9 |
| Amifostine | 156.6 ± 3.0 | 73.8 |
| Anastrozole | 270.2 ± 7.0 | 130.1 |
| Apalutamide | 300.1 ± 5.0 | 144.9 |
| Aprepitant | 353.5 ± 7.0 | 171.4 |
| Arimidex (Anastrozole) | 270.2 ± 7.0 | 130.1 |
| Aromasin (Exemestane) | 260.5 ± 5.0 | 125.3 |
| Arranon (Nelarabine) | 149.8 ± 7.0 | 70.4 |
| Axitinib | 284.8 ± 5.0 | 137.3 |
| Azacitidine (Vidaza) | 117.0 ± 7.0 | 54.2 |
| Azedra | 148.6 ± 7.0 | 69.9 |
| Beleodaq (Belinostat) | 222.9 ± 3.0 | 106.7 |
| Bendamustine Hydrochloride | 271.7 ± 7.0 | 130.8 |
| Bexarotene (Targretin) | 334.2 ± 3.0 | 161.8 |
| Bicalutamide (cosodex) | 282.7 ± 5.0 | 136.3 |
| BiCNU (Carmustine) | 146.4 ± 7.0 | 68.8 |
| Binimetinib | 264.1 ± 7.0 | 127.1 |
| Bortezomib (Velcade) | 316.5 ± 3.0 | 153.0 |
| Bosutinib | 388.3 ± 5.0 | 188.6 |
| Braftovi (Encorafenib) | 371.6 ± 7.0 | 180.3 |
| Brigatinib | 443.6 ± 5.0 | 216.0 |
| Busulfan | 182.3 ± 3.0 | 86.5 |
| Cabazitaxel | 635.1 ± 5.0 | 310.9 |
| Calquence (Acalabrutinib) | 338.9 ± 7.0 | 164.1 |
| Campath (Alemtuzumab) | 165.7 ± 7.0 | 78.3 |
| Camptosar (Irinotecan Hydrochloride) | 416.8 ± 5.0 | 202.7 |
| Capecitabine (Xeloda) | 240.5 ± 7.0 | 115.4 |
| Carac (Fluorouracil-Topical, Tolak) | 84.5 ± 5.0 | 38.1 |
| Carfilzomib (Kyprolis) | 619.5 ± 3.0 | 303.1 |
| Carmustine | 146.4 ± 7.0 | 68.8 |
| Ceritinib (Zykadia) | 445.9 ± 3.0 | 217.1 |
| Cerubidine (Daunorubicin Hydrochloride) | 339.4 ± 5.0 | 164.4 |
| Chlorambucil | 243.6 ± 3.0 | 116. |
| Cladribine | 140.1 ± 7.0 | 65.6 |
| Clofarabine | 143.0 ± 7.0 | 67.1 |
| Cobimetinib | 311.3 ± 3.0 | 150.5 |
| Copanlisib Hydrochloride | 317.6 ± 7.0 | 153.6 |
| Copiktra (Duvelisib) | 282.8 ± 3.0 | 136.3 |
| Cosmegen (Dactinomycin) | 880.6 ± 7.0 | 432.5 |
| Crizotinib (Xalkori) | 305.2 ± 7.0 | 147.4 |
| Cyclophosphamide | 195.6 ± 5.0 | 93.1 |
| Cytarabine (Tarabine PFS) | 128.4 ± 7.0 | 59.8 |
| Dabrafenib (Tafinlar) | 359.8 ± 3.0 | 174.5 |
| Dacarbazine | 122.6 ± 7.0 | 56.9 |
| Dacogen (Decitabine) | 119.7 ± 7.0 | 55.5 |
| Dacomitinib (Vizimpro) | 349.4 ± 3.0 | 169.3 |
| Dasatinib | 346.4 ± 3.0 | 167.8 |
| Daunorubicin Hydrochloride | 339.4 ± 5.0 | 164.4 |
| Decitabine | 119.7 ± 7.0 | 55.5 |
| Degarelix | 1231.3 ± 3.0 | 606.2 |
| Dexamethasone | 296.2 ± 5.0 | 142.9 |
| Dexrazoxane Hydrochloride (Totect, | 201.2 ± 3.0 | 95.91218 |
| Zinecard) | | |
| Docetaxel (Taxotere) | 585.7 ± 5.0 | 286.4 |
| Doxorubicin | 336.6 ± 5.0 | 162.9 |
| Duvelisib | 282.8 ± 3.0 | 136.3 |
| Leuprolide Acetate) | 834.5 ± 7.0 | 409.6 |
| Ellence (Epirubicin Hydrochloride) | 336.6 ± 5.0 | 163.0 |
| Eltrombopag Olamine | 332.0 ± 7.0 | 160.7 |
| Emend (Aprepitant) | 353.5 ± 7.0 | 171.4 |
| Enasidenib Mesylate | 320.4 ± 3.0 | 155. |
| Enzalutamide (Xtandi) | 310.0 ± 5.0 | 149.8 |
| Epoetin Alfa | 665.6 ± 5.0 | 326. |
| Eribulin Mesylate | 563.2 ± 5.0 | 275.2 |
| Erivedge (Vismodegib) | 292.4 ± 3.0 | 141.1 |
| Erleada (Apalutamide) | 300.1 ± 5.0 | 144.9 |
| Erlotinib Hydrochloride (Tarceva) | 315.4 ± 5.0 | 152.5 |
| Ethyol (Amifostine) | 156.6 ± 3.0 | 73.8 |
| Etopophos (Etoposide Phosphate) | 406.7 ± 5.0 | 197.7 |
| Everolimus | 811.2 ± 5.0 | 398.1 |
| Evista (Raloxifene Hydrochloride) | 367.3 ± 3.0 | 178.2 |
| Evomela (Melphalan Hydrochloride) | 231.2 ± 3.0 | 110.8 |
| 5-FU (Fluorouracil Injection) | 84.5 ± 5.0 | 38.1 |
| Fareston (Toremifene) | 367.6 ± 3.0 | 178.3 |
| Farydak (Panobinostat) | 281.4 ± 3.0 | 135.6 |
| Faslodex (Fulvestrant) | 505.1 ± 3.0 | 246.5 |
| FEC | 75.0 ± 5.0 | 33.4 |
| Femara (Letrozole) | 234.5 ± 7.0 | 112.4 |
| Firmagon (Degarelix) | 1231.3 ± 3.0 | 606.2 |
| Fludarabine Phosphate | 152.5 ± 7.0 | 71.8 |
| Flutamide | 201.2 ± 3.0 | 95.9 |
| Folotyn (Pralatrexate) | 324.5 ± 3.0 | 156.9 |
| Fusilev (Leucovorin Calcium) | 280.7 ± 7.0 | 135.3 |
| Gefitinib | 337.7 ± 3.0 | 163.5 |
| Gemcitabine Hydrochloride | 142.3 ± 7.0 | 66.7 |
| Gilotrif (Afatinib Dimaleate) | 351.9 ± 3.0 | 170.6 |
| Gleevec (Imatinib Mesylate) | 393.0 ± 3.0 | 190.9 |
| Gliadel Wafer (Carmustine Implant) | 146.4 ± 7.0 | 68.8 |
| Goserelin Acetate (Zoladex) | 844.7 ± 7.0 | 414.7 |
| Granisetron | 234.8 ± 7.0 | 112.6 |
| Halaven (Eribulin Mesylate) | 563.2 ± 5.0 | 275.2 |
| Hemangeol (Propranolol Hydrochloride) | 237.1 ± 3.0 | 113.6 |
| Hycamtin (Topotecan Hydrochloride) | 281.3 ± 5.0 | 135.5 |
| Hydrea (Hydroxyurea) | 52.1 ± 3.0 | 22. |
| Ibrance (Palbociclib) | 340.7 ± 3.0 | 165.0 |
| Ibritumomab Tiuxetan (Zevalin) | 399.8 ± 7.0 | 194.2 |
| Ibrutinib | 327.4 ± 7.0 | 158.4 |
| Iclusig (Ponatinib Hydrochloride) | 412.2 ± 7.0 | 200.4 |
| Idarubicin Hydrochloride | 317.8 ± 5.0 | 153.6 |
| Idelalisib (Zydelig) | 282.2 ± 7.0 | 136.0 |
| Ifex (Ifosfamide) | 195.6 ± 5.0 | 93.1 |
| Imiquimod | 187.7 ± 7.0 | 89.2 |
| Ipilimumab (Yervoy) | 280.8 ± 3.0 | 135.3 |
| Istodax (Romidepsin) | 468.4 ± 3.0 | 228.2 |
| Ivosidenib (Tibsovo) | 383.5 ± 5.0 | 186.2 |
| Ixabepilone | 451.5 ± 3.0 | 219.9 |
| Ixazomib Citrate | 351.7 ± 5.0 | 170.4 |
| Ruxolitinib phosphate | 218.6 ± 7.0 | 104.5 |
| Kisqali (Ribociclib) | 635.1 ± 5.0 | 310. |
| Lanreotide Acetate | 781.0 ± 5.0 | 383. |
| Larotrectinib Sulfate (Vitrakvi) | 274.6 ± 7.0 | 132.2 |
| Lenalidomide | 177.4 ± 3.0 | 84.1 |
| Lenvatinib Mesylate | 290.5 ± 5.0 | 140.1 |
| Lomustine | 173.0 ± 7.0 | 81.9 |
| Lorbrena (Lorlatinib) | 285.0 ± 7.0 | 137. |
| Lupron (Leuprolide Acetate) | 834.5 ± 7.0 | 409.6 |
| Lynparza (Olaparib) | 301.7 ± 7.0 | 145.6 |
| Marqibo (Vincristine Sulfate Liposome) | 586.8 ± 5.0 | 286.9 |
| Matulane (Procarbazine Hydrochloride) | 213.6 ± 3.0 | 102.1 |
| Mechlorethamine Hydrochloride | 141.0 ± 3.0 | 66.1 |
| Megestrol Acetate | 333.3 ± 5.0 | 161.3 |
| Mekinist (Trametinib) | 353.0 ± 5.0 | 171.1 |
| Melphalan | 231.2 ± 3.0 | 110.7 |
| Mercaptopurine | 94.1 ± 3.0 | 42.8 |
| Methotrexate | 295.6 ± 3.0 | 142.6 |
| Midostaurin | 385.6 ± 7.0 | 187.2 |
| Mitomycin C | 213.6 ± 5.0 | 102.0 |
| Mitoxantrone Hydrochloride | 306.5 ± 3.0 | 148.0 |
| Mozobil (Plerixafor) | 522.2 ± 3.0 | 254.9 |
| Mustargen (Mechlorethamine | 141.0 ± 3.0 | 66. |
| Hydrochloride) | | |
| Myleran (Busulfan) | 182.3 ± 3.0 | 86.5 |
| Navelbine (Vinorelbine Tartrate) | 569.7 ± 5.0 | 278.4 |
| Nelarabine | 149.8 ± 7.0 | 70.4 |
| Neratinib Maleate | 416.7 ± 5.0 | 202.6 |
| Neulasta (Pegfilgrastim) | 252.4 ± 3.0 | 121.2 |
| Nexavar (Sorafenib Tosylate) | 319.4 ± 3.0 | 154.4 |
| Nilandron (Nilutamide) | 216.7 ± 3.0 | 103.5 |
| Nilotinib (Tasigna) | 388.6 ± 7.0 | 188.7 |
| Ninlaro (Ixazomib Citrate) | 351.7 ± 5.0 | 170.4 |
| Odomzo (Sonidegib) | 386.7 ± 3.0 | 187.8 |
| Omacetaxine Mepesuccinate | 408.4 ± 5.0 | 198.5 |
| Osimertinib (Tagrisso) | 418.4 ± 7.0 | 203.5 |
| Paclitaxel (Taxol) | 610.5 ± 5.0 | 298 |
| Loading time (s) | | |
| PAD | 299.1 ± 7.0 | 144. |
| Palbociclib | 340.7 ± 3.0 | 165. |
| Palonosetron Hydrochloride | 238.6 ± 5.0 | 114.4 |
| Panobinostat | 281.4 ± 3.0 | 135. |
| Pazopanib Hydrochloride (Votrient) | 310.3 ± 7.0 | 149.9 |
| Pegfilgrastim (Zarxio) | 252.4 ± 3.0 | 121.2 |
| Pomalidomide | 173.9 ± 3.0 | 82.3 |
| Prednisone | 273.6 ± 5.0 | 131.7 |
| Procarbazine Hydrochloride | 213.6 ± 3.0 | 102.0 |
| Promacta (Eltrombopag Olamine) | 332.0 ± 7.0 | 160.7 |
| Purinethol | 94.1 ± 3.0 | 42.8 |
| Raloxifene Hydrochloride | 367.3 ± 3.0 | 178.2 |
| Regorafenib | 323.6 ± 3.0 | 156.5 |
| Ribociclib | 311.4 ± 7.0 | 150.5 |
| Rheumatrex (Methotrexate, Trexall) | 295.6 ± 3.0 | 142.6 |
| Rolapitant Hydrochloride | 373.3 ± 5.0 | 181.1 |
| Romidepsin | 460.3 ± 3.0 | 224.2 |
| Rubidomycin (Daunorubicin | 339.4 ± 5.0 | 164.3 |
| Hydrochloride) | | |
| Rydapt (Midostaurin) | 385.6 ± 7.0 | 187.2 |
| Sancuso (Granisetron) | 234.8 ± 7.0 | 112.5 |
| Somatuline Depot (Lanreotide Acetate) | 781.0 ± 5.0 | 383.1 |
| Sonidegib | 386.7 ± 3.0 | 187.8 |
| Stivarga (Regorafenib) | 323.6 ± 3.0 | 156. |
| Tabloid (Thioguanine) | 96.4 ± 3.0 | 43.9 |
| Temodar (Temozolomide) | 98.3 ± 7.0 | 44.9 |
| Temsirolimus (Torisel) | 853.1 ± 5.0 | 418.8 |
| Thalidomide | 171.7 ± 3.0 | 81.3 |
| Thiotepa | 125.7 ± 5.0 | 58.5 |
| Toremifene | 367.6 ± 3.0 | 178.3 |
| Trabectedin (Yondelis) | 489.2 ± 5.0 | 238.5 |
| Treanda (Bendamustine Hydrochloride) | 271.7 ± 7.0 | 130.8 |
| Trifluridine and Tipiracil Hydrochloride | 179.8 ± 3.0 | 179.8 |
| Uridine Triacetate (Vistogard) | 257.5 ± 5.0 | 257.5 |
| Valrubicin | 469.8 ± 5.0 | 469.8 |
| Vandetanib | 337.9 ± 3.0 | 337.9 |
| Varubi (Rolapitant Hydrochloride) | 373.3 ± 5.0 | 373.3 |
| Vemurafenib (Zelboraf) | 332.3 ± 3.0 | 332.3 |
| Venclexta (Venetoclax) | 647.7 ± 3.0 | 647.7 |
| Vinblastine Sulfate | 590.0 ± 5.0 | 590.0 |
| Vismodegib | 292.4 ± 3.0 | 292.4 |
| Vorinostat (Zolinza) | 224.9 ± 3.0 | 224.9 |
| Xospata (Gilteritinib Fumarate) | 444.9 ± 3.0 | 444.9 |
| Zofran (Ondansetron Hydrochloride) | 230.1 ± 7.0 | 230.1 |
| Zoledronic Acid (Zometa) | 127.3 ± 7.0 | 127.3 |

TABLE 3
Antifungal Drugs and Other Molecules, Loading Times

| | Molar | Loading |
|---|---|---|
| Class | volume (cm³) | time (s) |
| D-fructose | 113.3 | 56.1 |
| Glucose | 113.9 | 56.4 |
| Galactose | 104.0 | 51.5 |
| Antimycin | 431.0 ± 5.0 | 213.5 |
| Bleomycin | 887.0 ± 5.0 | 439.4 |
| 5-Bromo-5-nitro-1,3-dioxane | 115.3 ± 5.0 | 57.1 |
| Cinnamycin | 1281.0 ± 7.0 | 634.6 |
| Fengycin (Plipastatin) | 1105.9 ± 5.0 | 547.9 |
| Filastatin | 269.8 ± 3.0 | 133.7 |
| Filipin | 563.7 ± 3.0 | 279.3 |
| Gentian Violet | 340.9 ± 5.0 | 168.9 |
| Sinefungin | 199.3 ± 7.0 | 98.7 |
| Kasugamycin | 192.2 ± 7.0 | 95.2 |
| Magnolol (2,2′-Bichavicol, 5,5′- | 240.4 ± 3.0 | 119.1 |
| Diallyl-2,2′-biphenyldiol) | | |
| Oligomycin (Oligomycin A) | 688.2 ± 5.0 | 340.9 |
| Surfactin | 998.6 ± 3.0 | 494.7 |
| Terconazole | 391.5 ± 7.0 | 193.9 |
| Thiabendazole (2-(4- | 143.0 ± 3.0 | 70.8 |
| Thiazolyl)benzimidazole) | | |
| Thiolutin | 146.5 ± 5.0 | 72.6 |
| Thymol (5-Methyl-2-isopropylphenol) | 150.2 | 74.4 |
| Tioconazole | 269.0 ± 7.0 | 133.3 |
| Tolnaftate | 251.1 ± 3.0 | 124.4 |
| Tubercidin | 139.5 ± 7.0 | 69.1 |
| Terbinafine | 289.1 | 143.2 |
| Ketoconazole | 389.4 | 192.9 |
| Fluconazole | 205.2 | 101.7 |
| Itraconazole | 502.7 | 249.1 |
| Voriconazole | 244.7 | 121.2 |
| Caspofungin | 803.3 | 397.9 |
| Flucytosine | 74.5 | 36.9 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A microfluidic circuit comprising:
an inlet port;
an outlet port;
a main channel fluidically connecting the inlet port and the outlet port; and
a series of dead-end microchambers of differing volumes, wherein each microchamber is individually fluidically connected to the main channel via a side channel.

2. The microfluidic circuit of claim 1, wherein the side channels comprise a passage having a lower volume capacity than that of the main channel.

3. The microfluidic circuit of claim 1 or claim 2, wherein the side channels are each identical in size, shape, and volume capacity.

4. The microfluidic circuit of any one of claims 1-3, wherein the side channel comprises a serpentine configuration.

5. The microfluidic circuit of any one of claims 1-4, wherein the side channel comprises an opening width of 40-100 μm, the side channel comprises a serpentine configuration with a switchback length at the shortest distance of 500-1500 μm, the microchambers comprise a diameter between 200-1500 μm, the inlet port comprises a diameter of 500-1500 μm, and the outlet port comprises a diameter of 500-1500 μm.

6. The microfluidic circuit of any one of claims 1-5, wherein the circuit comprises 2, 3, 4, 5, 6, 7, 8, 9, 10, 11, 12, 13, 14, 15, 16, 17, 18, 19, 20, 21, 22, 23, 24 or more than 24 microchambers.

7. The microfluidic circuit of any one of claims 1-6, wherein the microchambers comprise a circular shape.

8. The microfluidic circuit of any one of claim 7, wherein each microchamber increases in diameter as its position increases in distance from the inlet port.

9. The microfluidic circuit of any one of claims 1-8, wherein the main channel comprises a linear shape.

10. The microfluidic circuit of any one of claims 1-9, wherein the series of microchambers are arranged in size of graduated volumes from lowest to highest from the inlet port towards the outlet port.

11. The microfluidic circuit of any one of claims 1-10, wherein the series of microchambers are arranged in equally-sized pairs positioned on either side of the main channel.

12. The microfluidic circuit of any one of claims 1-11, wherein the inlet port and outlet port comprise a blocking element.

13. A microfluidic device comprising:
a support layer;
a substrate layer disposed on the support layer; and
one or more microfluidic circuits of any one of claims 1-11, wherein the one or more circuits are disposed within the substrate layer.

14. The microfluidic device of claim 13 comprising at least two microfluidic circuits, wherein the at least two of the microfluidic circuits are disposed within the substrate layer.

15. The microfluidic device of claim 14, further comprising:
a connecting channel connecting the at least two circuits.

16. The microfluidic device of claim 15, wherein the connecting channel is adjacent to the outlet ports.

17. The microfluidic device of any one of claims 14-16, wherein the support layer comprises glass.

18. The microfluidic device of any one of claims 14-17, wherein the substrate layer comprises polydimethylsiloxane (PDMS).

19. The microfluidic device of any one of claims 14-18 further comprising:
a top surface contiguous with the substrate layer.

20. The microfluidic device of any one of claims 14-19 further comprising:
a top surface contiguous with the substrate layer, wherein the top surface comprises polydimethylsiloxane (PDMS).

21. A method for performing an assay, said method comprising:
loading a first reagent solution into the inlet port of a microfluidic device of any one of claims 13-20;
loading a second reagent solution into the inlet port;
loading an isolating solution into the inlet port; and
detecting an interaction between the first reagent solution and the second reagent solution in one or more of the microchambers.

22. The method of claim 21, wherein the first reagent solution fills the microchambers.

23. The method of claim 21 or claim 22, wherein a portion of the second reagent solution diffuses into the microchambers.

24. The method of any one of claims 21-23, wherein a portion of the second reagent solution diffuses into the microchambers, thereby forming a concentration gradient of the second reagent solution within the microchambers from the inlet port to the outlet port.

25. The method of any one of claims 21-24, wherein the inlet and outlet ports of circuits that are not being loaded with the second reagent solution or the isolating solution are blocked.

26. The method of any one of claims 21-25, wherein the isolating solution prevents diffusion of the first reagent solution and second reagent solution from the microchambers.

27. The method of any one of claims 21-26, wherein the first reagent solution comprises a biological sample.

28. The method of claim 27, wherein the biological sample comprises a prokaryotic cell or prokaryotic cell component.

29. The method of claim 27, wherein the biological sample comprises a eukaryotic cell or a eukaryotic cell component.

30. The method of any one of claims 21-29, wherein the second reagent solution comprises an antimicrobial compound.

31. The method of any one of claims 21-30, wherein the isolating solution comprises a biocompatible oil.

32. The method of claim 29, wherein the biological sample comprises a cancer cell.

33. The method of claim 32, wherein the second reagent solution comprises an anti-cancer agent.
